(19) United States
(12) Patent Application Publication
(10) Pub. No.: US 2022/0289787 A1
(43) Pub. Date: Sep. 15, 2022

(54) DIMER COMBINATIONS AND USES THEREOF

(71) Applicant: Ripple Therapeutics Corporation, Toronto (CA)

(72) Inventors: Ian Charles PARRAG, Mississauga (CA); Matthew Alexander John STATHAM, Milton (CA); J. Paul SANTERRE, Toronto (CA); Wendy Alison NAIMARK, Toronto (CA); Adam Bruce DALEY, Toronto (CA)

(21) Appl. No.: 17/629,733

(22) PCT Filed: Jul. 24, 2020
(86) PCT No.: PCT/IB2020/000620

§ 371 Date: Jan. 24, 2022

Related U.S. Application Data
(60) Provisional application No. 62/878,477, filed on Jul. 25, 2019.

(51) Int. Cl. C07J 5/00; A61K 47/55

(52) U.S. Cl. CPC C07J 5/0053; A61K 47/55

(57) ABSTRACT

The present invention is directed towards articles comprising a steroid material comprising a steroid dimer and at least one therapeutic agent distributed throughout the steroid material and the use of such articles in sustained release delivery systems. Said article may be fibers, fiber meshes, woven fabrics, non-woven fabrics, films, pellets, cylinders, microparticles, nanoparticles, shaped articles or coatings on a substrate (such as an implant). Control of the release profile of the at least one therapeutic agent can be achieved via selection of the steroids within the steroid dimer, the linker conjugating the steroids within the dimer, the linkage groups between the steroids and the linker, the surface area of the article, the location of the at least one therapeutic agent within the article and the amount of the at least one therapeutic agent within the article.

### CROSS-REFERENCE

This application claims the benefit of U.S. Provisional Application No. 62/878,477, filed Jul. 25, 2019, which is hereby incorporated by reference in its entirety herein.

### BACKGROUND

The clinical importance of sustained drug release delivery systems to maintain therapeutic concentration of drugs for extended periods of time (e.g., days to weeks, to months or even years) has been well acknowledged for decades. Drug delivery systems with engineerable release kinetics are important and have application in multiple fields of medicine. In some embodiments, the ability to deliver different therapeutics and, in certain applications, deliver two or more active drugs as combination therapy is desirable. To develop successful sustained drug delivery systems, many technical difficulties must be overcome. In various instances, such difficulties include, by the way of non-limiting example, drug degradation during formulation process; lack of controlled release, including unwanted burst or incomplete release associated with diffusion or bulk erosion mechanisms of drug release; low encapsulation efficiency; formulation complexity; combinations of such difficulties; and/or other difficulties. The challenge is particularly difficult where a combination of drugs is delivered simultaneously, each in a predetermined release profile.

### SUMMARY

Provided in certain embodiments herein are multi-component systems, such as comprising a first component and a second component. In some embodiments, the first component comprises a drug dimer, such as a (e.g., steroid) dimer described herein. In some embodiments, the second component is a biologically active agent, such as a therapeutic agent described herein, for example, that when combined with the first component provides an additive or synergistic (e.g., biological) effect (e.g., in an individual). In some embodiments, the first component and the second component are (e.g., uniformly) mixed with each other (e.g., the second component is dispersed (e.g., uniformly) within the first component). In some embodiments, the first component and the second component are combined to form an article (e.g., an implantable article) or a pharmaceutical composition described herein. In some embodiments, provided herein is an (e.g., implant) article comprising a first component and a second component, such as described herein. In certain embodiments, provided herein is a composition comprising a first component and a second component, such as described herein. In some embodiments, the second component is combined (e.g., mixed) with the first component such as in an article or composition provided herein, such that the article or composition releases the first component (e.g., or at least a portion thereof, such as, for example, a monomer (e.g., in its free form) of a steroid dimer), the second component, or a combination thereof (e.g., into an environment into which it has been placed, such as a liquid medium, an individual (e.g., an implant location), or the like. In some embodiments, such release provides an additive or a synergistic effect (e.g., in an individual implanted with an article comprising the first component and the second component).

In certain embodiments, a first component described herein comprises any suitable compound provided herein, such as a compound of formula (A-1):

D1-L-D2 (A-1),
or a pharmaceutically acceptable salt thereof.

In certain embodiments, each of D1 and D2 is, independently, a radical formed from a steroid (e.g., also referred to herein as a steroid or a steroid radical). In certain embodiments, L is a linker joining D1 to D2. In specific embodiments, Lisa linker that covalently joins D1 to D2.

In some embodiments, the first component comprises any suitable amount of the compound (e.g., of formula (A-I)). In some embodiments, the first component comprises at least 50% (w/w) (e.g., at least 60% (w/w), at least 70% (w/w), at least 80% (w/w), at least 90% (w/w), at least 95% (w/w), at least 98% (w/w), at least 99% (w/w), or the like) of the compound (e.g., of formula (A-I)). In some embodiments, the first component is free or substantially free of a controlled release excipient, such as a controlled release (e.g., polymer) matrix.

In some embodiments, a second component is uniformly mixed with the first component (e.g., providing an article configured to release at least a portion of the first component or the second component at a controlled rate). In certain embodiments, (e.g., even without or with low concentrations of a controlled-release excipient) an article comprising (e.g., high concentrations, such as described herein) of the first component and the second component have good release profiles and/or kinetics. For example, in some instances, extended release of the article and/or its component parts (e.g., the therapeutic agent and/or D1 and D2 in their free (non-radical) forms) in tissue (e.g., of an individual), serum (e.g., of an individual or bovine serum (e.g., as a standard utilized to measure release kinetics and/or profile)), or a buffer (such as phosphate buffered saline, PBS). Such release profiles are determined at any suitable temperature, such as about 37° C. (e.g., in the body of an individual, or as a temperature to mimic the temperature of an individual). In some embodiments, extended release of the first component (e.g., or at least a portion thereof (e.g., D1 and/or D2 in their free form)) and/or the second component (e.g., the therapeutic agent) is achieved over a period of at least 1 month, at least 2 months, at least 3 months, or more (e.g., under the conditions described herein). In certain embodiments, release of the first component (e.g., or at least a portion thereof (e.g., D1 and/or D2 in their free form)) and/or the second component (e.g., the therapeutic agent) is zero order or near zero order.

In some embodiments, the first and second components collectively comprises any suitable amount of an article, implant, or composition provided herein. In some embodiments, the first and second components collectively comprises at least 50% (w/w) (e.g., at least 60% (w/w), at least 70% (w/w), at least 80% (w/w), at least 90% (w/w), at least 95% (w/w), at least 98% (w/w), at least 99% (w/w), or the like) of the article or composition. In some embodiments, the article or composition is free or substantially free of a controlled release excipient, such as a controlled release (e.g., polymer) matrix (e.g., that is not the first component)

Provided in certain instances herein is an article comprising a steroid material and at least one therapeutic agent, the steroid material comprising a compound of formula (A-I):

D1-L-D2 (A-I)

or a pharmaceutically acceptable salt thereof,
wherein:
(i) each of D1 and D2 is, independently, a steroid radical; and Lisa linker covalently linking D1 to D2; and
(ii) the at least one therapeutic agent is distributed (e.g., uniformly) throughout the steroid material.

In some embodiments, the at least one therapeutic agent is uniformly distributed or mixed with or within the steroid material (e.g., in such a way that the release of the steroid material (e.g., a free form of D1 and/or D2) and/or therapeutic agent from the article is zero-order or near zero-order). In some embodiments, the article comprises a homogeneous mixture of the steroid material and the at least one therapeutic agent. In some embodiments, the article comprises a heterogeneous mixture of the steroid material and the at least one therapeutic agent. In some embodiments, the homogenous mixture and/or the heterogenous mixture of the steroid material and the at least one therapeutic agent provides an article configured to release the steroid material (e.g., the components of the steroid material (e.g., D1 and/or D2 in their free (non-radical) form)) and/or the at least one therapeutic agent at a controlled (e.g., an extended or sustained) rate.

In some embodiments, the controlled rate is near zero order or zero order. In some embodiments, the controlled rate is zero order. In some embodiments, the controlled rate is near zero order. In some embodiments, the controlled rate is near zero-order or zero-order, and the formulation is a homogeneous mixture of the steroid material and the at least one therapeutic agent. In some embodiments, the controlled rate is near zero-order or zero-order, and the formulation is a heterogeneous mixture of the steroid material and the at least one therapeutic agent.

In some embodiments, the homogeneous mixture has a release rate that is closer to zero order than the heterogeneous mixture. In some embodiments, the homogeneous mixture has a release rate that is closer to the release rate of the steroid material than the heterogeneous mixture. In some embodiments, the homogeneous mixture has a release rate that is (e.g., substantially) the same as the release rate of the steroid material. In some embodiments, the heterogeneous mixture has a release rate that is (e.g., substantially) the same as the release rate of the steroid material. In some embodiments, the heterogeneous mixture provides a formulation that produces a burst release of the at least one therapeutic agent. In some embodiments, the heterogeneous mixture provides a formulation that produces a burst release of the at least one therapeutic agent and a near-zero order or a zero-order release rate thereafter. In some embodiments, the release rate of the homogeneous mixture of the heterogeneous mixture is tuned to produce a release rate provided herein (e.g., which release rate is configured to provide a release rate for an indication, disease, or disorder described herein).

In some embodiments, the article comprises one or more portions of the steroid material. In some embodiments, the article comprises a first portion of the steroid material and a second portion of the steroid material. In some embodiments, the first portion of the steroid material comprises the at least one therapeutic agent distributed throughout the steroid material. In some embodiments, the second portion of the steroid material comprises the at least one therapeutic agent distributed throughout the steroid material. In some embodiments, the at least one therapeutic agent is distributed homogeneously throughout the steroid material. In some embodiments, the at least one therapeutic agent is distributed heterogeneously throughout the steroid material. In some embodiments, the second portion of steroid material is free of the at least one therapeutic agent. In some embodiments, the first portion of the steroid material comprises the at least one therapeutic agent distributed throughout the steroid material, and the second portion of steroid material is free of the at least one therapeutic agent.

In some embodiments, the first portion of the steroid material is a layer of the article. In some embodiments, the first portion of the steroid material is an inner layer of the article (e.g., distal from one or more surface of the article). In some embodiments, the first portion of the steroid material is an outer layer of the article. In some embodiments, the first portion of the steroid material is an outer layer of the article that coats the second portion of the steroid material.

In some embodiments, the second portion of the steroid material is a layer of the article. In some embodiments, the second portion of the steroid material is an inner layer of the article. In some embodiments, the second portion of the steroid material is an outer layer of the article that coats the first portion of the steroid material.

In some embodiments, provided herein is an article comprising at least two layers (e.g., three layers, four layers, five layers, or more). In some embodiments, the at least two layers comprises the first portion and the second portion. In some embodiments, the article comprises one or more layer of the first portion, the second portion, or any combination thereof. In some embodiments, the one or more layers of the article are continuously repeated throughout the article. In some embodiments, at least one of the one or more layers of the article are configured to produce an immediate release of the steroid material (e.g., D1 and/or D2 in their free (non-radical) form)) and/or the at least one therapeutic agent (e.g., in an individual). In some embodiments, at least one of the one or more layers of the article are configured to produce a controlled (e.g., sustained or extended) release of the steroid material (e.g., D1 and/or D2 in their free (non-radical) form)) and/or the at least one therapeutic agent (e.g., in an individual).

In some embodiments, the article comprises less than 5 wt. %, less than 2 wt. %, or less than 1 wt. % of a controlled release excipient. In some embodiments, the steroid material comprises less than 5 wt. %, less than 2 wt. %, or less than 1 wt. % of a controlled release excipient. In some embodiments, the steroid material (e.g., the second body or the coating) is free of a controlled release excipient. In some embodiments, the at least one therapeutic agent and the steroid material together produce an article having a particular release profile (e.g., immediate release or controlled release).

In some embodiments, the steroid material and at least one therapeutic agent are released from the article at (e.g., substantially) the same rate. In some embodiments, the ratio of the steroid material and at least one therapeutic agent affects the steroid material and at least one therapeutic agent being released from the article at (e.g., substantially) the same rate. In some embodiments, the ratio of the steroid material and at least one therapeutic agent does not affect the steroid material and at least one therapeutic agent being released from the article at (e.g., substantially) the same rate.

In some embodiments, the steroid material and at least one therapeutic agent are released from the article at different rates. In some embodiments, the ratio of the steroid material and at least one therapeutic agent affects the steroid material and at least one therapeutic agent being released from the article at (e.g., substantially) different rates. In some embodiments, the ratio of the steroid material and at least one therapeutic agent does not affect the steroid material and at least one therapeutic agent being released from the article at different rates.

In some embodiments, the article comprises from 45% to 99% (w/w) (e.g., at least 45% w/w, at least 55% w/w, at least 65% w/w, at least 75% w/w, at least 85% w/w, at least 95% w/w, at least 99% w/w, or more) of the compound of formula (A-I). In some embodiments, the article comprises less than or equal to 45% w/w (e.g., at most 45% w/w, at most 35% w/w, at most 25% w/w, at most 15% w/w, at most 5% w/w, or less) of the compound of formula (A-I). In some embodiments, the article comprises more than or equal to 90% w/w of the compound of formula (A-I).

In some embodiments, the article comprises from 1% to 55% (w/w) (e.g., at most 55% w/w, at least 45% w/w, at least 35% w/w, at least 25% w/w, at least 15% w/w, at least 5% w/w, at least 1% w/w, or less) of the at least one therapeutic agent. In some embodiments, the article comprises more than or equal to 55% w/w (e.g., at least 55% w/w, at least 65% w/w, at least 75% w/w, at least 85% w/w, at least 95% w/w, or more) of the at least one therapeutic agent. In some embodiments, the article comprises less than or equal to 10% w/w of the at least one therapeutic agent.

In some embodiments, the steroid material (or compound of a formula provided herein) and a (one or more) therapeutic agent provided herein collectively comprises any suitable amount of an article, implant, or composition provided herein. In some embodiments, the steroid material (or compound of a formula provided herein) and a (one or more) therapeutic agent collectively comprises at least 50% (w/w) (e.g., at least 60% (w/w), at least 70% (w/w), at least 80% (w/w), at least 90% (w/w), at least 95% (w/w), at least 98% (w/w), at least 99% (w/w), or the like) of the article or composition. In some embodiments, the article or composition is free or substantially free of a controlled release excipient, such as a controlled release (e.g., polymer) matrix (e.g., that is not the steroid material).

In some embodiments, the article has a (w/w) ratio of the compound of formula (A-I) to the at least one therapeutic agent from about 1:20 to about 1:1 (e.g., from about 1:20, from about 1:15, from about 1:10, from about 1:5, from about 1:2, or from about 1:1). In some embodiments, the article has a (w/w) ratio of the compound of formula (A-I) to the at least one therapeutic agent from 1:10 to 1:2. In some embodiments, the article has a (w/w) ratio of the compound of formula (A-I) to the at least one therapeutic agent of about 1:10. In some embodiments, the article comprises about 10% of the steroid material and about 90% of the at least one therapeutic agent. In some embodiments, the article has a (w/w) ratio of the compound of formula (A-I) to the at least one therapeutic agent of about 1:2. In some embodiments, the article comprises about 33% of the steroid material and about 66% of the at least one therapeutic agent.

In some embodiments, the article has a (w/w) ratio of the compound of formula (A-I) to the at least one therapeutic agent from about 20:1 to about 1:1 (e.g., from about 20:1, from about 15:1, from about 10:1, from about 5:1, from about 2:1, or from about 1:1). In some embodiments, the article has a (w/w) ratio of the compound of formula (A-I) to the at least one therapeutic agent from 10:1 to 2:1. In some embodiments, the article has a (w/w) ratio of the compound of formula (A-I) to the at least one therapeutic agent of about 10:1. In some embodiments, the article comprises about 90% of the steroid material and about 10% of the at least one therapeutic agent. In some embodiments, the article has a (w/w) ratio of the compound of formula (A-I) to the at least one therapeutic agent of about 2:1. In some embodiments, the article comprises about 66% of the steroid material and about 33% of the at least one therapeutic agent.

In some embodiments, the at least one therapeutic agent is released from the article with at least a portion of the steroid material (e.g., D1 and/or D2 in their free (non-radical) form)). In some embodiments, the at least one therapeutic agent is released from the article with D1 and/or D2 in their free form. In some embodiments, D1 and D2 are released from the article in their free form (e.g., at 37° C. in 100% bovine serum or at 37° C. in PBS) at a rate such that t₁₀ is greater than or equal to 1/10 of t₅₀. In some embodiments, the at least one therapeutic agent, D1, and D2 are released from the article in their free form (e.g., at 37° C. in 100% bovine serum or at 37° C. in PBS) at a rate such that t₁₀ is greater than or equal to 1/10 of t₅₀. In some embodiments, D1 (e.g., or a free form thereof), D2 (or a free form thereof), the at least one therapeutic agent, or any combination thereof is released from the article through surface erosion.

In some embodiments, less than or equal to 20% (w/w) (e.g., less than 20% w/w, less than 15% w/w, less than 10% w/w, or less than 5% w/w) of the at least one therapeutic agent (e.g., as a percentage of the total article) is released from the article at 37° C. in PBS over a period 2 days or more (e.g., more than 2 days, more than 5 days, or more than 10 days). In some embodiments, less than or equal to 20% (w/w) (e.g., less than 20% w/w, less than 15% w/w, less than 10% w/w, or less than 5% w/w) of the at least one therapeutic agent, D1, and/or D2 (e.g., as a percentage of the total article) is released from the article at 37° C. in PBS over a period 2 days or more (e.g., more than 2 days, more than 5 days, or more than 10 days).

In some embodiments, less than or equal to 20% (w/w) (e.g., less than 20% w/w, less than 15% w/w, less than 10% w/w, or less than 5% w/w) of D1 and/or D2 (e.g., as a percentage of the total article) are released from the article in their free form at 37° C. in PBS over a period 5 days or more (e.g., more than 5 days, more than 7 days, or more than 10 days). In some embodiments, less than or equal to 20% (w/w) (e.g., less than 20% w/w, less than 15% w/w, less than 10% w/w, or less than 5% w/w) of the at least one therapeutic agent, D1, and/or D2 (e.g., as a percentage of the total article) are released from the article in their free form at 37° C. in PBS over a period 5 days or more (e.g., more than 5 days, more than 7 days, or more than 10 days).

In some embodiments, more than or equal to 50% (w/w) (e.g., more than 50% w/w, more than 60% w/w, or more than 75% w/w) of the at least one therapeutic agent (e.g., as a percentage of the total article) is released from the article at 37° C. in PBS over a period 1 day or more (e.g., more than 1 day, more than 2 days, or more than 5 days). In some embodiments, more than or equal to 50% (w/w) (e.g., more than 50% w/w, more than 60% w/w, or more than 75% w/w) of the at least one therapeutic agent, D1, and/or D2 (e.g., as a percentage of the total article) is released from the article at 37° C. in PBS over a period 1 day or more (e.g., more than 1 day, more than 2 days, or more than 5 days).

In some embodiments, D1 and D2 are each a steroid provided herein, or pharmaceutically acceptable salts thereof, in their free form. In some embodiments, D1 and D2 are each an anti-inflammatory steroid, or pharmaceutically acceptable salts thereof, in their free form. In some embodiments, D1 and D2 are each an anti-inflammatory steroid provided herein, or pharmaceutically acceptable salts thereof, in their free form. In some embodiments, D1 and D2 are each a corticosteroid (e.g., as provided herein), or pharmaceutically acceptable salts thereof, in their free form. In some embodiments, D1 and D2 are each a glucocorticoid (e.g., as provided herein), or pharmaceutically acceptable salts thereof, in their free form. In some embodiments, D1 and D2 are each independently selected from dexamethasone, triamcinolone, triamcinolone acetonide, prednisolone, hydrocortisone, betamethasone, and prednisone, or pharmaceutically acceptable salts thereof, in their free form. In some embodiments, D1 and D2 are each dexamethasone, or pharmaceutically acceptable salts thereof, in their free form. In some embodiments, D1 and D2 are each intraocular pressure (IOP) lowering steroids, or pharmaceutically acceptable salts thereof, in their free form. In some embodiments, D1 and D2 are each intraocular pressure (IOP) lowering steroids provided herein, or pharmaceutically acceptable salts thereof, in their free form. In some embodiments, D1 and D2 are each anecortave, or pharmaceutically acceptable salts thereof, in their free form.

In some embodiments, the at least one therapeutic agent is a second agent described herein. In some embodiments, the at least one therapeutic agent is an analgesic, an opioid (e.g., an opioid agonist or an opioid antagonist), an antimicrobial, an anti-proliferative, a kinase inhibitor, a steroid, a non-steroidal anti-inflammatory drug (NSAID), an immunomodulatory agent, a prostaglandin, or a combination of more than one free drug. In some embodiments, the at least one therapeutic agent is a therapeutic agent described herein, or a pharmaceutically acceptable salt thereof. In some embodiments, the at least one therapeutic agent comprises a combination of therapeutic agents (e.g., which are specific to treating a particular disease, disorder, or condition described herein). In some embodiments, the at least one therapeutic agent comprises a first therapeutic agent and a second therapeutic agent. In some embodiments, the first therapeutic agent and the second therapeutic agent are of a different chemical class (e.g., an analgesic and an NSAID). In some embodiments, the first therapeutic agent and the second therapeutic agent are of the same chemical class (e.g., both are an NSAID).

In certain embodiments, provided herein is a method for treating or preventing a condition, a side-effect, or a complication of a (e.g., surgical) procedure performed on an individual, the method comprising implanting an article provided herein into the individual.

In certain embodiments, provided herein is a method for treating a disease or disorder in an individual, the method comprising implanting an article provided herein into the individual.

In some embodiments, the components of the article (e.g., the at least one therapeutic agent and/or D1 and/or D2 in their free (non-radical) form) provided herein is released into the individual for the duration of a treatment regimen for an individual. In some embodiments, the components of the article (e.g., the at least one therapeutic agent and/or D1 and/or D2 in their free (non-radical) form) are continuously released at a zero-order or near-zero order rate into the individual (e.g., for the duration of a treatment regimen of an individual). In some embodiments, the components of the article (e.g., the at least one therapeutic agent and/or D1 and/or D2 in their free (non-radical) form) provided herein are released into the individual for at least 1 day (e.g., more than 2 days, more than 5 days, more than 14 days, or more than 30 days). In some embodiments, the components of the article (e.g., the at least one therapeutic agent and/or D1 and/or D2 in their free (non-radical) form) provided herein are released into the individual for a period of more than or equal to 2 days (e.g., more than 2 days, more than 5 days, more than 14 days, or more than 30 days).

In some embodiments, the procedure is radiation (e.g., radiation therapy). In some embodiments, the procedure is a surgical procedure. In some embodiments, the surgical procedure is an ophthalmic surgical procedure. In some embodiments, the surgical procedure is an emergency surgical procedure. In some embodiments, the surgical procedure is a standard surgical procedure.

In some embodiments, the condition, the side-effect, or the complication of the (e.g., surgical) procedure is inflammation, cellular proliferation, an infection (e.g., a bacterial infection), clotting, bleeding, hypertension, hypotension, an immune response (e.g., an auto-immune response, anaphylaxis, or a rash), a fibrotic response (e.g., scarring), edema, or increased (e.g., intraocular) pressure.

In some embodiments, the disease or disorder is a cancer, a fibrotic disease or disorder, an infection (e.g., a bacterial infection), a cardiac disease or disorder, an angiogenic disease or disorder, or an ocular disease or disorder. In some embodiments, the disease or disorder is a chronic condition. In some embodiments, the disease or disorder is described herein.

In some embodiments, the second agent is an ocular therapeutic agent (e.g., a therapeutic agent that has activity for treating or reducing the symptoms of a disease or disorder of the eye, such as glaucoma). In some embodiments, the second agent is a glaucoma therapeutic agent (e.g., a therapeutic agent that has activity for treating or reducing the symptoms of glaucoma). In some embodiments, the at least one therapeutic agent is selected from the group consisting of a prostaglandin, a beta-blocker, a rho kinase inhibitor, an alpha-adrenergic agonist, a carbonic anhydrase inhibitor, and a cholinergic agent. In some embodiments the therapeutic agent is an anti-inflammatory agent and/or an intraocular pressure lowering agent.

In other embodiments, the second agent is an anti-VEGF kinase inhibitor for inhibiting unwanted vascularization associated with macular degeneration.

In some embodiments, the second agent is a therapeutic agent for treating a condition, a side-effect, ora complication of a (e.g., surgical) procedure performed on an individual. In some embodiments, the procedure is a surgical procedure. In some embodiments, the surgical procedure is an ophthalmic surgical procedure.

In some embodiments, the condition, the side-effect, or the complication of the (e.g., surgical) procedure is inflammation, an infection (e.g., a bacterial infection), or increased (e.g., intraocular) pressure.

In some embodiments, the second agent is a non-steroidal anti-inflammatory drug (NSAID), an antibiotic, an antimicrobial, a steroid, or the like.

In certain embodiments, provided herein is an article including an admixture of: (i) a compound of formula (A-I):

D1-L-D2 (A-I),

or a pharmaceutically acceptable salt thereof, wherein each of D1 and D2 is, independently, a radical formed from a steroid; and Lisa linker covalently linking D1 to D2; and (ii) a second agent, wherein the second agent is a therapeutic drug. In some embodiments the article is free of controlled release polymer. The second agent can be distributed homogenously within the admixture or distributed heterogeneously within the admixture. In some embodiments, the second agent is a combination of therapeutic drugs.

In certain instances, provided herein is an article including: (i) from 45% to 99% (w/w) (e.g., 50±5%, 60±5%, 70±5%, 80±5%, or 90±5% (w/w)) of a compound of formula (A-I):

D1-L-D2 (A-I),

or a pharmaceutically acceptable salt thereof, wherein each of D1 and D2 is, independently, a radical formed from a steroid; and L is a linker covalently linking D1 to D2; and (ii) from 1% to 55% (w/w) (e.g., 3±2%, 5±3%, 10±5%, 20±5%, 30±5%, 40±5%, or 50±5% (w/w)) of a second agent, wherein the second agent is a therapeutic drug or combination of drugs. In some embodiments the article is free of controlled release polymer. In some embodiments, the second agent is a combination of therapeutic drugs.

In some embodiments, L has a molecular range of 80 to 800 Da. The compound of formula A-I can be any compound of formula A-I described herein. In certain embodiments, the compound of formula A-I controls the release of the therapeutic drug. In some embodiments, the article is a coating on the surface of a device. In some embodiments, at least 70%, 80%, or 90% (w/w) of the article is a compound of formula (A-I). In certain embodiments less than 30%, 20%, 10%, or 5% (w/w) of the article is the second agent.

In some embodiments, the compound of formula (A-I) is formulated for sustained release and the second agent (e.g., a single therapeutic agent or a combination thereof) is formulated for sustained release. The article can include a composition including the second agent distributed (e.g., homogenously) within the compound of formula (A-I). In other embodiments, the article includes a first portion including the second agent distributed within the compound of formula (A-I) and a second portion (e.g., including the compound of formula (A-I)) free of the second agent. The first portion can be an outer layer of the article sitting atop the second portion. Alternatively, the second portion can be an outer layer of the article sitting atop the first portion. In some instances, (i) D1 and D2 are released from the article through surface erosion (e.g., and the surface erosion releases less than 20% of D1 or D2, as a percentage of the total drug, D1 or D2, present in the article in prodrug form, at 37° C. in PBS over 5 days); and (ii) the article releases less than 20% (w/w) of the second agent present in the article (e.g., at 37° C. in PBS over 2 days). In some embodiments, the article releases less than 20% (w/w) of the second agent present in the article at 37° C. in PBS over 3 days, 4 days, or 5 days.

In some embodiments, the compound of formula (A-I) is formulated for sustained release and the second agent is formulated for immediate release. In certain instances, (i) D1 and D2 are released from the article through surface erosion and the surface erosion releases less than 5% of D1 or D2, as a percentage of the total drug, D1 or D2, present in the article in prodrug form, at 37° C. in PBS over 5 days; and (ii) the article releases greater than 50% or 75% (w/w) of the second agent present in the article at 37° C. in PBS over 1 day.

In certain embodiments, the compound of formula (A-I) is formulated for sustained release and the second agent is formulated for delayed release. In certain instances, (i) D1 and D2 are released from the article through surface erosion and the surface erosion releases less than 5% of D1 or D2, as a percentage of the total drug, D1 or D2, present in the article in prodrug form, at 37° C. in PBS over 5 days; and (ii) the article releases less than 10% (w/w) of the second agent present in the article at 37° C. in PBS prior to the release of at least 50% (w/w) of the compound of formula (A-I). The article can be formed by coating a substrate including the second agent with the compound of formula (A-I).

In certain embodiments, the (w/w) ratio of the compound of formula (A-I) to the second agent is from 20:1 to 1:1 (e.g., from 20:1 to 15:1, 15:1 to 4:1, 4:1 to 2:1, or 2:1 to 1:1).

In some embodiments, provided herein is an article including an admixture of: (i) from 5% to 45% (w/w) (e.g., 7.5±2.5%, 10±2.5%, 12.5±2.5%, 15±2.5%, 20±5%, 25±5%, 30±5%, or 40±5% (w/w)) of a compound of formula (A-I):

D1-L-D2 (A-I),

or a pharmaceutically acceptable salt thereof, wherein each of D1 and D2 is, independently, a radical formed from a steroid; and L is a linker covalently linking D1 to D2; and (ii) from 55% to 95% (w/w) (e.g., 60±5%, 65±5%, 70±5%, 75±5%, 80±5%, 85±5%, or 90±5% (w/w)) of a second agent, wherein the second agent is a therapeutic drug. In some embodiments, the article is free of controlled release polymer. In some embodiments, the second agent is distributed heterogeneously within the admixture. The article can be an admixture of the second agent and the compound of formula (A-I), where the compound of formula (A-I) is present as a binder. In other embodiments, the compound of formula (A-I) controls the release of the second agent from the article. In one embodiment, the (w/w) ratio of the compound of formula (A-I) to the second agent is from 1:2 to 1:20 (e.g., from 1:2 to 1:4, 1:4 to 1:20, or 1:8 to 1:20). In some embodiments, the second agent is a mixture of 2 or more therapeutic agents.

In some embodiments, the second agent has a molecular weight of from 50 to 10,000 Da (e.g., 100±50, 250±100, 500±250, 1000±200, 3000±1000, or 6000±4000 Da).

In some embodiments, L is covalently linked to D1 and to D2 via one or more ester, carbonate, carbonate ester, or anhydride linkages. In some embodiments, L is covalently linked to D1 and to D2 via one or more carbonate linkages.

In certain embodiments, L includes the radical —C(O)—(R^{A})—C(O)— or —O—(R^{A})—O—; R^{A} is a radical of a polyol and includes at least one free hydroxyl group or R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, —(CH₂CH₂O)_{q}CH₂CH₂—, —(CH₂CH₂CH₂CH₂O)ᵣCH₂CH₂CH₂CH₂—, or —(CH₂CH(CH₃)O)ₛCH₂CH(CH₃)—; and q, r, and s are integers from 1 to 10.

In certain embodiments, the compound of formula (A-I) is further described by one of formulas (II)-(LXXVIII). In other embodiments, the compound can be further described by the formula (A-II):

D1-O-L-O-D2 (A-II),

wherein each of D1-O and D2-O is, independently, described by any one of formulas (I-a) to (I-zzz). For example, upon hydrolysis D1 and D2 can form benign steroids selected from cholesterol, 11-deoxycortisol, 11-deoxycorticosterone, pregnenolone, cholic acid, chenodeoxycholic acid, ursodeoxycholic acid, and obeticholic acid. In some embodiments, upon hydrolysis D1 and D2 form corticosteroids selected from alclometasone, beclomethasone, betamethasone, betamethasone valerate, budesonide, chloroprednisone, cloprednol, corticosterone, cortisone, desonide, desoximerasone, dexamethasone, diflorasone, diflucortolone, enoxolone, flucloronide, flumethasone, flunisolide, fluocinolone acetonide, fluocortolone, fluprednisolone, flurandrenolide, halometasone, hydrocortisone, hydrocortisone butyrate, meprednisone, methylprednicolone, paramethasone, prednisolone, prednisone, prednival, prednylidene, triamcinolone, and triamcinolone acetonide. In some embodiments, the compound of formula (A-I) is further described by the formula (B-I):

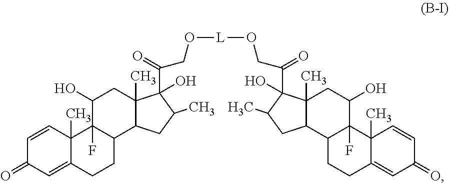

or a pharmaceutically acceptable salt thereof, wherein L is —C(O)O—(R^{A})—OC(O)—, —C(O)—OC(O)—(R^{A})—C(O)O—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; and n, m, and p are integers from 1 to 10.

In some embodiments, the article further includes from 0.1% to 10% (w/w) of one or more additives, wherein the one or more additives are selected from plasticizers, antioxidants, binders, lubricants, dyes, and mixtures thereof.

In some embodiments, the article provided herein is a fiber, fiber mesh, woven fabric, non-woven fabric, film, pellet, cylinder, microparticle, nanoparticle, a shaped article, or a coating on a substrate (e.g., a coating on an implantable device). In some embodiments, the article is a microparticle having a diameter of from about 1 to 100 μm. In other embodiments, the article is in the form of a cylinder, tube, fiber, or fiber mesh. In some embodiments, the article of the invention is a surface coating that is a patterned coating on the surface of the article selected from a checkerboard pattern, dot pattern, or striped pattern. These can be prepared, for example, by masking a portion or side of the surface being coated (e.g., the surface of a medical device) to produce a single-sided or patterned coating (e.g., a checkerboard pattern, dot pattern, or striped pattern). In some embodiments, the article includes two or more layers, wherein each of the two or more layers comprises a predetermined (w/w) percentage of the compound of formula (A-I) and the second agent.

In certain embodiments, the second agent is an analgesic, antimicrobial, anti-proliferative, kinase inhibitor, steroid, NSAID, statin, antihypertensive, vasodilator, antioxidant, neuroprotective agent, immunomodulatory agent, or prostaglandin or combination thereof. The second agent can be any second agent described herein. Any second agent described herein, or its pharmaceutically acceptable salt form, can be used in the articles of the invention. In some embodiments, the second agent is a steroid and the compound of formula (A-I) is formed from the second agent (e.g., the second agent is dexamethasone and the compound of formula (A-I) is a dimer of dexamethasone). In some embodiments, the second agent does not include a steroid. In other embodiments the second agent includes more than one free drug.

In some embodiments, an article provided herein is formed by a process including the steps of: (a) heating the compound of formula (A-I) and the second agent to form a melt; and (b) cooling the melt to form the article, wherein the compound of formula (A-I) and the second agent are formulated for sustained release.

In some embodiments, an article provided herein is formed by a process including the steps of: (a) heating the compound of formula (A-I) to form a melt; (b) mixing the melt with particles of the second agent; and (c) cooling the melt to form an admixture, wherein the second agent is distributed heterogeneously within the admixture and the compound of formula (A-I) and the second agent are formulated for sustained release. In other embodiments, the articles of the invention are formed by a process including the steps of: (a) mixing a solid powder of the compound of formula (A-I) with a solid powder of the second agent to form a mixture; (b) heating the mixture to a temperature above the melting point of the compound of formula (A-I) and below the melting point of the second agent to form a semi-melt; and (c) cooling the semi-melt to form an admixture, wherein the second agent is distributed heterogeneously within the admixture and the compound of formula (A-I) and the second agent are formulated for sustained release.

In some embodiments, an article provided herein is formed by a process including the steps of: (a) dissolving the compound of formula (A-I) and the second agent in a solvent to form a solution; and (b) evaporating the solvent to form the article, wherein the compound of formula (A-I) and the second agent are formulated for sustained release.

In some embodiments, an article provided herein is formed by a process including the steps of: (a) heating the compound of formula (A-I) to form a homogenous melt; (b) cooling the melt to form a core; and (c) coating the core with the second agent to form the article, wherein the compound of formula (A-I) is formulated for sustained release and the second agent is formulated for immediate release. Optionally, the coating includes the compound of formula (A-I) or a binding agent.

In one embodiment, an article provided herein is formed by a process including the steps of: (a) dissolving the compound of formula (A-I) in a solvent to form a solution; (b) evaporating the solvent to form a core; and (c) coating the core with the second agent to form the article, wherein the compound of formula (A-I) is formulated for sustained release and the second agent is formulated for immediate release. Optionally, the coating includes the compound of formula (A-I) or a binding agent.

In certain embodiments, an article provided herein is formed by a process including the steps of: (a) providing a core including the second agent; (b) heating the compound of formula (A-I) to form a homogenous melt; (c) coating the core with the melt; and (d) cooling the melt to form the article, wherein the compound of formula (A-I) is formulated for sustained release and the second agent is formulated for delayed release. Optionally, the coating includes the compound of formula (A-I) or a binding agent.

In some embodiments, an article provided herein is formed by a process including the steps of: (a) providing a core including the second agent; (b) dissolving the compound of formula (A-I) in a solvent to form a solution; (c) coating the core with the solution; and (d) evaporating the solvent to form the article, wherein the compound of formula (A-I) is formulated for sustained release and the second agent is formulated for delayed release. Optionally, the core includes the compound of formula (A-I) or a binding agent.

In some embodiments, the core includes an admixture of the compound of formula (A-I) and the second agent.

The process for making the articles can include the steps of (i) forming a glassy state composition; and (ii) heating the glassy state composition to a temperature above the glass transition temperature and shaping the glassy state composition to form a shaped article.

In some embodiments, provided herein is a method for making an article, wherein the article is formed by a method including the steps of (i) heat processing a material including the compound of formula (A-I) and the second agent; and (ii) molding, injection molding, extruding, 3D printing, melt electrospinning, fiber spinning, fiber extruding, and/or blow molding the material to form the article.

In some embodiments, provided herein is a method for making an article, wherein the article is formed by a method including the steps of (i) solvent processing a solution including the compound of formula (A-I) and the second agent; and (ii) coating, micro printing, emulsion processing, dot printing, micropatterning, fiber spinning, solvent blow molding, electrospraying, and/or electrospinning the solution to form the article.

In some embodiments, provided herein is a method for making a layered article, wherein the article includes at least two layers (e.g., three layers, four layers, five layers, or more).

In some embodiments, provided herein is a method for making a layered article, wherein the article includes at least two layers (e.g., three layers, four layers, five layers, or more), the method including (i) forming a first layer; and (ii) depositing a second layer atop the first layer.

In some embodiments, provided herein is a method for making a layered article, wherein the article includes a first layer and a second layer, the method including (i) forming the first layer; and (ii) depositing the second layer atop the first layer.

In some embodiments, provided herein is a method for making a layered article, wherein the article includes a first layer and a second layer, the method comprising co-extruding the first layer with the second layer to form the layered article.

### Definitions

The term “free of controlled release polymer,” as used herein, generally refers to the absence (e.g., less than 5 wt. %, less than 2 wt. %, less than 1 wt. %) of an amount of a polymeric material of greater than 10 KDa in the articles provided herein that is sufficient to delay or slow the release of the steroid dimer from the article (e.g., in comparison to the release profile observed for an otherwise identical article containing none of the polymeric material, where the release profile is measured at 37° C. in 100% fetal bovine serum (FBS)).

The term “anti-angiogenic steroid” generally refers to a steroid that halts the process of developing new blood vessels (e.g., angiogenesis). Examples of anti-angiogenic steroids include anecortave acetate, anecortave, 11-epicortisol, 17α-hydroxyprogesterone, tetrahydrocortexolone, and tetrahydrocortisol.

The term “benign steroid” as used herein, generally refers to low glucocorticoid activity and low mineralcorticoid activity. Benign steroids include, without limitation, cholesterol, bile acids (such as cholic acid), and phytosterols (such as beta-sitosterol). Exemplary benign steroids include cholesterol, 11-deoxycortisol, 11-deoxycorticosterone, pregnenolone, cholic acid, chenodeoxycholic acid, ursodeoxycholic acid, obeticholic acid, tetrahydrocortisone, tetra hydrodeoxycortisol, tetra hydrocorticosterone, 5α-dihydrocorticosterone, and 5α-dihydropregesterone.

The term “cholesterol-derivative” generally refers to steroids that are derived from cholesterol. Examples of cholesterol-derivatives are 22R-hydroxycholesterol, and 20α-22R-di hydroxycholesterol.

The term “cholic acid-related bile acid steroid” generally refers to a steroid that is derived from cholic acid. Examples of cholic acid-related bile acid steroids are deoxycholic acid, apocholic acid, dehydrocholic acid, glycochenodeoxycholic acid, glycocholic acid, glycodeoxycholic acid, hyodeoxycholic acid, lithocholic acid, α-muricholic acid, β-muricholic acid, γ-muricholic acid, ω-muricholic acid, taurochenodeoxycholic acid, taurocholic acid, taurodeoxycholic acid, taurolithocholic acid, and tauroursodeoxycholic acid.

The term “cylinder,” as used herein, generally refers to the shape of the pharmaceutical compositions provided herein that has parallel sides and a circular or oval cross section, or a shaped cross section (e.g., a star shaped cross section). A mean diameter of the cylinder can range from about 0.01 to 1 mm diameter, e.g., about 0.01 to 0.2 mm, about 0.1 to 0.3 mm, about 0.1 to 0.4 mm, about 0.2 to 0.5 mm, about 0.1 to 0.6 mm, about 0.1 to 0.7 mm, about 0.1 to 0.8 mm, or about 0.1 to 0.9 mm. A mean length of the cylinder can range from about 0.05 to 20 mm, e.g., about 0.05 to 1 mm, about 0.5 to 2 mm, about 0.5 to 4 mm, about 0.5 to 6 mm, about 0.5 to 8 mm, about 0.5 to 10 mm, about 0.5 to 12 mm, about 0.5 to 14 mm, about 0.5 to 16 mm, or about 0.5 to 18 mm. In some embodiments, the mean diameter of the cylinder is in the range of about 0.01 to 1 mm and the mean length of the cylinder is about 0.1 mm to 4.0 mm. In some embodiments, the mean length of the cylinder is about 0.5 to 10 mm, or about 1 to 10 mm.

The term “fiber,” as used herein, generally refers to the shape of the pharmaceutical compositions provided herein that is elongated or threadlike. A mean diameter of the fiber can range from about 0.01 to 1 mm, e.g., 0.05 to 0.3 mm, 0.1 to 0.3 mm, 0.15 to 0.3 mm, 0.2 to 0.3 mm, 0.25 to 0.3 mm, 0.01 to 0.1 mm, 0.01 to 0.2 mm, 0.01 to 0.3 mm, 0.01 to 0.4 mm, 0.01 to 0.5 mm, 0.01 to 0.6 mm, 0.01 to 0.7 mm, 0.01 to 0.8 mm, or 0.01 to 0.9 mm. A mean length of the fiber can range from about 20 to 20,000 mm, e.g., about 20 to 1000 mm, about 20 to 2,000 mm, about 100 to 2,000 mm, about 100 to 5,000 mm, about 1,000 to 8,000 mm, about 2,000 to 8,000 mm, about 2,000 to 10,000 mm, about 2,000 to 12,000 mm, about 2,000 to 15,000 mm, or about 5,000 to 18,000 mm.

The term “fiber mesh,” as used herein, generally refers to a web or a net in having attached or woven fibers. The fiber mesh can have aligned and unaligned morphologies.

The term “glassy state,” as used herein, generally refers to an amorphous solid including greater than 70%, 80%, 90%, 95%, 98%, or 99% (w/w) of one or more drug dimers provided herein and exhibiting a glass transition temperature greater than 38° C. In some embodiments, the glass transition temperature is in the range of from 38° C. to 150° C. In some embodiments, the glassy state temperature of a compound described herein exhibits a glass transition temperature of greater or equal to 38° C. In some embodiments, the glassy state temperature of a compound described herein exhibits a glass transition temperature of greater or equal to 150° C. In the glassy state, as measured by DSC or XRD, the level of crystallinity is low, ranging from 0-15%, e.g., 0-1%, 0-3%, 0-5%, 0-7%, 0-9%, 0-10%, or 0-13%. Glass formulations provided herein can be formed using heat processing or solvent processing one or more drug dimers.

The term “intraocular pressure (IOP) lowering steroid” generally refers to a steroid that lowers the intraocular pressure of an individual. Examples of intraocular pressure (IOP) lowering steroids are anecortave acetate, anecortave, 11-epicortisol, 17α-hydroxyprogesterone, tetrahydrocortexolone, and tetrahydrocortisol.

The term “microparticle,” as used herein, generally refers to the shape of the pharmaceutical compositions provided herein, which can be regularly or irregularly shaped. A mean diameter of the microparticle can range from about 1 to 1000 μm, e.g., about 10 to 1000 μm, about 100 to 1000 μm, about 200 to 1000 μm, about 500 to 1000 μm, about 700 to 1000 μm, or about 900 to 1000 μm. As used herein, a “microbead” refers to a microparticle that is spherical.

The term “nanoparticle,” as used herein, generally refers to the shape of the pharmaceutical compositions provided herein, which can be regularly or irregularly shaped. A mean diameter of the nanoparticle can range from about 0.01 to 1 μm, e.g., about 0.05 to 1 μm, about 0.1 to 1 μm, about 0.2 to 1 μm, about 0.3 to 1 μm, about 0.4 to 1 μm, about 0.5 to 1 μm, about 0.6 to 1 μm, about 0.7 to 1 μm, about 0.8 to 1 μm, or about 0.9 to 1 μm. As used herein, a “nanobead” refers to a nanoparticle that is spherical.

The term “neurosteroid” generally refers to an endogenous or exogenous steroid that rapidly alters neuronal excitability through interaction with ligand-gated ion channels and other cell surface receptors. Exemplary neurosteroids include, but are not limited to, alphaxalone, alphadolone, hydroxydione, minaxolone, tetrahydrodeoxycorticosterone, allopregnanolone, pregnanolone, ganoxolone, 3α-androstanediol, epipregnanolone, isopregnanolone, and 24(S)-hydroxycholesterol.

The term “non-woven fabric,” as used herein, generally refers to a web structure bonded together by entangling fibers.

The term “other steroid” generally refers to a compound that has a steroid-based structure. Examples of other steroids are flugestone, prebediolone, chlormadinone acetate, medrogestone, and segesterone acetate.

The term “pellet,” as used herein, generally refers to the shape of the pharmaceutical compositions provided herein that is rounded, spherical, or cylindrical, or a combination thereof. A mean diameter of the pellet can range from about 0.2 to 5 mm, e.g., from about 0.2 to 1 mm, from about 0.2 to 2 mm, from about 0.3 to 3 mm, from about 1.5 to 5 mm, from about 2 to 5 mm, from about 2.5 to 5 mm, from about 3 to 5 mm, from about 3.5 to 5 mm, from about 4 to 5 mm, or from about 4.5 to 5 mm.

The term “pharmaceutically acceptable salt” as used herein, represents those salts which are, within the scope of sound medical judgment, suitable for use in contact with the tissues of humans and animals without undue toxicity, irritation, allergic response and the like and are commensurate with a reasonable benefit/risk ratio. Pharmaceutically acceptable salts are well known in the art. For example, S. M. Berge et al. describe pharmaceutically acceptable salts in detail in J. Pharm. Sci. 66:1-19, 1977. The salts can be prepared in situ during the final isolation and purification of the compounds provided herein or separately by reacting the free base group with a suitable organic acid. Representative acid addition salts include acetate, adipate, alginate, ascorbate, aspartate, benzenesulfonate, benzoate, bisulfate, borate, butyrate, camphorate, cam phersulfonate, carbonate, chloride, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like.

The term “pheromone” generally refers to a steroid hormone. Examples of pheromones are androstadienol, androstadienone, androstenol, androstenone, estratetraenol, 5-dehydroprogesterone, 6-dehydro-retroprogesterone, allopregnanolone, and hydroxyprogesterone caproate.

The term “steroid metabolite” generally refers to a product of metabolism of a steroid. Examples of steroid metabolites are tetrahydrotriamcinolone, cortienic acid, 11-dehydrocorticosterone, 11β-hydroxypregnenolone, ketoprogesterone, 17-hydroxypregnenolone, 17,21-dihydroxypregnenolone, 18-hydroxycorticosterone, deoxycortisone, 21-hydroxypregnenolone, and progesterone.

The term “progestin” generally refers to a natural or synthetic steroid hormone. Examples of progestins are allopregnone-3α,20α-diol, allopregnone-3β,20β-diol, allopregnane-3β,21-diol-11,20-dione, allopregnane-3β,17α-diol-20-one, 3,20-allopregnanedione, 3β,11β,17α, 20β,21-pentol, allopregnane-3β,17α,20β,21-tetrol, allopregnane-3α,11β,17α,21-tetrol-20-one, allopregnane-3β,11β,17α,21-tetrol-20-one, allopregnane-3β,17α,20β-triol, allopregnane-3β,17α,21-triol-11,20-dione, allopregnane-3β,11β,21-triol-20-one, allopregnane-3β,17α,21-triol-20-one, allopregnane-3α-ol-20-one, allopregnane-3β-ol-20-one, pregnanediol, 3,20-pregnanedione, 4-pregnene-20,21-diol-3,11-dione, 4-pregnene-11β,17α,20β,21-tetrol-3-one, 4-pregnene-17α,20β,21-triol-3,11-dione, 4-pregnene-17α,20β,21-triol-3-one, and pregnenolone.

The term “surface erosion,” as used herein, generally refers to a process of a gradual disintegration or dissolution of the articles or pharmaceutical compositions provided herein. In some embodiments, surface erosion is a measure of the release of a free drug from the drug dimer. Surface erosion can be tailored to achieve desired drug release rates. Surface erosion can depend on the drug composition of the drug dimer, and can be modulated by the cleavage of drug-linker bond through hydrolysis and/or enzymatic degradation. The rate of surface erosion and release of a given drug from a drug dimer may also depend on the quantity of the loaded drug dimer as a percent of the final drug dimer formulation, article size (e.g. dimensions), solubility of drug dimer (e.g., through selection of appropriate drug and/or linker), and/or surface area of the article. For example, surface erosion mechanism of drug release allows drug delivery articles to be tailored with specific physical features (dimensions, diameters, surface areas, total mass, etc.) to achieve desired drug release rates, and drug release may be designed to be initiated within minutes or hours, and may continue to occur over days, weeks, months, or years post implantation.

As stated herein, “t₅₀” is the time at which 50% of the releasable drug has been released from an article provided herein. For example, time t₁₀ is, correspondingly, the time at which 10% of the releasable drug has been released from an article provided herein. In some embodiments, when the release curve is linear, t₁₀=⅕ of t₅₀. In some embodiments, when there is an initial burst of released drug, t₁₀ is much less than ⅕° t₅₀. In the compositions and methods provided herein t₁₀ can be equal to or greater than 1/10 of t₅₀. Drug release from an article or compound provided herein can be measured at 37° C. in 100% bovine serum or at 37° C. in phosphate buffered saline (PBS).

By “sustained release” is generally meant the release profile of a drug (e.g., D1, D2, or the second agent) from an article provided herein in phosphate buffered saline (PBS) (e.g., at 37° C. is such that less than 20% of the drug is released from the article over a period of 2 days).

By “immediate release” is generally meant the release profile of the second agent from an article provided herein in phosphate buffered saline (PBS) (e.g., at 37° C. is such that greater than 50% of the second agent is released from the article over a period of 2 days).

By “delayed release” is generally meant the release profile of the second agent from an article provided herein in phosphate buffered saline (PBS) (e.g., at 37° C. is such that less than 10% (w/w) of the second agent released prior to the release of at least 50% (w/w) of the compound of formula (A-I) from the article).

The term “woven fabric,” as used herein, generally refers to pharmaceutical compositions that resemble materials that are formed by weaving of fibers.

### Chemical Definitions

By “acyl” is meant a chemical moiety with the formula —C(O)R′, where R′ is selected from the group consisting of C₁₋₁₀ alkyl, C₂₋₂₀ alkene, heteroalkyl, C₂₋₂₀ alkyne, C₅₋₁₀ aryl, and cyclic system. Examples of acyl groups include, without limitation, acetyl, propanoyl, butanoyl, pentanoyl, and tetra hydrofuran-2-oyl.

By “aliphatic” is meant a non-aromatic chemical moiety of hydrocarbons. Aliphatics may be cyclic, straight, or branched chains, and may be saturated or unsaturated, and may have single, double, or triple bonds.

By “alkoxy” is meant a chemical substituent of the formula —OR, wherein R is an alkyl group. By “aryloxy” is meant a chemical substituent of the formula —OR, wherein R is a C₅₋₁₀ aryl group.

As used herein, the terms “alkylene,” “alkenylene,” “alkynylene,” and the prefix “alk” refer to divalent groups having a specified size, typically C₁₋₁₀ or C₁₋₂₀ for the saturated groups (e.g., alkylene or alk) and C₂₋₂₀ or C₂₋₂₀ for the unsaturated groups (e.g., alkenylene or alkynylene). They include straight-chain, branched-chain, and cyclic forms as well as combinations of these, containing only C and H when unsubstituted. Because they are divalent, they can link together two parts of a molecule. Examples are methylene, ethylene, propylene, cyclopropan-1,1-diyl, ethylidene, 2-butene-1,4-diyl, and the like. These groups can be substituted by the groups typically suitable as substituents for alkyl, alkenyl and alkynyl groups as set forth herein. Thus C═O is a C1 alkylene that is substituted by ═O, for example.

By “alkylthio” is meant a chemical substituent of the formula —SR, wherein R is an alkyl group.

By “arylthio” is meant a chemical substituent of the formula —SR, wherein R is a C₅₋₁₀ aryl group.

By “C₁₋₂₀ alkyl” is meant a branched or unbranched saturated hydrocarbon group, having 1 to 20 carbon atoms, inclusive. An alkyl may optionally include monocyclic, bicyclic, or tricyclic rings, in which each ring desirably has three to six members. The alkyl group may be substituted or unsubstituted. Exemplary substituents include alkoxy, aryloxy, sulfhydryl, alkylthio, arylthio, halogen, hydroxyl, fluoroalkyl, perfluoralkyl, amino, aminoalkyl, disubstituted amino, quaternary amino, hydroxyalkyl, carboxyalkyl, and carboxyl groups.

By “C₂₋₂₀ alkene” is meant a branched or unbranched hydrocarbon group containing one or more double bonds, desirably having from 2 to 10 carbon atoms. A C₂₋₂₀ alkene may optionally include monocyclic, bicyclic, or tricyclic rings, in which each ring desirably has five or six members. The C₂₋₂₀ alkene group may be substituted or unsubstituted. Exemplary substituents include alkoxy, aryloxy, sulfhydryl, alkylthio, arylthio, halogen, hydroxyl, fluoroalkyl, perfluoralkyl, amino, aminoalkyl, disubstituted amino, quaternary amino, hydroxyalkyl, carboxyalkyl, and carboxyl groups.

By “C₂₋₂₀ alkyne” is meant a branched or unbranched hydrocarbon group containing one or more triple bonds, desirably having from 2 to 10 carbon atoms. A C₂₋₂₀ alkyne may optionally include monocyclic, bicyclic, or tricyclic rings, in which each ring desirably has five or six members. The C₂₋₂₀ alkyne group may be substituted or unsubstituted. Exemplary substituents include alkoxy, aryloxy, sulfhydryl, alkylthio, arylthio, halogen, hydroxyl, fluoroalkyl, perfluoralkyl, amino, aminoalkyl, disubstituted amino, quaternary amino, hydroxyalkyl, carboxyalkyl, and carboxyl groups.

By “carbonate ester” is meant a linkage group having the formula —C(O)O—C(O)—O—.

By “carboxyalkyl” is meant a chemical moiety with the formula —(R)—COOH, wherein R is an alkyl group.

By “cyclic acetal” is meant a ring structure including two oxygen atoms separated by a carbon atom which is optionally substituted (e.g., 1,3-dioxolane). Exemplary substituents include, without limitation, alkyl, hydroxyl, alkoxy, aryloxy, sulfhydryl, alkylthio, arylthio, halogen, fluoroalkyl, carboxyl, carboxyalkyl, amino, aminoalkyl, monosubstituted amino, disubstituted amino, quaternary amino, phosphodiester, phosphoramidate, phosphate, phosphonate, phosphonate ester, sulfonate, sulfate, sulfhydryl, phenol, amidine, guanidine, and imidazole groups.

The term “cyclic system” refers to a compound that contains one or more covalently closed ring structures, in which the atoms forming the backbone of the ring are composed of any combination of the following: carbon, oxygen, nitrogen, sulfur, and phosphorous. The cyclic system may be substituted or unsubstituted. Exemplary substituents include, without limitation, alkyl, hydroxyl, alkoxy, aryloxy, sulfhydryl, alkylthio, arylthio, halogen, fluoroalkyl, carboxyl, carboxyalkyl, amino, aminoalkyl, monosubstituted amino, disubstituted amino, and quaternary amino groups.

By “fluoroalkyl” is meant an alkyl group that is substituted with a fluorine.

By “heteroalkyl” is meant a branched or unbranched alkyl group in which one or more methylenes (—CH₂—) are replaced by nitrogen, oxygen, sulfur, carbonyl, thiocarbonyl, phosphoryl, or sulfonyl moieties. Some examples include tertiary amines, ethers, thioethers, amides, thioamides, carbamates, thiocarbamates, phosphoramidates, sulfonamides, and disulfides. A heteroalkyl may optionally include monocyclic, bicyclic, or tricyclic rings, in which each ring desirably has three to six members. The heteroalkyl group may be substituted or unsubstituted. Exemplary substituents include alkoxy, aryloxy, sulfhydryl, alkylthio, arylthio, halogen, hydroxyl, fluoroalkyl, perfluoralkyl, amino, aminoalkyl, disubstituted amino, quaternary amino, hydroxyalkyl, carboxyalkyl, and carboxyl groups.

By “hydroxyalkyl” is meant a chemical moiety with the formula —(R)—OH, wherein R is an alkyl group.

### BRIEF DESCRIPTION OF THE DRAWINGS

FIG. 1A-1C shows the chemical structure of an exemplary composition provided herein (e.g., Compound 1 (Dex-TEG-Dex)) and processed into pellets and films.

FIG. 2 shows a graph of dexamethasone release from pellets of Compound 1

FIG. 3A-3D shows optical microscope images of exemplary compositions provided herein (e.g., Compound 1 and paclitaxel combinations) in different physical forms and ratios.

FIG. 4A-4B shows drug release from films and pellets of exemplary compositions provided herein (e.g., Compound 1 and paclitaxel) in PBS at 37° C.

FIG. 5A-5D shows optical microscope images of exemplary compositions provided herein (e.g., Compound 1 and lidocaine combinations) in different physical forms and ratios.

FIG. 6A-6B shows drug release from films and pellets of exemplary compositions provided herein (e.g., Compound 1 and lidocaine) in PBS at 37° C.

FIG. 7A-7C shows optical microscope images of exemplary compositions provided herein (e.g., Compound 1 and ciprofloxacin HCl combinations) in different physical forms and ratios.

FIG. 8A-8B shows drug release from films and pellets of exemplary compositions provided herein (e.g., Compound 1 and ciprofloxacin HCl combinations) in PBS at 37° C.

FIG. 9A-9B shows optical microscope images of exemplary compositions provided herein (e.g., compound 1 and sunitinib malate pellets) in different combination ratios.

FIG. 10A-10B shows drug release from pellets of exemplary compositions provided herein (e.g., Compound 1 and sunitinib malate) in PBS at 37° C.

FIG. 11A-11B shows optical microscope images of exemplary compositions provided herein (e.g., Compound 1 and travoprost films) in different combination ratios.

FIG. 12A-12B shows drug release from films of exemplary compositions provided herein (e.g., Compound 1 and travoprost) in PBS at 37° C.

FIG. 13 shows a composition provided herein (e.g., Compound 1 and chlorhexidine diacetate) coated on a titanium (Ti) film at 2:1 ratio

FIG. 14 shows drug release in PBS at 37° C. from the article of FIG. 13 (e.g., Compound 1 and chlorhexidine diacetate coating on Ti film at 2:1 ratio).

FIG. 15A-15E shows optical microscope images of fibers formed from exemplary compositions provided herein (e.g., compound 1 and different secondary agents including lidocaine, ciprofloxacin HCl, sunitinib malate, and travoprost).

FIG. 16A-16C shows the chemical structure of Compound 2 (HC-TEG-HC) and drug release from exemplary compositions provided herein (e.g., Compound 2 and Ibuprofen and Naproxen) coated on glass (e.g., at 10:1 ratio in PBS at 37° C.).

FIG. 17A-17E shows the chemical structure of Compound 3 (CHS-TEG-CHS) and optical microscope images of solvent cast films of exemplary compositions provided herein (e.g., Compound 3 and the secondary agents, for example, lidocaine or paclitaxel) in different ratios.

FIG. 18A-18D shows optical microscope images of exemplary compositions provided herein (e.g., Compound 1 and spironolactone pellets) in different combination ratios.

FIG. 19A-19B shows drug release from pellets of exemplary compositions provided herein (e.g., Compound 1 and spironolactone) in PBS at 37° C.

FIG. 20A-20E shows the chemical structure of Compound 4 (Pred-TEG-Pred) and optical microscope images of exemplary compositions provided herein (e.g., Compound 4 and Diclofenac, Diclofenac Sodium Salt, and Flurbiprofen pellets) in different combination ratios.

FIG. 21A-21B shows drug release from pellets of exemplary compositions provided herein (e.g., Compound 4 and Diclofenac, Diclofenac Sodium Salt, and Flurbiprofen) in PBS at 37° C.

### DETAILED DESCRIPTION

For locally administered sustained release delivery systems, challenges can arise where the mass balance of the carrier or matrix for the drug hinders drug loading, or where the carriers and matrices produce unwanted effects (e.g., such as local inflammation).

There is an unmet need for a sustained release drug system capable of delivering multiple drugs simultaneously in the absence or with a minimal amount of carrier and/or excipient agents, at a rate-controlled manner over an extended period of time (e.g., days to weeks, to months or even years), where the system contains predominantly the combination of drugs being delivered, and the system minimizes side effects associated with the use of carriers or matrices.

Provided herein are articles formed from a steroid dimer that control the release profile of a second agent contained on or within the article. The release rate of the second agent from an article provided herein can be controlled through several engineerable design parameters, including, but not limited to: 1) selection of the steroid drug; 2) selection of the functional group of the drug for conjugation (e.g., if multiple exist); 3) selection of the linker; 4) selection of the linkage group (e.g., esters, carbonates, carbonate esters, or anhydrides); 5) selection of the surface area of the shaped article; 6) the location of the second agent (e.g., a homogenous mixture with the steroid dimer or positioned in a layer on or in the article); and 7) the extent of loading of the second agent in the article. In some embodiments, the articles and compositions provided herein comprise different release profiles of the steroids used in the dimer by using the free steroid (not conjugated D1 or D2) as the second agent (e.g., the articles of the invention where the second agent is a free steroid drug). In some embodiments, the articles and compositions provided herein comprise the controlled release of two or more steroid drugs through the use of heterodimers (e.g., different steroid drugs on the two ends of the linkers) in combination with a second agent, or by forming shaped articles with mixtures of two or more steroid homodimers, steroid heterodimers, or a mixture of both steroid homodimers and steroid heterodimers in combination with a second agent, or by using a steroid as the second agent that is different from the steroid drug(s) used in the dimer. For example, the articles provided herein can produce a sustained release of the second agent, without exhibiting any burst release (e.g., t₁₀ can be equal to or greater than 1/10 of t₅₀) and without reliance upon degradable matrices (e.g. controlled release polymers), which can cause undesirable local side effects (such as inflammation), when, e.g., the second agent is distributed (heterogeneously or homogenously) in the steroid dimer matrix. Alternatively, the articles provided herein can produce an immediate release of the second agent when, e.g., the second agent resides in a layer on the surface of a core formed from the steroid dimer, where the core is free of the second agent. In some embodiments, the layer including the second agent further includes a binding agent and/or the layer including the second agent further includes the steroid dimer. In some embodiments, the articles provided herein produce a delayed release of the second agent when, e.g., the steroid dimer resides on the surface of a core formed from the second agent. In some embodiments, the core formed from the second agent further includes a binding agent or the steroid dimer. In some embodiments, the high drug loading of the articles provided herein are suitable for producing locally effective concentrations of the second agent for periods of days to weeks to months or even years.

### Steroid Dimers

The disclosure features compounds of formula (A-VIII) and articles formed from compounds of formula (A-VIII):

D1-L-D2 (A-VIII)

or a pharmaceutically acceptable salt thereof, wherein each of D1 and D2 is, independently, a radical formed from a steroid; and L is a linker covalently linking D1 to D2. Each of D1 and D2 can be, independently, selected from an anabolic steroid, an androgenic steroid, a progestin steroid, an estrogen steroid, a cancer treatment steroid, an antibiotic steroid, a glucocorticoid steroid, a benign steroid, or a corticosteroid. L can be covalently linked to D1 and to D2 via one or more ester, carbonate, carbonate ester, or anhydride linkages. Ester, carbonate, carbonate ester, or anhydride linkages formed from a functional group on D1 and D2 can be selected from, e.g., hydroxyl or carboxy. For example, L can include the radical —C(O)—(R^{A})—C(O)—, —C(O)—OC(O)—(R^{A})—C(O)O—C(O)—, or —O—(R^{A})—O—, where R^{A} is a radical of a polyol and includes at least one free hydroxyl group or R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, —(CH₂CH₂O)_{q}CH₂CH₂—, —(CH₂CH₂CH₂CH₂O)ᵣCH₂CH₂CH₂CH₂—, or —(CH₂CH(CH₃)O)ₛCH₂CH(CH₃)—, and q, r, and s are integers from 1 to 10 (e.g., 1 to 10, 1 to 5, or 5 to 10). The articles provided herein can be machined, molded, emulsion-processed, electrospun, electrosprayed, blow molded, dry spun, heat spun, melt spun, gel spun, or extruded to form a fiber, fiber mesh, woven fabric, non-woven fabric, film, pellet, cylinder, microparticle (e.g., a microbead), nanoparticle (e.g., a nanobead), or another shaped article.

The compound can be further described by the formula (A-II):

D1-O-L-O-D2 (A-II),

or a pharmaceutically acceptable salt thereof, wherein each of D1-O and D2-O is, independently, a radical formed from a steroid.

In some embodiments, each of D1-O and D2-O is, independently, described by any one of formulas (I-a) to (I-zzz):

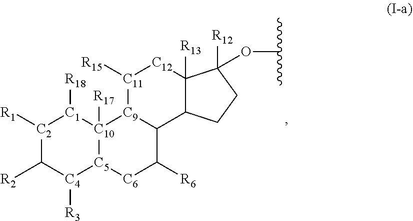

where the bond between C₁ and C₂, C₄ and C₅, C₅ and C₆, C₉ and C₁₀, and C₁₁ and C₁₂ is a single or a double bond; R₁ represents H, CH₃, or HC(O); R₂ represents ═O, OH, or H; or R₁ and R₂ taken together with carbons to which they are attached form an isoxazole; R₃ represents H, a halogen atom, or OH; R₆ represents H or CH₃; R₁₂ represents H, CH₃, or CH₃CH₂; R₁₃ represents CH₃ or CH₃CH₂; R₁₅ represents H or OH; R₁₇ represents H or CH₃; and R₁₈ represents H or CH₃;

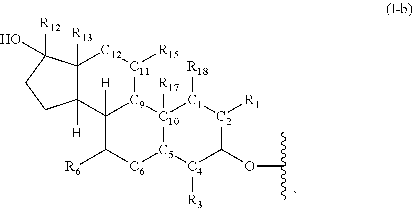

where the bond between C₁ and C₂, C₄ and C₅, C₅ and C₆, C₉ and C₁₀, and CH and C₁₂ is a single or a double bond; R₁ represents H, CH₃, or HC(O); R₃ represents H, a halogen atom, or OH; R₆ represents H or CH₃; R₁₂ represents H, CH₃, or CH₃CH₂; R₁₃ represents CH₃ or CH₃CH₂; R₁₅ represents H or OH; R₁₇ represents H or CH₃; and R₁₈ represents H or CH₃;

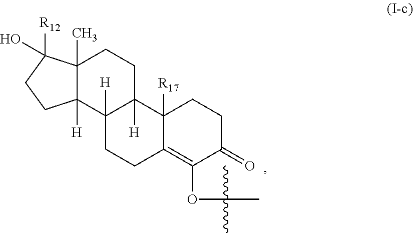

where R₁₂ represents H or CH₃; and R₁₇ represents H or CH₃;

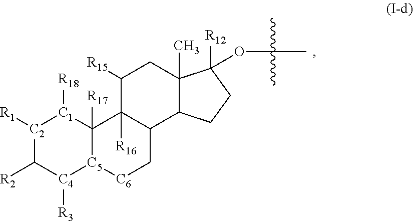

where the bond between C₁ and C₂, C₄ and C₅, and C₅ and C₆ is a single or a double bond; C₂ is O, C or CH₂; R₁ represents H, —CHOH, or is absent; R₂ represents ═O or OH; or R₁ and R₂ taken together with carbons to which they are attached form a pyrazole; R₃ represents H or OH; R₁₂ represents H, CH₃, optionally substituted alkynylene, C₁₋₆ alkoxy, or CH₃CH₂; R₁₅ represents H or OH; R₁₆ represents H or a halogen atom; R₁₇ represents H or CH₃; and R₁₈ represents H or CH₃;

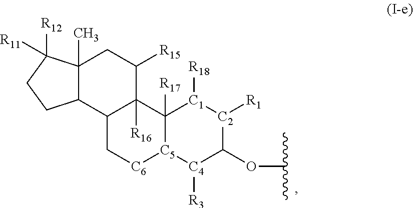

where the bond between C₁ and C₂, C₄ and C₅, and C₅ and C₆ is a single or a double bond; C₂ is O, C or CH₂; R₁ represents H, —CHOH, or is absent; R₃ represents H or OH; R₁₁ represents H, OH, CH₃, optionally substituted alkynylene, CH₃CH₂, ═O, —OC(O)CH₂CH₃, or is absent; R₁₂ represents H, OH, CH₃, optionally substituted alkynylene, CH₃CH₂, ═O, —OC(O)CH₂CH₃, or is absent; R₁₅ represents H or OH; R₁₆ represents H or a halogen atom; R₁₇ represents H or CH₃; and R₁₈ represents H or CH₃;

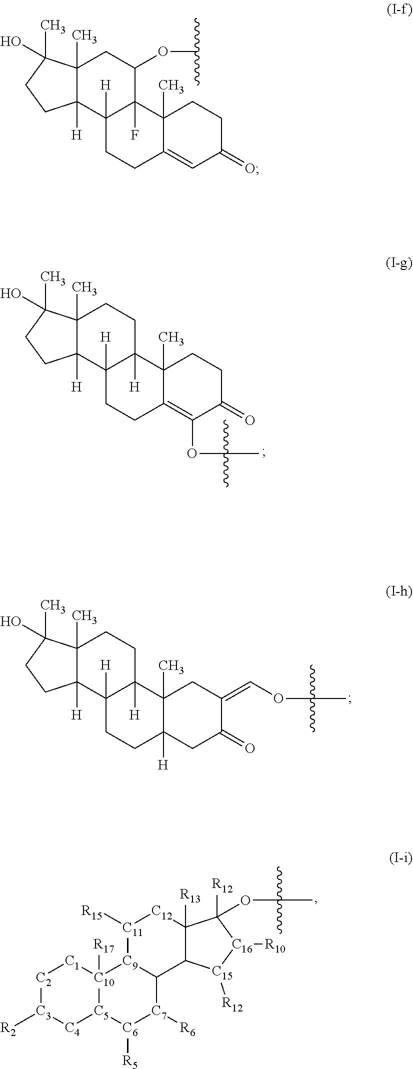

where the bond between C₁ and C₁₀, C₂ and C₃, C₃ and C₄, C₄ and C₅, C₅ and C₆, C₆ and C₇, C₅ and C₁₀, C₉ and C₁₀, C₁₁ and C₁₂, C₁₅ and C₁₆ is a single or a double bond; R₂ represents H, ═O, OH, —NOH, or C₁₋₆ alkoxy; R₅ represents H, CH₃, or a halogen atom; R₆ represents H or CH₃; or R₅ and R₆ taken together with carbons to which they are attached form a cyclopropane; R₉ is H; R₁₀ is H or ═CH₂; or R₉ and R₁₀ taken together with carbons to which they are attached form a cyclopropane; R₁₂ represents H, optionally substituted alkynylene, —CH₂CH═CH₂, CH₃, —C(O)CH₃, or —CH═CH₂; Rn represents CH₃ or CH₂CH₃; R₁₅ represents H or ═CH₂; and R₁₇ represents H, CH₃, or is absent;

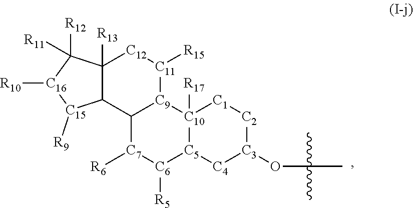

where the bond between C₁ and C₁₀, C₂ and C₃, C₄ and C₅, C₆ and C₇, C₅ and C₁₀, C₉ and C₁₀, C₁₁ and C₁₂, C₁₅ and C₁₆ is a single or a double bond; R₅ represents H, CH₃, or a halogen atom; R₆ represents H or CH₃; or R₅ and R₆ taken together with carbons to which they are attached form a cyclopropane; R₉ is H; R₁₀ is H or ═CH₂; or R₉ and R₁₀ taken together with carbons to which they are attached form a cyclopropane; R¹¹ represents H, OH, optionally substituted alkynylene, —C(O)CH₃, —CH₂CH═CH₂, a halogen atom, —CH═CH₂, —OC(O)CH₃, CH₃, —C(O)C(OH)CH₃; R₁₂ represents H, OH, optionally substituted alkynylene, —C(O)CH₃, —CH₂CH═CH₂, a halogen atom, —CH═CH₂, —OC(O)CH₃, CH₃, —C(O)C(OH)CH₃; or R₁₁ and R₁₂ together with carbon to which they are attached form a lactone; R₁₃ represents CH₃ or CH₂CH₃; R₁₅ represents H or ═CH₂; and R₁₇ represents H, CH₃, or is absent;

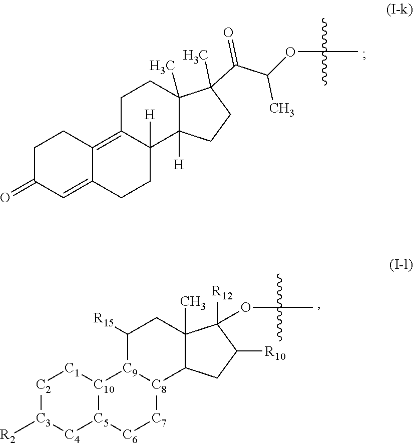

where the bond between C₁ and C₂, C₁ and C₁₀, C₂ and C₃, C₃ and C₄, C₄ and C₅, C₆ and C₇, C₅ and C₁₀, C₇ and C₈, and C₈ and C₉ is a single or a double bond; R₂ represents OH, —OC(O)Ph, or C₁₋₆ alkoxy; R₁₀ represents H or OH; R₁₂ represents H, optionally substituted alkynylene; and R₁₅ represents H or C₁₋₆ alkoxy;

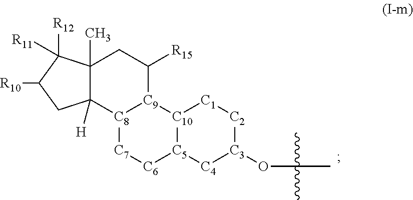

where the bond between C₁ and C₂, C₁ and C₁₀, C₂ and C₃, C₃ and C₄, C₄ and C₅, C₆ and C₇, C₅ and C₁₀, C₇ and C₈, and C₈ and C₉ is a single or a double bond; R₁₀ represents H or OH; R₁₁ represents H, OH, optionally substituted alkynylene, ═O, or is absent; R₁₂ represents H, OH, optionally substituted alkynylene, ═O, or is absent; and R₁₅ represents H or C₁₋₆ alkoxy;

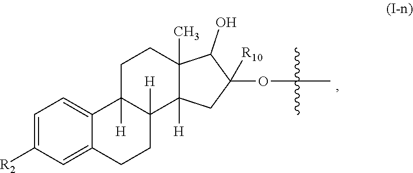

where R₂ represents OH or C₁₋₆ alkoxy; and R₁₀ represents H or CH₃;

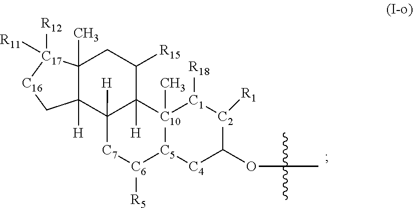

where the bond between C₁ and C₂, C₄ and C₅, C₅ and C₆, C₆ and C₇, and C₁₆ and C₁₇ is a single or a double bond; C₄ is NH, CH, or CH₂; R₁ represents H; R₅ represents H or a halogen atom; R₁₁ represents H, optionally substituted heteroaryl, —C(O)C₁₋₆ alkyl, —C(O)OC₁₋₆ alkyl, or —C(O)NHR, where R is optionally substituted alkyl or aryl; R₁₂ represents H, optionally substituted heteroaryl, —C(O)C₁₋₆ alkyl, —C(O)OC₁₋₆ alkyl, or —C(O)NHR, where R is optionally substituted alkyl or aryl; and R₁₈ represents H; or R₁ and R₁₈ taken together with carbons to which they are attached form a cyclopropane;

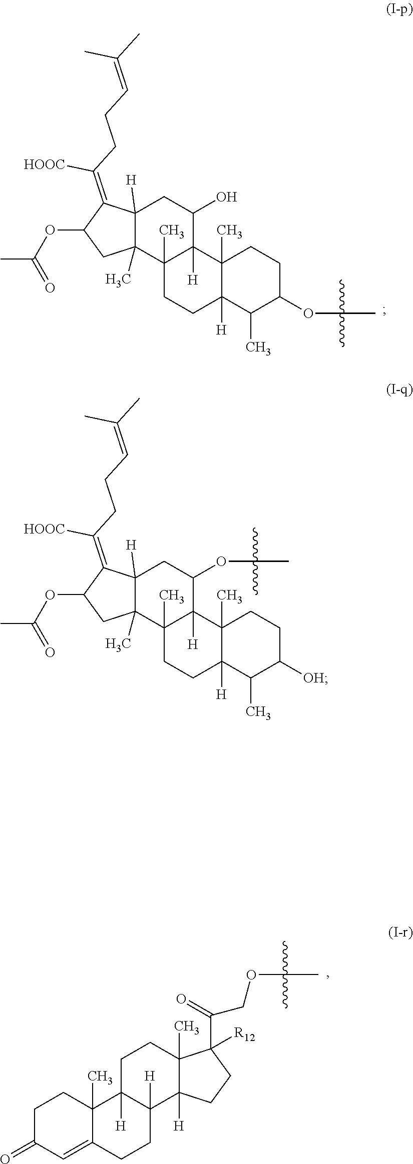

where R₁₂ is H or OH;

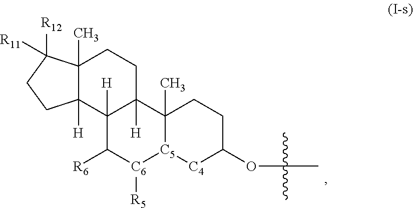

where the bond between C₄ and C₅, and C₅ and C₆ is a single or a double bond; R₅ represents H or C₁₋₆ alkyl; R₆ represents H or OH; R₁₁ represents H, OH, —C(O)C₁₋₆ alkyl, —C(O)CH₂OH, or —CH(CH₃)CH₂CH₂C(O)OH; and R₁₂ represents H, OH, —C(O)C₁₋₆ alkyl, —C(O)CH₂OH, or —CH(CH₃)CH₂CH₂C(O)OH;

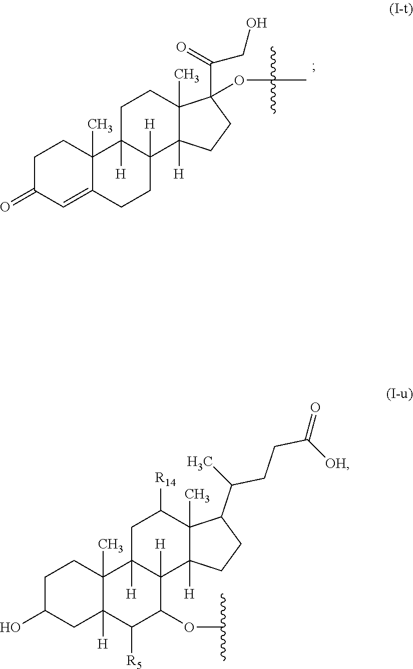

where R₅ represents H or CH₂CH₃; and R₁₄ represents H or OH;

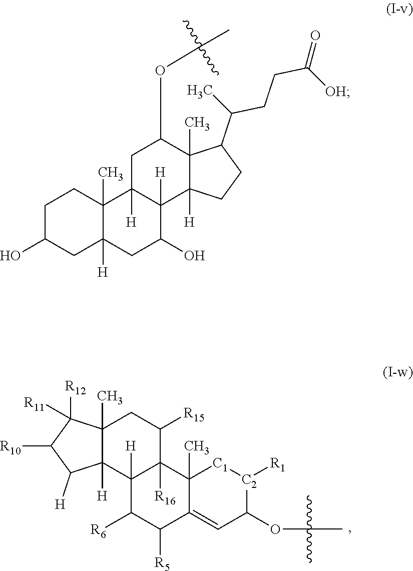

where the bond between C₁ and C₂ is a single or a double bond; R₁ represents H or a halogen atom; R₅ represents H, C₁₋₆ alkyl, or a halogen atom; R₆ represents H or a halogen atom; R₁₀ represents H, C₁₋₆ alkyl, OH, or ═CH₂; R₁₁ represents H, OH, C₁₋₆ alkyl, optionally substituted —C(O)C₁₋₆ alkyl, —C(O)CH₂OC(O)C₁₋₆ alkyl, optionally substituted —OC(O)C₁₋₆ alkyl, —OC(O)Ph, —OC(O)heterocyclyl, —CH₂C(O)CH₂OH, —C(O)C(O)OH, —C(O)C(O)OC₁₋₆ alkyl, —C(O)SCH₂F, or —OC(O)OC₁₋₆ alkyl; or R₁₀ and R₁₁ taken together with carbons to which they are attached form an optionally substituted cyclic acetal or optionally substituted heterocyclyl; R₁₂ represents H, OH, C₁₋₆ alkyl, optionally substituted —C(O)C₁₋₆ alkyl, —C(O)CH₂OC(O)C₁₋₆ alkyl, optionally substituted —OC(O)C₁₋₆ alkyl, —OC(O)Ph, —OC(O)heterocyclyl, —CH₂C(O)CH₂OH, —C(O)C(O)OH, —C(O)C(O)OC₁₋₆ alkyl, —C(O)SCH₂F, or —OC(O)OC₁₋₆ alkyl; or R₁₀ and R₁₂ taken together with carbons to which they are attached form an optionally substituted cyclic acetal or optionally substituted heterocyclyl; R₁₅ represents H, OH, ═O, or a halogen atom; and R₁₆ represents H or a halogen atom;

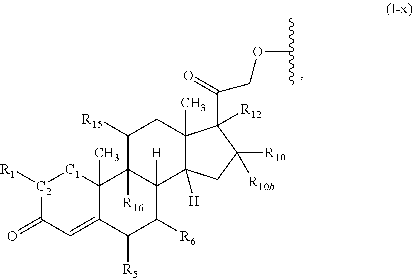

where the bond between C₁ and C₂ is a single or a double bond; R₁ represents H or a halogen atom; R₅ represents H, C₁₋₆ alkyl, or a halogen atom; R₆ represents H or a halogen atom; R₁₀ represents H, C₁₋₆ alkyl, OH, or ═CH₂; REM represents H, C₁₋₆ alkyl, OH, ═CH₂, or be absent; R₁₂ represents H, OH, optionally substituted —C(O)C₁₋₆ alkyl, —C(O)CH₂OC(O)C₁₋₆ alkyl, optionally substituted —OC(O)C₁₋₆ alkyl, or —OC(O)Ph; or R₁₀ and R₁₁ taken together with carbons to which they are attached form an optionally substituted cyclic acetal or optionally substituted heterocyclyl; R₁₅ represents H, OH, ═O, or a halogen atom; and R₁₆ represents H or a halogen atom;

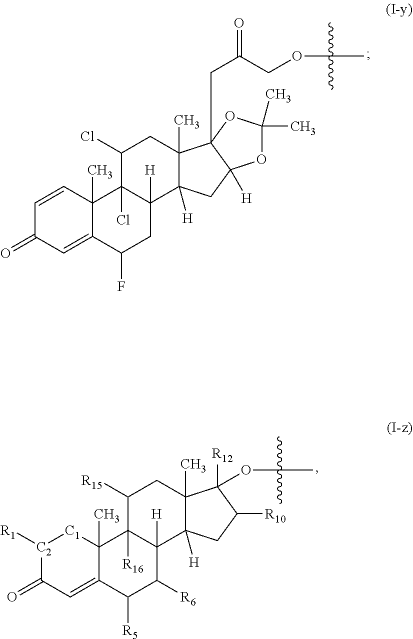

where the bond between C₁ and C₂ is a single or a double bond; R₁ represents H or a halogen atom; R₅ represents H, a halogen atom, or CH₃; R₆ represents H, a halogen atom; R₁₀ represents H, OH, CH₃, or ═CH₂; R₁₂ represents optionally substituted —C(O)C₁₋₆ alkyl, —C(O)CH₂OC(O)C₁₋₆ alkyl, or —C(O)SCH₂F; R₁₅ represents OH or ═O; and R₁₆ represents H or a halogen atom;

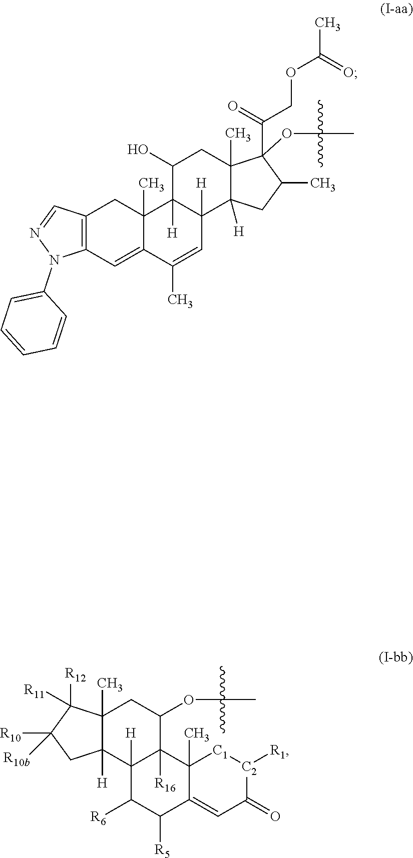

where the bond between C₁ and C₂ is a single or a double bond; R₁ represents H or a halogen atom; R₅ represents H, C₁₋₆ alkyl, or a halogen atom; R₆ represents H or a halogen atom; R₁₀ represents H, C₁₋₆ alkyl, OH, or ═CH₂; R_{10b} represents H, C₁₋₆ alkyl, OH, or ═CH₂, or is absent; R₁₁ represents H, OH, C₁₋₆ alkyl, optionally substituted —C(O)C₁₋₆ alkyl, —C(O)CH₂OC(O)C₁₋₆ alkyl, optionally substituted —OC(O)C₁₋₆ alkyl, —OC(O)Ph, —OC(O)heterocyclyl, —CH₂C(O)CH₂OH, —C(O)C(O)OH, —C(O)C(O)OC₁₋₆ alkyl, —C(O)SCH₂F, or —OC(O)OC₁₋₆ alkyl; or R₁₀ and R₁₁ taken together with carbons to which they are attached form an optionally substituted cyclic acetal or optionally substituted heterocyclyl; R₁₂ represents H, OH, C₁₋₆ alkyl, optionally substituted —C(O)C₁₋₆ alkyl, —C(O)CH₂OC(O)C₁₋₆ alkyl, optionally substituted —OC(O)C₁₋₆ alkyl, —OC(O)Ph, —OC(O)heterocyclyl, —CH₂C(O)CH₂OH, —C(O)C(O)OH, —C(O)C(O)OC₁₋₆ alkyl, —C(O)SCH₂F, or —OC(O)OC₁₋₆ alkyl; or R₁₀ and R₁₂ taken together with carbons to which they are attached form an optionally substituted cyclic acetal or optionally substituted heterocyclyl; and R₁₆ represents H or a halogen atom;

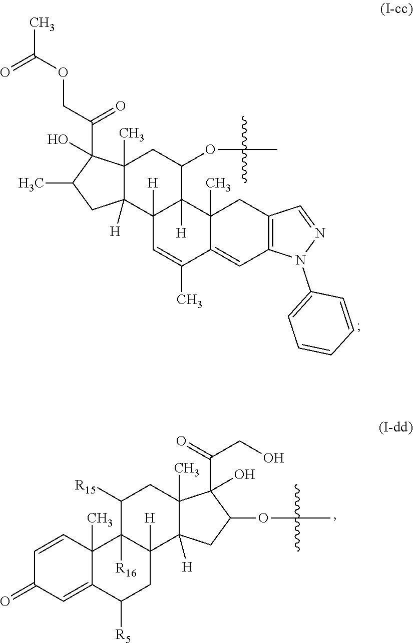

where R₅ represents H or a halogen atom; R₁₅ represents a halogen atom or OH; and R₁₆ represents H or a halogen atom;

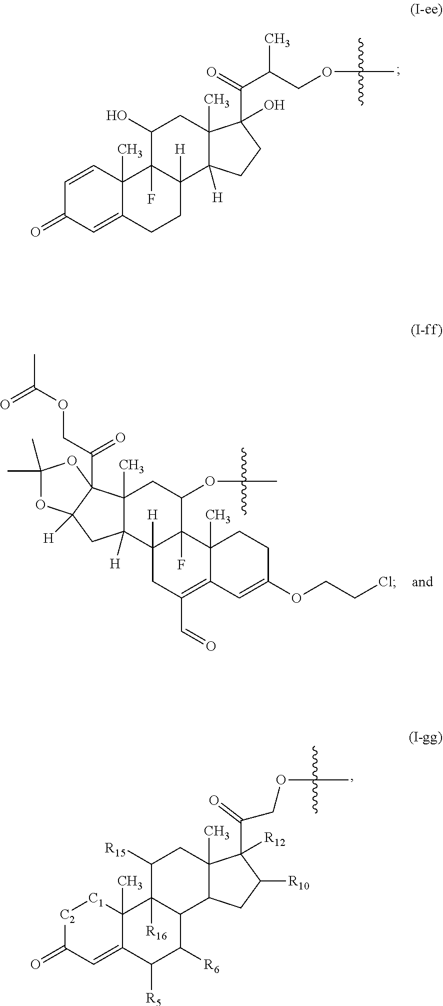

where the bond between C₁ and C₂ is a double or a single bond; R₁₆ represents H or a halogen atom; R₅ represents H, CH₃, or a halogen atom; R₁₂ represents H or a halogen atom; R₁₅ represents ═O or OH; R₁₂ and R₁₀ each, independently, represent —H, C₁₋₁₀ alkyl, —OH, —O-acyl, or R₁₂ and R₁₀ combine to form a cyclic acetal of formula (XVIII-a) where:

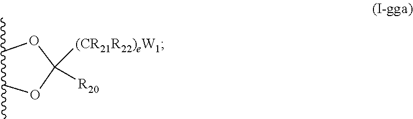

e is an integer from 0 to 6; R₂₀, R₂₁, and R₂₂ each, independently, represent H or C₁₋₁₀ alkyl; and W₁ represents H or CH₃;

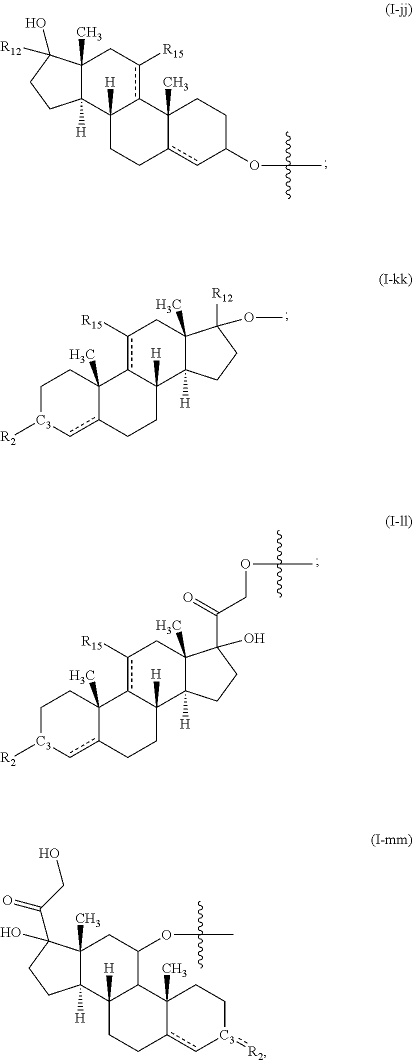

where the bond between C₃ and R₂ is a single or a double bond; R₂ represents OH or ═O; R₁₂ represents —C(═O)CH₂OC(═O)CH₃, —C(═O)CH₂OH, or —C(═O)CH₃; R₁₅ represents H or OH;

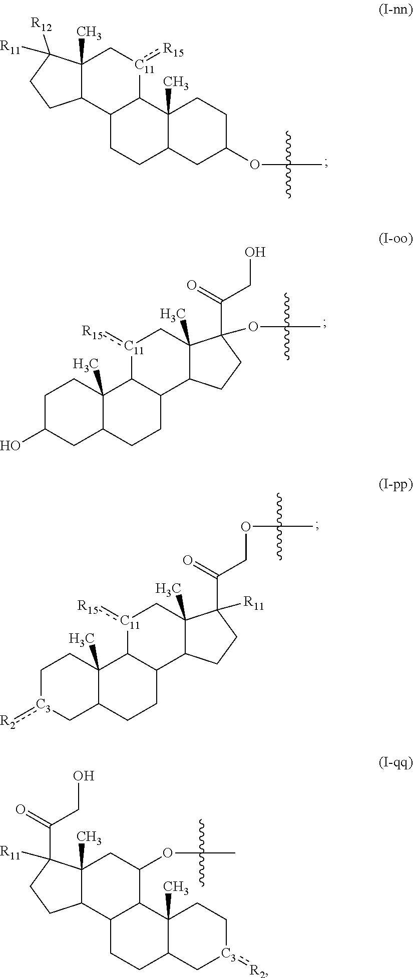

where the bond between C₃ and R₂, and C_{H} and R₁₅ is a single or a double bond; R₂ represents OH or ═O; R₁₁ represents H, OH, —C(═O)CH₂OH, or —C(═O)CH₃; R₁₂ represents H, OH, —C(═O)CH₂OH, or —C(═O)CH₃; R₁₅ represents H, ═O, or OH;

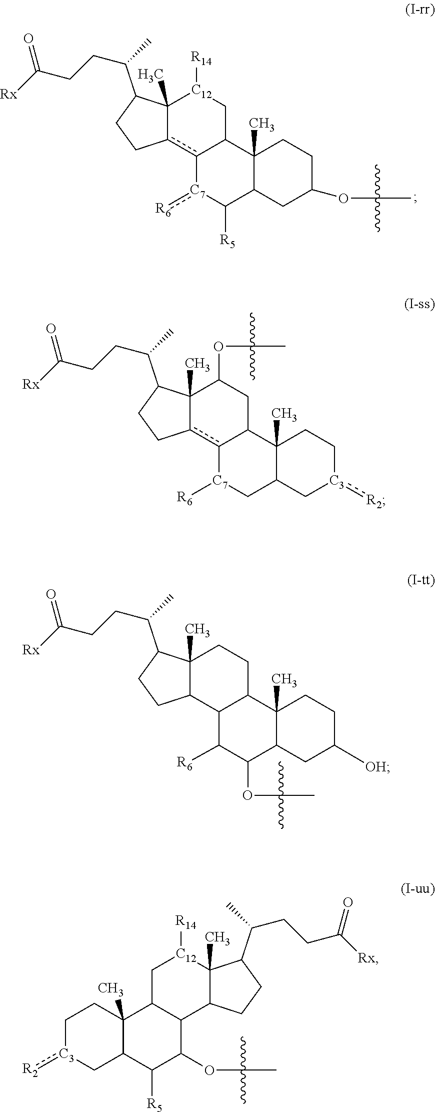

where the bond between C₃ and R₂, C₇ and R₆, and C₁₂ and R₁₄ is a single or a double bond; Rx represents OH, —NHCH₂C(═O)OH, or —NHCH₂CH₂SO₂OH; R₂ represents OH or ═O; R₅ represents H or OH; R₆ represents H, ═O, or OH; R₁₄ represents H, ═O, or OH;

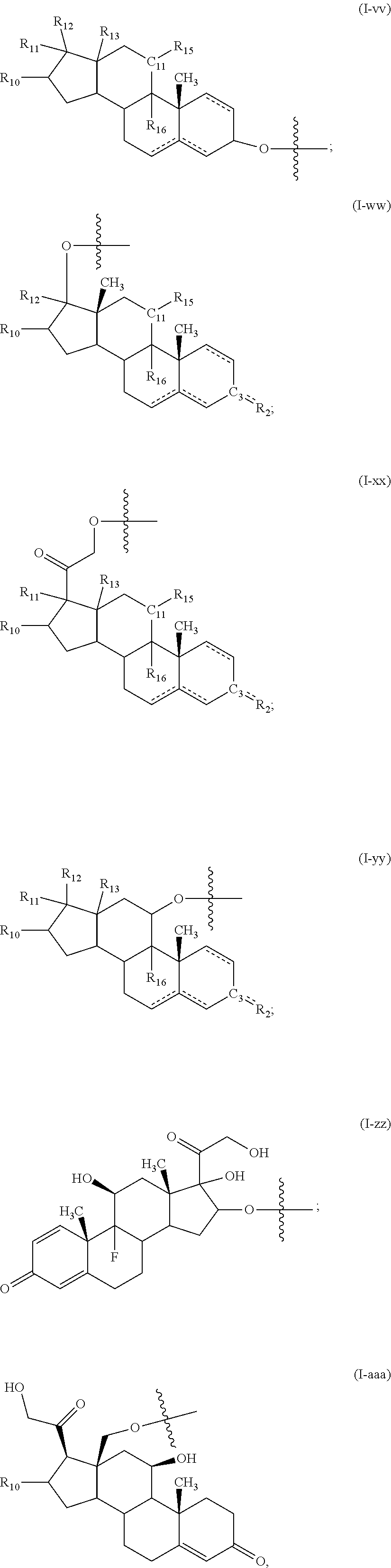

where the bond between C₃ and R₂, and C₁₁ and R₁₅ is a single or a double bond; R₂ represents OH or ═O; R₁₀ represents H or OH; R₁₁ represents H, OH, —C(═O)CH₂OH, —C(═O)OH, —C(═O)CH₂OH, or —C(═O)CH₃; R₁₂ represents H, OH, —C(═O)CH₂OH, —C(═O)OH, —C(═O)CH₂OH, or —C(═O)CH₃; R₁₃ represents —CH₂OH or —CH₃; R₁₅ represents H, OH, or ═O; R₁₆ represents H or F;

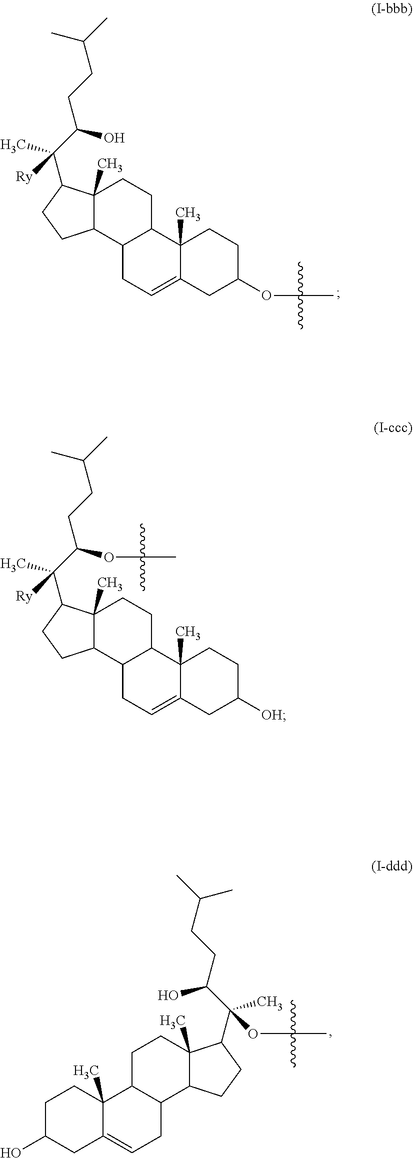

where Ry represents H or OH;

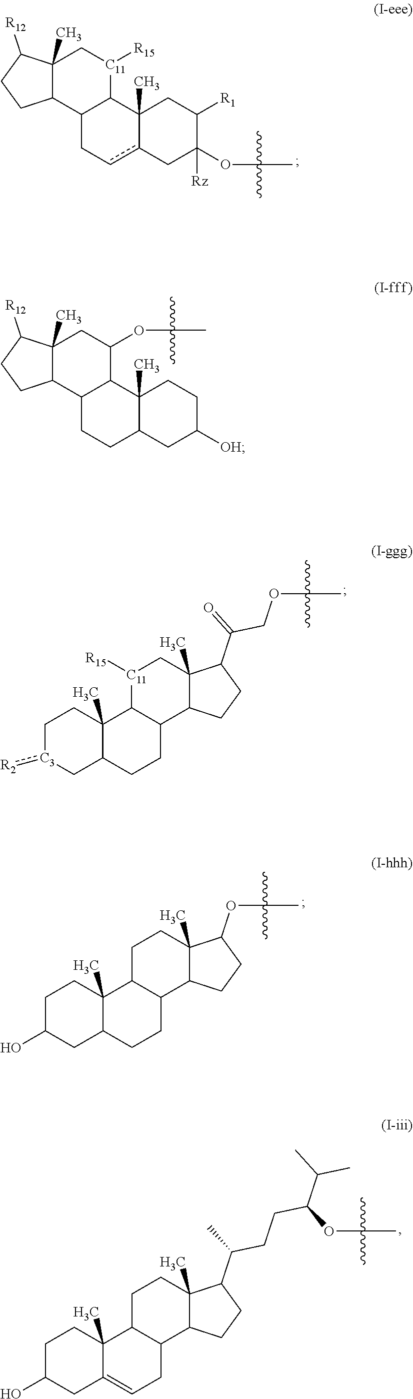

where the bond between C₃ and R₂, and CH and R₁₅ is a single or a double bond; Rz represents H or —CH₃; R₁ represents H or —OCH₂CH₃; R₂ represents OH or ═O; R₁₂ represents —OH, —C(═O)CH₃, —C(═O)CH₂OH, or —CH(CH₃)(CH₂)₂CH(OH)CH(CH₃)₂; R₁₅ represents H, —N(CH₃)₂, or ═O;

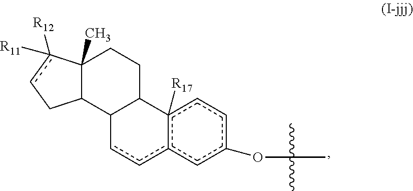

where the bond between C₃ and R₂ is a single or a double bond; R₂ represents OH or ═O; R₁₁ represents H, —C(═O)CH₃, —OC(═O)(CH₂)₄CH₃, or is absent; R₁₂ represents H, —C(═O)CH₃, —OC(═O)(CH₂)₄CH₃, or is absent; R₁₇ represents CH₃ or is absent;

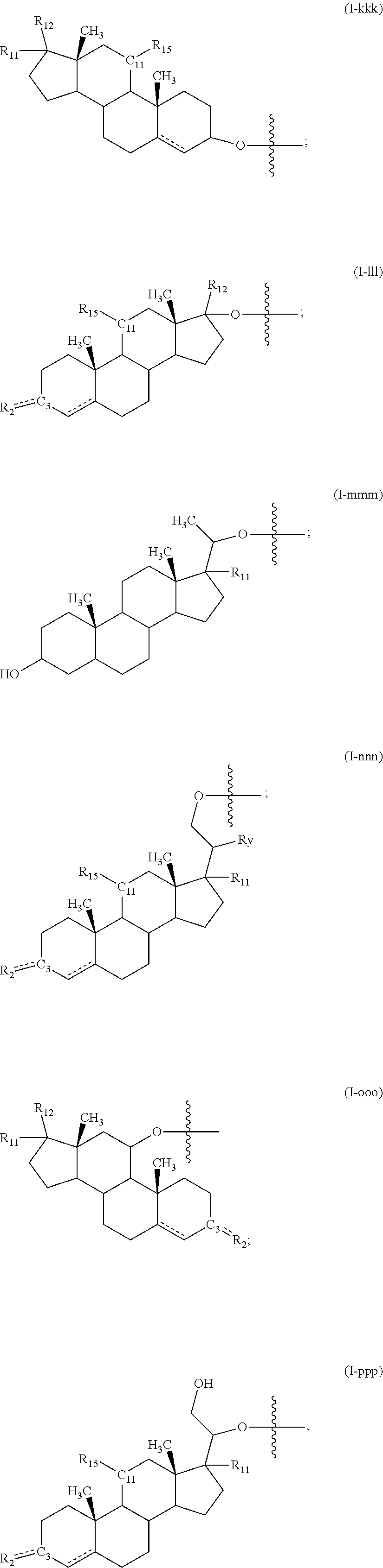

where the bond between C₃ and R₂, and C_{H} and R₁₅ is a single or a double bond; Ry represents OH or ═O; R₂ represents OH or ═O; R₁₁ represents H, OH, —CH(OH)CH₃, —C(═O)CH₂OH, —C(═O)CH₃, or —CH(OH)CH₂OH; R₁₂ represents H, OH, —CH(OH)CH₃, —C(═O)CH₂OH, —C(═O)CH₃, or —CH(OH)CH₂OH; R₁₅ represents H, ═O, or OH;

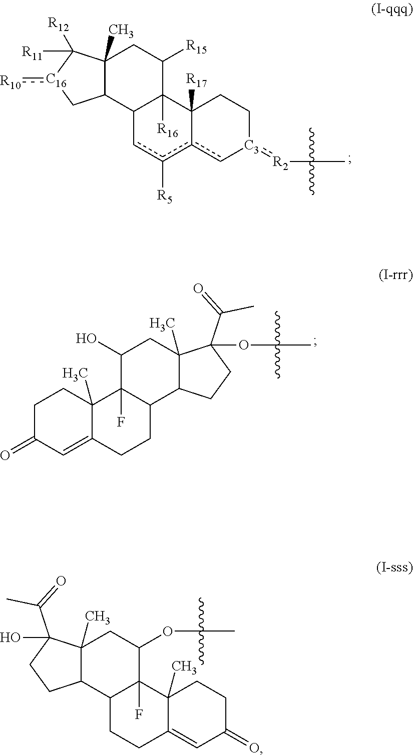

where the bond between C₃ and R₂, and C₁₆ and R₁₀ is a single or a double bond; R₂ represents OH or ═O; R₅ represents H, Cl, or —CH₃; R₁₀ represents H or ═CH₂; R₁₁ represents H, OH, —CH₃, —C(═O)CH₃, —C(═O)CH₂OC(═O)CH₃, or —OC(═O)CH₃; R₁₂ represents H, OH, —CH₃, —C(═O)CH₃, —C(═O)CH₂OC(═O)CH₃, or —OC(═O)CH₃; R₁₅ represents H or OH; R₁₆ represents F or H; R₁₇ represents H or —CH₃; or

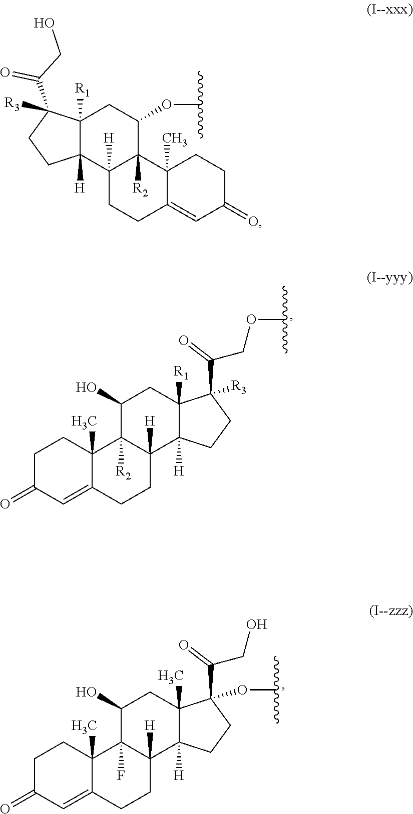

wherein R₁ is C(O)H or CH₃; R₂ represents H or F; R₃ represents H or OH.

In some embodiments, the compound is described by the formula (A-VII):

D1-C(O)-L-C(O)-D2 (A-VII),

or a pharmaceutically acceptable salt thereof, wherein each of D1-C(O) and D2-C(O) is, independently, a radical formed from a steroid; L is —O—C(O)—O—(R^{A})—O—C(O)—O—; and R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. Each of D1-C(O) and D2-C(O) can, independently, be formed, for example, from fusidic acid, cholic acid, chenodeoxycholic acid, ursodeoxycholic acid, or obeticholic acid. In the drug dimers of formula (A-VII), D1-C(O)— and D2-C(O)— can further be described, for example, by formulas (I-hh), (I-ii), (I-ttt), (I-uuu), and (I-vvv) below.

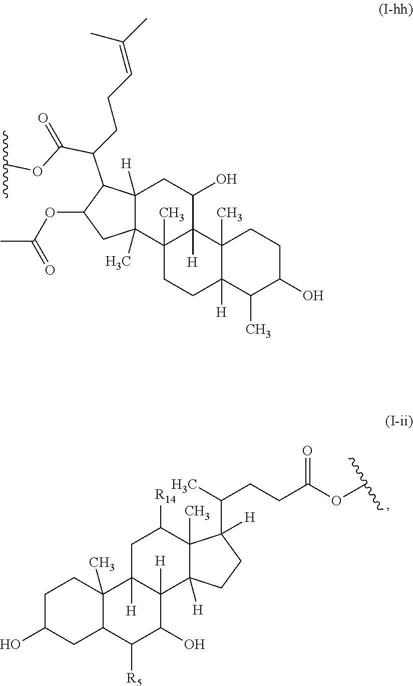

where R₅ represents H or C₁₋₆ alkyl, R₁₄ represents H or OH;

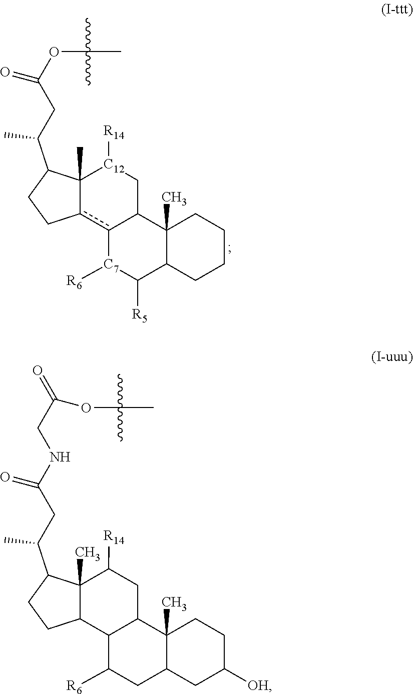

where the bond between C₃ and R₂, C₇ and R₆, and C₁₂ and R₁₄ is a single or a double bond; R₂ represents OH or ═O; R₅ represents H or OH; R₆ represents H, ═O, or OH; R₁₄ represents H, ═O, or OH;

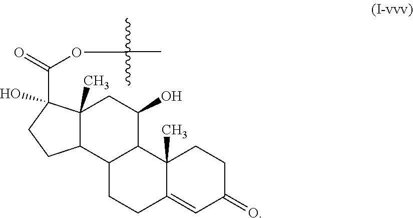 [SMILES: *OC(=O)[C@]1(O)CCC2C3CCC4=CC(=O)CC[C@@]4(C)C3[C@H](O)C[C@]21C]

In some embodiments, drug dimers provided herein include homodimers and heterodimers. In some embodiments, the drug dimers comprise a steroid, including, for example, anabolic steroids, androgenic steroids, progestin steroids, estrogen steroids, cancer treatment steroids, antibiotic steroids, glucocorticoid steroids, benign steroids, corticosteroids, anti-angiogenic steroids, intraocular pressure (IOP) lowering steroids, cholic acid-related bile acid steroids, steroid metabolites, cholesterol-derivatives, mineralocorticoid steroids, neurosteroids, pheromones, progestins, or other steroids. Examples of anabolic steroids include, but are not limited to, androisoxazole, androstenediol, bolandiol, bolasterone, clostebol, ethylestrenol, formyldienolone, 4-hydroxy-19-nortestosterone, methandriol, methenolone, methyltrienolone, nandrolone, norbolethone, oxymesterone, stenbolone, and trenbolone. Androgenic steroids are, for example, boldenone, fluoxymesterone, mestanolone, mesterolone, methandrostenolone, 17-methyltestosterone, 17-α-methyltestosterone 3-cyclopentyl enol ether, norethandrolone, normethandrone, oxandrolone, oxymesterone, oxymetholone, prasterone, stanlolone, stanozolol, testosterone, testosterone 17-chloral hemiacetal, testosterone proprionate, testosterone enanthate tiomesterone dehydroepiandrosterone (DHEA), androstenedione, androstenediol, androsterone, dihydrotestosterone (DHT), androstanolone, and derivatives thereof. Exemplary progestin steroids are norethisterone, norethisterone acetate, gestodene, levonorgestrel, allylestrenol, anagestone, desogestrel, dimethisterone, dydrogesterone, ethisterone, ethynodiol, ethynodiol diacetate, etonogestrel, gestodene, ethinylestradiol, haloprogesterone, 17-hydroxy-16-methylene-progesterone, 17 alpha-hydroxyprogesterone, lynestrenol, medroxyprogesterone, melengestrol, norethindrone, norethynodrel, norgesterone, gestonorone, norethisterone, norgestimate, norgestrel, levonorgestrel, norgestrienone, norvinisterone, pentagestrone, MENT (7-methyl-19-testosterone); norelgestromin, and trimigestone drospirenone, tibolone, megestrol, and derivatives thereof. Examples of estrogen steroid are estrogen, eguilenin, equilin, 17β-estradiol, estradiol benzoate, estriol, ethinyl estradiol, mestranol, moxestrol, mytatrienediol, quinestradiol, and quinestrol. Steroids used in cancer treatment are, for example, abiraterone, cyproterone acetate, dutasteride, enzalutamide, finasteride, and galeterone. Exemplary antibiotic steroid is fusidic acid. Glucocorticoids include, for example, medrysone, alclometasone, alclometasone dipropionate, amcinonide, beclometasone, beclomethasone dipropionate, betamethasone, betamethasone benzoate, betamethasone valerate, budesonide, ciclesonide, clobetasol, clobetasol butyrate, clobetasol propionate, clobetasone, clocortolone, loprednol, cortisol, cortisone, cortivazol, deflazacort, desonide, desoximetasone, desoxycortone, desoxymethasone, dexamethasone, diflorasone, diflorasone diacetate, diflucortolone, diflucortolone valerate, difluorocortolone, difluprednate, fluclorolone, fluclorolone acetonide, fludroxycortide, flumetasone, flumethasone, flumethasone pivalate, flunisolide, flunisolide, fluocinolone, fluocinolone acetonide, fluocinonide, fluocortin, fluocoritin butyl, fluocortolone, fluorocortisone, fluorometholone, fluperolone, fluprednidene, fluprednidene acetate, fluprednisolone, fluticasone, fluticasone propionate, formocortal, halcinonide, halometasone, hydrocortisone, hydrocortisone acetate, hydrocortisone aceponate, hydrocortisone buteprate, hydrocortisone butyrate, loteprednol, meprednisone, 6α-methylprednisolone, methylprednisolone, methylprednisolone acetate, methylprednisolone aceponate, mometasone, mometasone furoate, mometasone furoate monohydrate, paramethasone, prednicarbate, prednisolone, prednisone, prednylidene, rimexolone, tixocortol, triamcinolone, triamcinolone acetonide, and ulobetasol. Exemplary benign steroids are cholesterol, 11-deoxycortisol, 11-deoxycorticosterone, pregnenolone, cholic acid, chenodeoxycholic acid, ursodeoxycholic acid, obeticholic acid, tetrahydrocortisone, tetra hydrodeoxycortisol, tetra hydrocorticosterone, 5α-di hydrocorticosterone, and 5α-dihydropregesterone. Exemplary anti-angiogenic steroids or intraocular pressure (IOP) lowering steroids are anecortave acetate, anecortave, 11-epicortisol, 17α-hydroxyprogesterone, tetrahydrocortexolone, and tetrahydrocortisol. Exemplary cholic acid-related bile acid steroids are deoxycholic acid, apocholic acid, dehydrocholic acid, glycochenodeoxycholic acid, glycocholic acid, glycodeoxycholic acid, hyodeoxycholic acid, lithocholic acid, α-muricholic acid, β-muricholic acid, γ-muricholic acid, ω-muricholic acid, taurochenodeoxycholic acid, taurocholic acid, taurodeoxycholic acid, taurolithocholic acid, and tauroursodeoxycholic acid. Exemplary mineralocorticoid steroids are fludrocortisone and aldocortisone. Exemplary neurosteroids are alphaxalone, alphadolone, hydroxydione, minaxolone, tetrahydrodeoxycorticosterone, allopregnanolone, pregnanolone, ganoxolone, 3α-androstanediol, epipregnanolone, isopregnanolone, and 24(S)-hydroxycholesterol. Exemplary other steroids are flugestone, prebediolone, chlormadinone acetate, medrogestone, and segesterone acetate. Exemplary pheromones are androstadienol, androstadienone, androstenol, androstenone, estratetraenol, 5-dehydroprogesterone, 6-dehydro-retroprogesterone, allopregnanolone, and hydroxyprogesterone caproate. Exemplary steroid metabolites are tetrahydrotriamcinolone, cortienic acid, 11-dehydrocorticosterone, 11β-hydroxypregnenolone, ketoprogesterone, 17-hydroxypregnenolone, 17,21-dihydroxypregnenolone, 18-hydroxycorticosterone, deoxycortisone, 21-hydroxypregnenolone, and progesterone. Exemplary progestins are allopregnone-3α,20α-diol, allopregnone-3β,20β-diol, allopregnane-3β,21-diol-11,20-dione, allopregnane-3β,17α-diol-20-one, 3,20-allopregnanedione,3β,11β,17α, 20β,21-pentol, allopregnane-3β,17α, 20β,21-tetrol, allopregnane-3α,11β,17α, 21-tetrol-20-one, allopregnane-3β,11β,17α, 21-tetrol-20-one, allopregnane-3β,17α,20β-triol, allopregnane-3β,17α, 21-triol-11,20-dione, allopregnane-3β,11β,21-triol-20-one, allopregnane-3β,17α, 21-triol-20-one, allopregnane-3α-ol-20-one, allopregnane-3β-ol-20-one, pregnanediol, 3,20-pregnanedione, 4-pregnene-20,21-diol-3,11-dione, 4-pregnene-11β,17α,20β,21-tetrol-3-one, 4-pregnene-17α, 20β,21-triol-3,11-dione, 4-pregnene-17α,20β,21-triol-3-one, and pregnenolone.

The drug dimers useful in making the articles provided herein can have any of formulas (A-I)-(LXXVIII), described herein.

### Steroid Homodimers

Provided in some embodiments herein are homodimers (e.g., steroid material formed from homodimers) of the formula (I):

D1-L-D2 (A-VIII)

or a pharmaceutically acceptable salt thereof, wherein D1 and D2 are radicals formed from the same steroid. L can be covalently linked to D1 and to D2 via one or more ester, carbonate, carbonate ester, or anhydride linkages. Ester, carbonate, carbonate ester, or anhydride linkages formed from a functional group on D1 and D2 can be selected from, e.g., hydroxyl or carboxy. For example, L can include the radical —C(O)—(R^{A})—C(O)—, —C(O)—OC(O)—(R^{A})—C(O)O—C(O)—, or —O—(R^{A})—O—, where R^{A} is a radical of a polyol and includes at least one free hydroxyl group or R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, —(CH₂CH₂O)_{q}CH₂CH₂—, —(CH₂CH₂CH₂CH₂O)ᵣCH₂CH₂CH₂CH₂—, or —(CH₂CH(CH₃)O)ₛCH₂CH(CH₃)—, and q, r, and s are integers from 1 to 10 (e.g., 1 to 10, 1 to 5, or 5 to 10). The homodimer can be further described by one of formulas (II)-(LXXVIII), below.

In some embodiments, the steroid is an anabolic steroid and the drug dimer is further described by the formula (II):

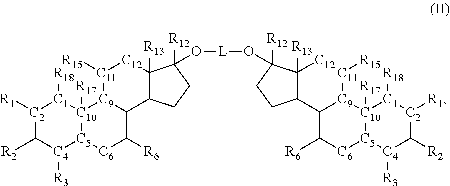

wherein the bond between C₁ and C₂, C₄ and C₅, C₅ and C₆, C₉ and C₁₀, and C_{H} and C₁₂ is a single or a double bond; R₁ represents H, CH₃, or HC(O); R₂ represents ═O, OH, or H; or R₁ and R₂ taken together with carbons to which they are attached form an isoxazole; R₃ represents H, a halogen atom, or OH; R₆ represents H or CH₃; R₁₂ represents H, CH₃, or CH₃CH₂; R₁₃ represents CH₃ or CH₃CH₂; R₁₅ represents H or OH; R₁₇ represents H or CH₃; R₁₈ represents H or CH₃; L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₚCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (II) can be formed from an anabolic steroid selected from the group consisting of androisoxazole, androstenediol, bolandiol, bolasterone, clostebol, ethylestrenol, formyldienolone, 4-hydroxy-19-nortestosterone, methandriol, methenolone, methyltrienolone, nandrolone, norbolethone, oxymesterone, stenbolone, and trenbolone.

In certain embodiments, the steroid is an anabolic steroid and the drug dimer is further described by the formula (III):

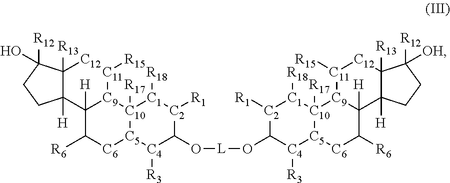

wherein the bond between C₁ and C₂, C₄ and C₅, C₅ and C₆, C₉ and C₁₀, and C₁₁ and C₁₂ is a single or a double bond; R₁ represents H, CH₃, or HC(O); R₃ represents H, a halogen atom, or OH; R₆ represents H or CH₃; R₁₂ represents H, CH₃, or CH₃CH₂; Rn represents CH₃ or CH₃CH₂; R₁₅ represents H or OH; R₁₇ represents H or CH₃; R₁₈ represents H or CH₃; L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₚCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (III) can be formed from an anabolic steroid selected from the group consisting of androstenediol, bolandiol, bolasterone, clostebol, formyldienolone, 4-hydroxy-19-nortestosterone, methandriol, methenolone, methyltrienolone, nandrolone, norbolethone, oxymesterone, stenbolone, and trenbolone.

In some embodiments, the steroid is an anabolic steroid and the drug dimer is further described by the formula (IV):

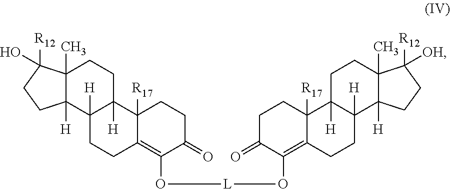

wherein R₁₂ represents H or CH₃; R₁₇ represents H or CH₃; L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (IV) can be formed from an anabolic steroid selected from 4-hydroxy-19-nortestosterone or oxymesterone.

In certain embodiments, the steroid is an androgenic steroid and the drug dimer is further described by the formula (V):

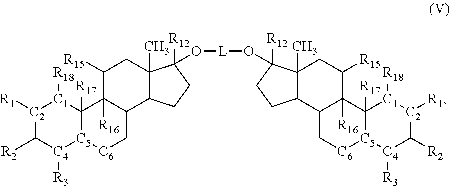

wherein the bond between C₁ and C₂, C₄ and C₅, and C₅ and C₆ is a single or a double bond; C₂ is O, C or CH₂; R₁ represents H, —CHOH, or is absent; R₂ represents ═O or OH; or R₁ and R₂ taken together with carbons to which they are attached form a pyrazole; R₃ represents H or OH; R₁₂ represents H, CH₃, optionally substituted alkynylene, C₁₋₆ alkoxy, or CH₃CH₂; R₁₅ represents H or OH; R₁₆ represents H or a halogen atom; R₁₇ represents H or CH₃; R₁₈ represents H or CH₃; L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from:
—O(CH₂CH₂O)ₙCH₂CH₂O—, —O(CH₂CH₂CH₂CH₂O)ₘCH₂CH₂CH₂CH₂O—, or
—O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (V) can be formed from an androgenic steroid selected from the group consisting of boldenone, fluoxymesterone, mestanolone, mesterolone, methandrostenolone, 17-methyltestosterone, 17-α-methyltestosterone 3-cyclopentyl enol ether, norethandrolone, normethandrone, oxandrolone, oxymesterone, oxymetholone, prasterone, stanlolone, stanozolol, testosterone, testosterone enanthate tiomesterone dehydroepiandrosterone (DHEA), androstenedione, androstenediol, androsterone, and dihydrotestosterone (DHT).

In some embodiments, the steroid is an androgenic steroid and the drug dimer is further described by the formula (VI):

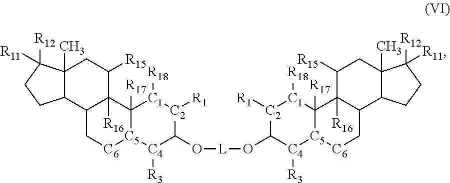

wherein the bond between C₁ and C₂, C₄ and C₅, and C₅ and C₆ is a single or a double bond; C₂ is O, C or CH₂; R₁ represents H, —CHOH, or is absent; R₃ represents H or OH; R₁₁ represents H, OH, CH₃, optionally substituted alkynylene, CH₃CH₂, ═O, —OC(O)CH₂CH₃, or is absent; R₁₂ represents H, OH, CH₃, optionally substituted alkynylene, CH₃CH₂, ═O, —OC(O)CH₂CH₃, or is absent; R₁₅ represents H or OH; R₁₆ represents H or a halogen atom; R₁₇ represents H or CH₃; R₁₈ represents H or CH₃; L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—; or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (VI) can be formed from an androgenic steroid selected from the group consisting of boldenone, fluoxymesterone, mestanolone, mesterolone, methandrostenolone, 17-methyltestosterone, norethandrolone, normethandrone, oxandrolone, oxymesterone, oxymetholone, prasterone, stanlolone, testosterone, testosterone proprionate, testosterone enanthate tiomesterone dehydroepiandrosterone (DHEA), androstenedione, androstenediol, androsterone, and dihydrotestosterone (DHT).

In certain embodiments, the steroid is an androgenic steroid and the drug dimer is further described by the formula (VII):

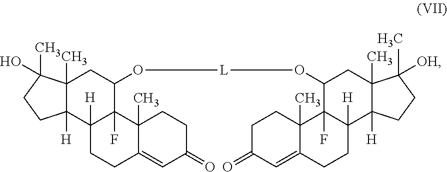

wherein L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (VII) can be formed from the androgenic steroid fluoxymesterone.

In some embodiments, the steroid is an androgenic steroid and the drug dimer is further described by the formula (VIII):

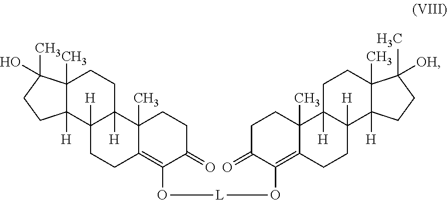

wherein L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (VIII) can be formed from the androgenic steroid oxymesterone.

In some embodiments, the steroid is an androgenic steroid and the drug dimer is further described by the formula (IX):

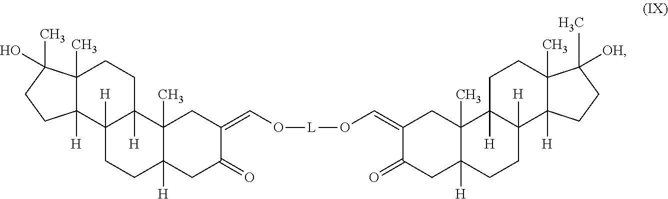

wherein L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (IX) can be formed from the androgenic steroid oxymetholone.

In some embodiments, the steroid is a progestin steroid and the drug dimer is further described by the formula (X):

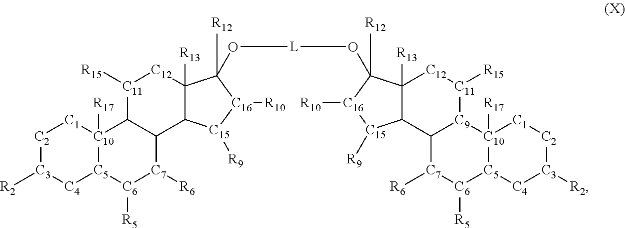

wherein the bond between C₁ and C₁₀, C₂ and C₃, C₃ and C₄, C₄ and C₅, C₅ and C₆, C₆ and C₇, C₅ and Cm, C₉ and C₁₀, CH and C₁₂, C₁₅ and C₁₆ is a single or a double bond; R₂ represents H, ═O, OH, —NOH, or C₁₋₆alkoxy; R₅ represents H, CH₃, or a halogen atom; R₆ represents H or CH₃; or R₅ and R₆ taken together with carbons to which they are attached form a cyclopropane; R₉ is H; R₁₀ is H or ═CH₂; or R₉ and R₁₀ taken together with carbons to which they are attached form a cyclopropane; R₁₂ represents H, optionally substituted alkynylene, —CH₂CH═CH₂, CH₃, —C(O)CH₃, or —CH═CH₂; R₁₃ represents CH₃ or CH₂CH₃; R₁₅ represents H or ═CH₂; R₁₇ represents H, CH₃, or is absent; L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from:
—O(CH₂CH₂O)ₙCH₂CH₂O—, —O(CH₂CH₂CH₂CH₂O)ₘCH₂CH₂CH₂CH₂O—, or
—O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (X) can be formed from a progestin steroid selected from the group consisting of norethisterone, gestodene, levonorgestrel, allylestrenol, anagestone, desogestrel, dimethisterone, dydrogesterone, ethisterone, ethynodiol, etonogestrel, gestodene, ethinylestradiol, 17-hydroxy-16-methylene-progesterone, 17 alpha-hydroxyprogesterone, lynestrenol, medroxyprogesterone, melengestrol, norethindrone, norethynodrel, norgesterone, gestonorone, norethisterone, norgestrel, levonorgestrel, norgestrienone, pentagestrone, 7-methyl-19-testosterone (MENT), norelgestromin, tibolone, and megestrol.

In certain embodiments, the steroid is a progestin steroid and the drug dimer is further described by the formula (XI):

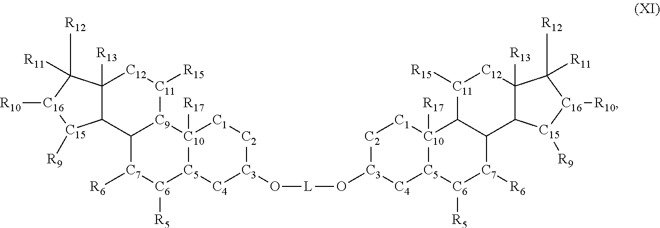

wherein the bond between C₁ and C₁₀, C₂ and C₃, C₄ and C₅, C₆ and C₇, C₅ and C₁₀, C₉ and C₁₀, CH and C₁₂, C₁₅ and C₁₆ is a single or a double bond; R₅ represents H, CH₃, or a halogen atom; R₆ represents H or CH₃; or R₅ and R₆ taken together with carbons to which they are attached form a cyclopropane; R₉ is H; R₁₀ is H or ═CH₂; or R₉ and R₁₀ taken together with carbons to which they are attached form a cyclopropane; R¹¹ represents H, OH, optionally substituted alkynylene, —C(O)CH₃, —CH₂CH═CH₂, a halogen atom, —CH═CH₂, —OC(O)CH₃, CH₃, —C(O)C(OH)CH₃; R₁₂ represents H, OH, optionally substituted alkynylene, —C(O)CH₃, —CH₂CH═CH₂, a halogen atom, —CH═CH₂, —OC(O)CH₃, CH₃, —C(O)C(OH)CH₃; or Rn and R₁₂ together with carbon to which they are attached form a lactone; R₁₃ represents CH₃ or CH₂CH₃; R₁₅ represents H or ═CH₂; R₁₇ represents H, CH₃, or is absent; L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from:
—O(CH₂CH₂O)ₙCH₂CH₂O—, —O(CH₂CH₂CH₂CH₂O)ₘCH₂CH₂CH₂CH₂O—, or
—O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (XI) can be formed from a progestin steroid selected from the group consisting of norethisterone, norethisterone acetate, gestodene, levonorgestrel, dimethisterone, dydrogesterone, ethisterone, ethynodiol, etonogestrel, gestodene, ethinylestradiol, haloprogesterone, 17-hydroxy-16-methylene-progesterone, 17 alpha-hydroxyprogesterone, medroxyprogesterone, melengestrol, norethindrone, norethynodrel, norgesterone, gestonorone, norethisterone, norgestimate, norgestrel, levonorgestrel, norgestrienone, 7-methyl-19-testosterone (MENT), norelgestromin, trimigestone, drospirenone, tibolone, and megestrol.

In some embodiments, the steroid is a progestin steroid and the drug dimer is further described by the formula (XII):

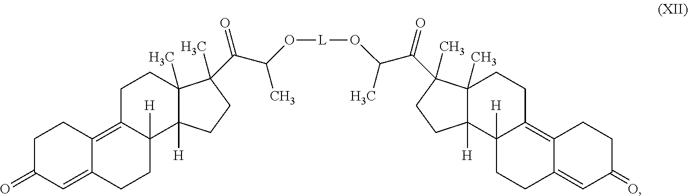

wherein L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (XII) can be formed from the progestin steroid trimigestone.

In some embodiments, the steroid is an estrogen steroid and the drug dimer is further described by the formula (XIII):

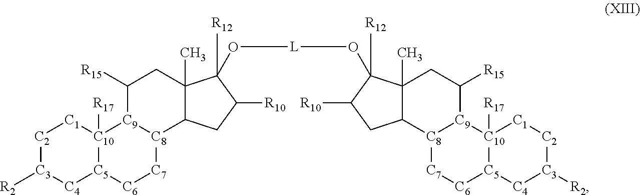

wherein the bond between C₁ and C₂, C₁ and C₁₀, C₂ and C₃, C₃ and C₄, C₄ and C₅, C₆ and C₇, C₅ and C₁₉, C₇ and C₈, and C₈ and C₉ is a single or a double bond; R₂ represents OH, —OC(O)Ph, or C₁-6 alkoxy; R₁₀ represents H or OH; R₁₂ represents H, optionally substituted alkynylene; R₁₅ represents H or C₁₋₆ alkoxy; L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₉ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (XIII) can be formed from an estrogen steroid selected from the group consisting of estrogen, eguilenin, equilin, 17β-estradiol, estradiol benzoate, estriol, ethinyl estradiol, mestranol, moxestrol, mytatrienediol, quinestradiol, and quinestrol.

In some embodiments, the steroid is an estrogen steroid and the drug dimer is further described by the formula (XIV):

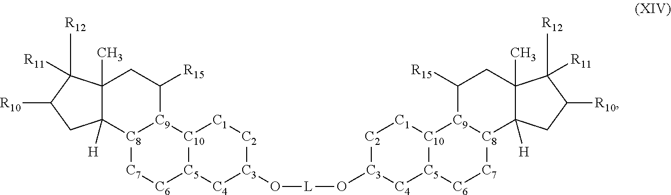

wherein the bond between C₁ and C₂, C₁ and C₁₀, C₂ and C₃, C₃ and C₄, C₄ and C₅, C₆ and C₇, C₅ and C₁₀, C₇ and C₈, and C₈ and C₉ is a single or a double bond; R₁₀ represents H or OH; Rn represents H, OH, optionally substituted alkynylene, ═O, or is absent; R₁₂ represents H, OH, optionally substituted alkynylene, ═O, or is absent; R₁₅ represents H or C₁₋₆ alkoxy; L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (XIV) can be formed from an estrogen steroid selected from the group consisting of estrogen, eguilenin, equilin, 17β-estradiol, estriol, ethinyl estradiol, and moxestrol.

In some embodiments, the steroid is an estrogen steroid and the drug dimer is further described by the formula (XV):

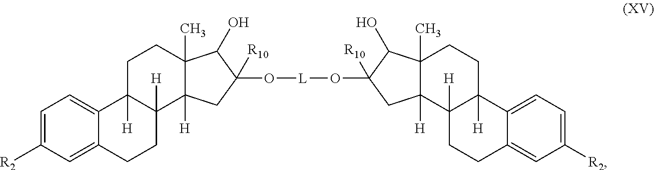

wherein R₂ represents OH or C₁₋₆ alkoxy; R₁₀ represents H or CH₃; L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from:
—O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (XV) can be formed from an estrogen steroid selected from the group consisting of estriol, mytatrienediol, and quinestradiol.

In some embodiments, the steroid is a cancer treatment steroid and the drug dimer is further described by the formula (XVI):

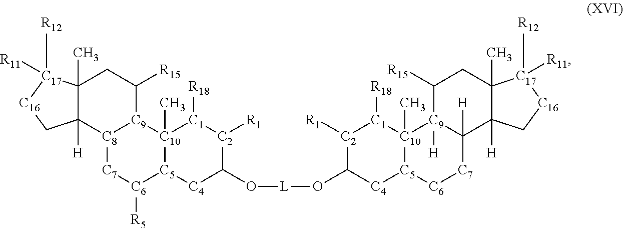

wherein the bond between C₁ and C₂, C₄ and C₅, C₅ and C₆, C₆ and C₇, and C₁₆ and C₁₇ is a single or a double bond; C₄ is NH, CH, or CH₂; R₁ represents H; R₅ represents H or a halogen atom; R₁₁ represents H, optionally substituted heteroaryl, —C(O)C₁₋₆ alkyl, —C(O)OC₁₋₆ alkyl, or —C(O)NHR, wherein R is optionally substituted alkyl or aryl; R₁₂ represents H, optionally substituted heteroaryl, —C(O)C₁₋₆alkyl, —C(O)OC₁₋₆alkyl, or —C(O)NHR, wherein R is optionally substituted alkyl or aryl; R₁₈ represents H; or R₁ and R₁₈ taken together with carbons to which they are attached form a cyclopropane; L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from:
—O (CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (XVI) can be formed from a cancer treatment steroid selected from the group consisting of abiraterone, cyproterone acetate, dutasteride, finasteride, and galeterone.

In some embodiments, the steroid is an antibiotic steroid and the drug dimer is further described by the formula (XVII):

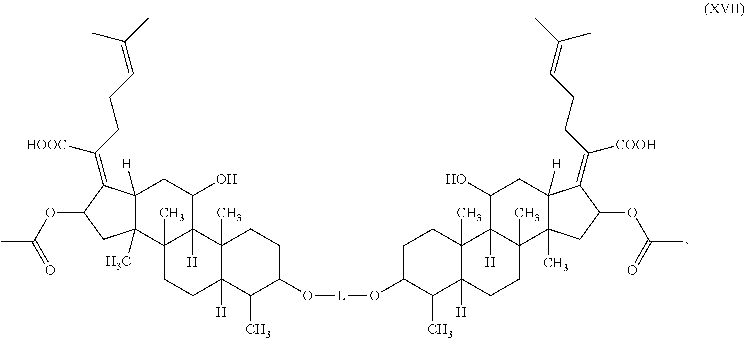

wherein L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or
O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (XVII) can be formed from the steroid antibiotic fusidic acid.

In some embodiments, the steroid is an antibiotic steroid and the drug dimer is further described by the formula (XVIII):

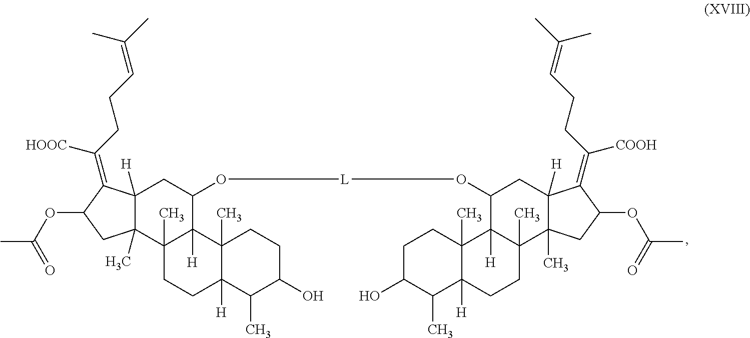

wherein L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or
O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (XVIII) can be formed from the steroid antibiotic fusidic acid.

In some embodiments, the steroid is a benign steroid and the drug dimer is further described by the formula (XIX):

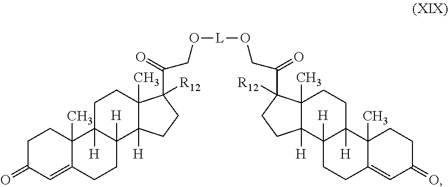

wherein R₁₂ is H or OH; L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (XIX) can be formed from a benign steroid selected from 11-deoxycortisol and 11-deoxycorticosterone.

In some embodiments, the steroid is a benign steroid and the drug dimer is further described by the formula (XX):

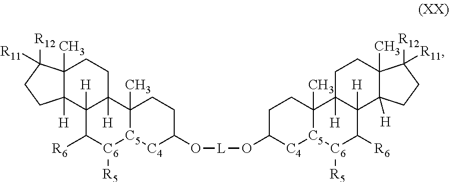

wherein the bond between C₄ and C₅, and C₅ and C₆ is a single or a double bond; R₅ represents H or C₁₋₆ alkyl; R₆ represents H or OH; Rn represents H, OH, —C(O)C₁₋₆ alkyl, —C(O)CH₂OH, or —CH(CH₃)CH₂CH₂C(O)OH; R₁₂ represents H, OH, —C(O)C₁₋₆ alkyl, —C(O)CH₂OH, or —CH(CH₃)CH₂CH₂C(O)OH; L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (XX) can be formed from a benign steroid selected from the group consisting of cholesterol, 11-deoxycortisol, 11-deoxycorticosterone, pregnenolone, cholic acid, chenodeoxycholic acid, ursodeoxycholic acid, and obeticholic acid.

In some embodiments, the steroid is a benign steroid and the drug dimer is further described by the formula (XXI):

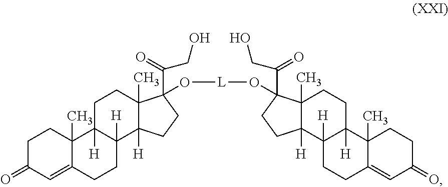

wherein L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms.

The drug dimer of formula (XXI) can be formed from a benign steroid including 11-deoxycortisol.

In some embodiments, the steroid is a benign steroid and the drug dimer is further described by the formula (XXII):

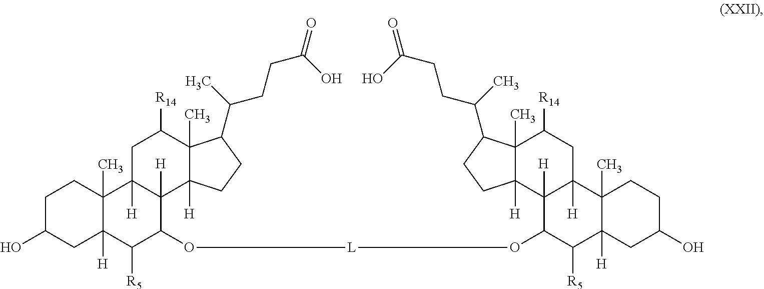

wherein R₅ represents H or CH₂CH₃; R₁₄ represents H or OH; L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (XXII) can be formed from a benign steroid selected from the group consisting of cholic acid, chenodeoxycholic acid, ursodeoxycholic acid, and obeticholic acid.

In some embodiments, the steroid is a benign steroid and the drug dimer is further described by the formula (XXIII):

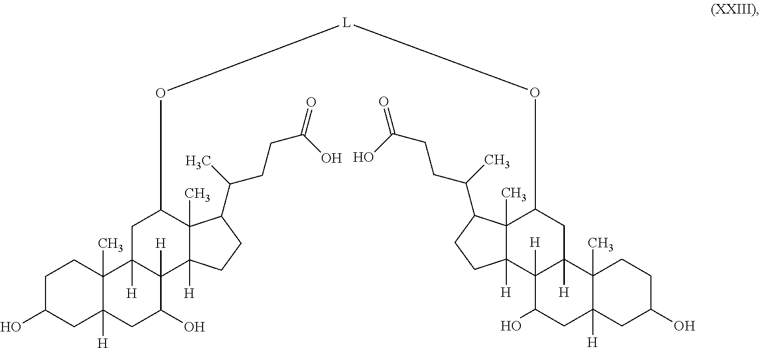

wherein L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (XXIII) can be formed from the benign steroid cholic acid.

In some embodiments, the steroid is a glucocorticoid steroid and the drug dimer is further described by the formula (XXIV):

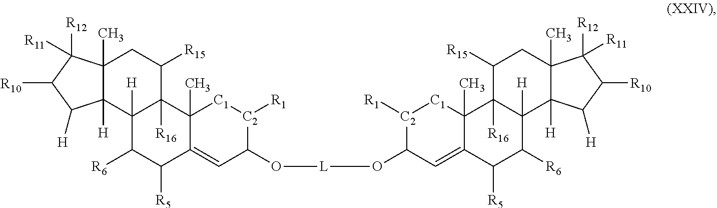

wherein the bond between C₁ and C₂ is a single or a double bond; R₁ represents H or a halogen atom; R₅ represents H, C₁₋₆ alkyl, or a halogen atom; R₆ represents H or a halogen atom; R₁₀ represents H, C₁₋₆ alkyl, OH, or ═CH₂; R₁₁ represents H, OH, C₁₋₆ alkyl, optionally substituted —C(O)C₁₋₆ alkyl, —C(O)CH₂OC(O)C₁₋₆ alkyl, optionally substituted —OC(O)C₁₋₆ alkyl, —OC(O)Ph, —OC(O)heterocyclyl, —CH₂C(O)CH₂OH, —C(O)C(O)OH, —C(O)C(O)OC₁₋₆ alkyl, —C(O)SCH₂F, or —OC(O)OC₁₋₆alkyl; or R₁₀ and R₁₁ taken together with carbons to which they are attached form an optionally substituted cyclic acetal or optionally substituted heterocyclyl; R₁₂ represents H, OH, C₁₋₆ alkyl, optionally substituted —C(O)C₁₋₆ alkyl, —C(O)CH₂OC(O)C₁₋₆ alkyl, optionally substituted —OC(O)C₁₋₆ alkyl, —OC(O)Ph, —OC(O)heterocyclyl, —CH₂C(O)CH₂OH, —C(O)C(O)OH, —C(O)C(O)OC₁₋₆ alkyl, —C(O)SCH₂F, or —OC(O)OC₁₋₆alkyl; or R₁₀ and R₁₂ taken together with carbons to which they are attached form an optionally substituted cyclic acetal or optionally substituted heterocyclyl; R₁₅ represents H, OH, ═O, or a halogen atom; R₁₆ represents H or a halogen atom; L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (XXIV) can be formed from a glucocorticoid steroid selected from the group consisting of medrysone, alclometasone, alclometasone dipropionate, amcinonide, beclometasone, beclomethasone dipropionate, betamethasone, betamethasone benzoate, betamethasone valerate, budesonide, ciclesonide, clobetasol, clobetasol butyrate, clobetasol propionate, clobetasone, clocortolone, cortisol, cortisone, deflazacort, desonide, desoximetasone, desoxycortone, desoxymethasone, dexamethasone, diflorasone, diflorasone diacetate, diflucortolone, diflucortolone valerate, difluorocortolone, difluprednate, fluclorolone, fluclorolone acetonide, fludroxycortide, flumetasone, flumethasone, flumethasone pivalate, flunisolide, flunisolide, fluocinolone, fluocinolone acetonide, fluocinonide, fluocortin, fluocoritin butyl, fluocortolone, fluorocortisone, fluorometholone, fluperolone, fluprednidene, fluprednidene acetate, fluprednisolone, fluticasone, fluticasone propionate, halcinonide, halometasone, hydrocortisone, hydrocortisone acetate, hydrocortisone aceponate, hydrocortisone buteprate, hydrocortisone butyrate, loteprednol, meprednisone, 6α-methylprednisolone, methylprednisolone, methylprednisolone acetate, methylprednisolone aceponate, mometasone, mometasone furoate, mometasone furoate monohydrate, paramethasone, prednicarbate, prednisolone, prednisone, prednylidene, rimexolone, tixocortol, triamcinolone, triamcinolone acetonide, and ulobetasol.

In some embodiments, the steroid is a glucocorticoid steroid and the drug dimer is further described by the formula (XXV):

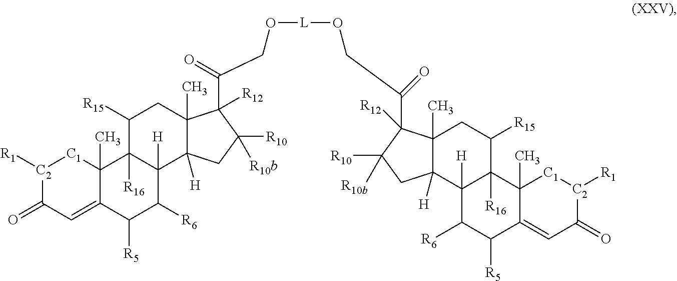

wherein the bond between C₁ and C₂ is a single or a double bond; R₁ represents H or a halogen atom; R₅ represents H, C₁₋₆ alkyl, or a halogen atom; R₆ represents H or a halogen atom; R₁₀ represents H, C₁₋₆ alkyl, OH, or ═CH₂; REM represents H, C₁₋₆ alkyl, OH, ═CH₂, or be absent; R₁₂ represents H, OH, optionally substituted —C(O)C₁₋₆ alkyl, —C(O)CH₂OC(O)C₁₋₆ alkyl, optionally substituted —OC(O)C₁₋₆ alkyl, or —OC(O)Ph; or R₁₀ and R₁₁ taken together with carbons to which they are attached form an optionally substituted cyclic acetal or optionally substituted heterocyclyl; R₁₅ represents H, OH, ═O, or a halogen atom; R₁₆ represents H or a halogen atom; L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—,
—O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (XXV) can be formed from a glucocorticoid steroid selected from the group consisting of alclometasone, beclometasone, betamethasone, betamethasone benzoate, betamethasone valerate, budesonide, cortisol, cortisone, desonide, desoximetasone, desoxycortone, desoxymethasone, dexamethasone, diflorasone, diflucortolone, difluorocortolone, fluclorolone, fluclorolone acetonide, fludroxycortide, flumetasone, flumethasone, flunisolide, flunisolide, fluocinolone, fluocinolone acetonide, fluocortolone, fluorocortisone, fluprednidene, fluprednisolone, halometasone, hydrocortisone, hydrocortisone butyrate, meprednisone, 6α-methylprednisolone, methylprednisolone, paramethasone, prednisolone, prednisone, prednylidene, triamcinolone, and triamcinolone acetonide.

In some embodiments, the steroid is a glucocorticoid steroid and the drug dimer is further described by the formula (XXVI):

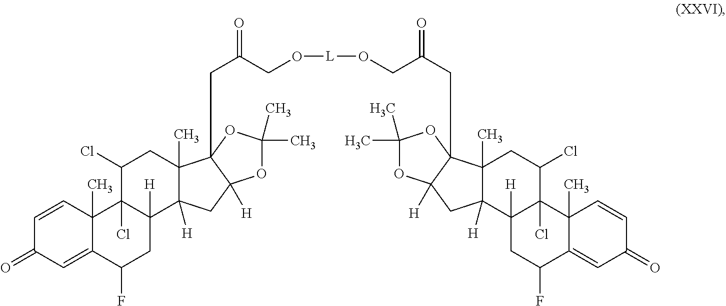

wherein L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (XXVI) can be formed from the glucocorticoid steroid fluclorolone acetonide.

In some embodiments, the steroid is a glucocorticoid steroid and the drug dimer is further described by the formula (XXVII):

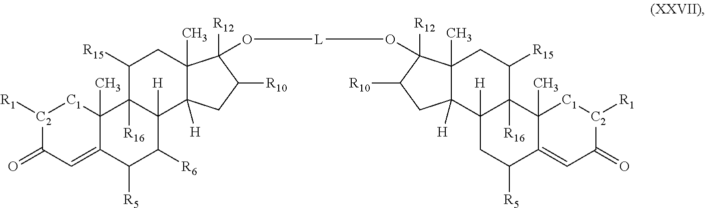

wherein the bond between C₁ and C₂ is a single or a double bond; R₁ represents H or a halogen atom; R₅ represents H, a halogen atom, or CH₃; R₆ represents H, a halogen atom; R₁₀ represents H, OH, CH₃, or ═CH₂; R₁₂ represents optionally substituted —C(O)C₁₋₆alkyl, —C(O)CH₂OC(O)C₁₋₆alkyl, or —C(O)SCH₂F; R₁₅ represents OH or ═O; R₁₆ represents H or a halogen atom; L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})— is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (XXVII) can be formed from a glucocorticoid steroid selected from the group consisting of alclometasone, beclometasone, betamethasone, clobetasol, clobetasone, cortisol, cortisone, dexamethasone, diflorasone, fluclorolone, flumetasone, flumethasone, flumethasone pivalate, fluocinolone, fluorocortisone, fluorometholone, fluperolone, fluprednidene, fluprednidene acetate, fluprednisolone, fluticasone, halometasone, hydrocortisone, hydrocortisone acetate, hydrocortisone butyrate, meprednisone, 6α-methylprednisolone, methylprednisolone, methylprednisolone acetate, mometasone, paramethasone, prednisolone, prednisone, prednylidene, tixocortol, triamcinolone, and ulobetasol.

In some embodiments, the steroid is a glucocorticoid steroid and the drug dimer is further described by the formula (XXVIII):

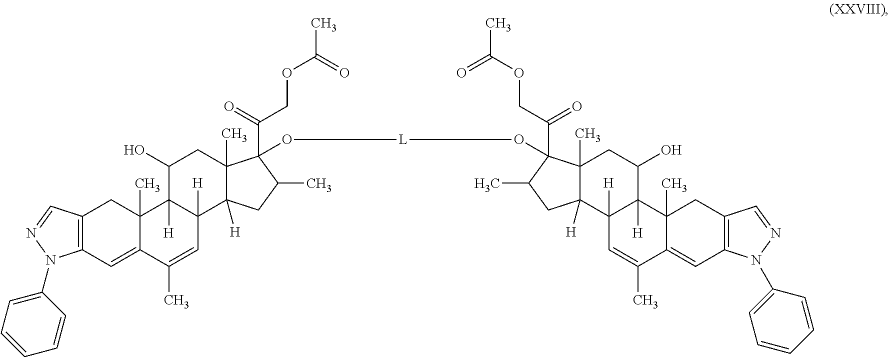

wherein L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (XXVIII) can be formed from the glucocorticoid steroid cortivazol.

In some embodiments, the steroid is a glucocorticoid steroid and the drug dimer is further described by the formula (XXIX):

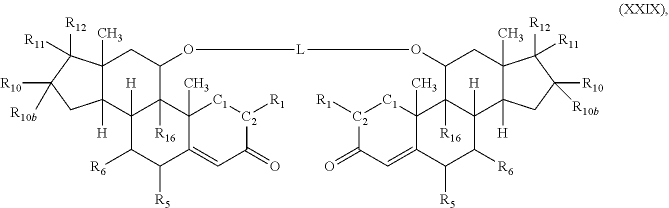

wherein the bond between C₁ and C₂ is a single or a double bond; R₁ represents H or a halogen atom; R₅ represents H, C₁₋₆ alkyl, or a halogen atom; R₆ represents H or a halogen atom; R₁₀ represents H, C₁₋₆ alkyl, OH, or ═CH₂; REM represents H, C₁₋₆ alkyl, OH, or ═CH₂, or is absent; R₁₁ represents H, OH, C₁₋₆ alkyl, optionally substituted —C(O)C₁₋₆ alkyl, —C(O)CH₂OC(O)C₁₋₆ alkyl, optionally substituted —OC(O)C₁₋₆ alkyl, —OC(O)Ph, —OC(O)heterocyclyl, —CH₂C(O)CH₂OH, —C(O)C(O)OH, —C(O)C(O)OC₁₋₆alkyl, —C(O)SCH₂F, or —OC(O)OC₁₋₆alkyl; or R₁₀ and R₁₁ taken together with carbons to which they are attached form an optionally substituted cyclic acetal or optionally substituted heterocyclyl; R₁₂ represents H, OH, C₁₋₆ alkyl, optionally substituted —C(O)C₁₋₆ alkyl, —C(O)CH₂OC(O)C₁₋₆ alkyl, optionally substituted —OC(O)C₁₋₆ alkyl, —OC(O)Ph, —OC(O)heterocyclyl, —CH₂C(O)CH₂OH, —C(O)C(O)OH, —C(O)C(O)OC₁₋₆ alkyl, —C(O)SCH₂F, or —OC(O)OC₁₋₆ alkyl; or R₁₀ and R₁₂ taken together with carbons to which they are attached form an optionally substituted cyclic acetal or optionally substituted heterocyclyl; R₁₆ represents H or a halogen atom; L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (XXIX) can be formed from a glucocorticoid steroid selected from the group consisting of medrysone, alclometasone, alclometasone dipropionate, amcinonide, beclometasone, beclomethasone dipropionate, betamethasone, betamethasone benzoate, betamethasone valerate, budesonide, ciclesonide, clobetasol, clobetasol butyrate, clobetasol propionate, clobetasone, clocortolone, cortisol, cortisone, cortivazol, deflazacort, desonide, desoximetasone, desoxymethasone, dexamethasone, diflorasone, diflorasone diacetate, diflucortolone, diflucortolone valerate, difluorocortolone, difluprednate, fludroxycortide, flumetasone, flumethasone, flumethasone pivalate, flunisolide, flunisolide, fluocinolone, fluocinolone acetonide, fluocinonide, fluocortin, fluocoritin butyl, fluocortolone, fluorocortisone, fluorometholone, fluperolone, fluprednidene, fluprednidene acetate, fluprednisolone, fluticasone, fluticasone propionate, formocortal, halcinonide, halometasone, hydrocortisone, hydrocortisone acetate, hydrocortisone aceponate, hydrocortisone buteprate, hydrocortisone butyrate, loteprednol, meprednisone, 6α-methylprednisolone, methylprednisolone, methylprednisolone acetate, methylprednisolone aceponate, mometasone, mometasone furoate, mometasone furoate monohydrate, paramethasone, prednicarbate, prednisolone, prednisone, prednylidene, rimexolone, tixocortol, triamcinolone, triamcinolone acetonide, and ulobetasol.

In some embodiments, the steroid is a glucocorticoid steroid and the drug dimer is further described by the formula (XXX):

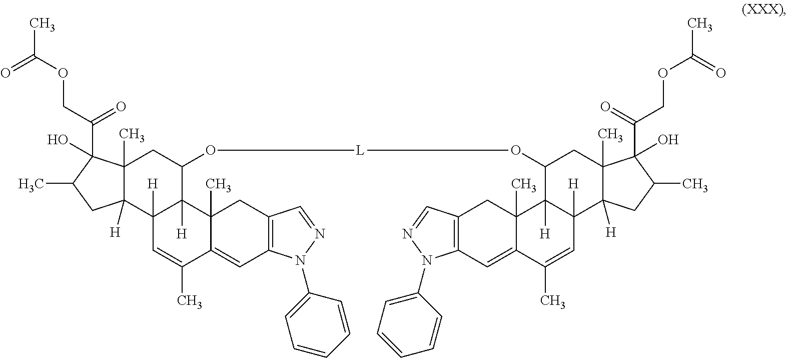

wherein L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (XXX) can be formed from the glucocorticoid steroid cortivazol.

In some embodiments, the steroid is a glucocorticoid steroid and the drug dimer is further described by the formula (XXXI):

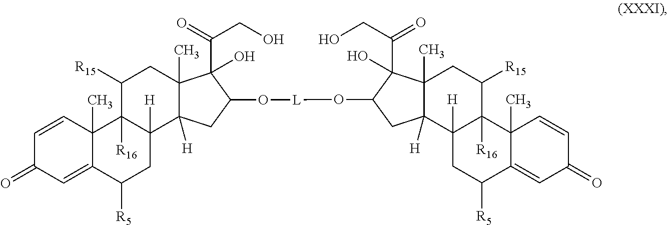

wherein R₅ represents H or a halogen atom; R₁₅ represents a halogen atom or OH; R₁₆ represents H or a halogen atom; L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (XXXI) can be formed from a glucocorticoid steroid selected from the group consisting of fluclorolone, fluocinolone, and triamcinolone.

In some embodiments, the steroid is a glucocorticoid steroid and the drug dimer is further described by the formula (XXXII):

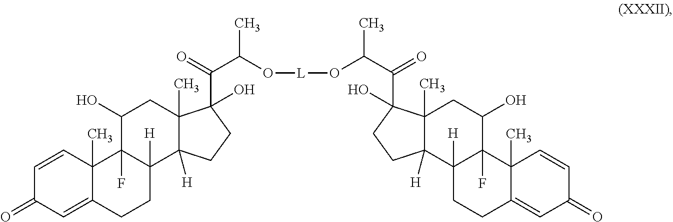

wherein L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (XXXII) can be formed from fluperolone.

In some embodiments, the steroid is a glucocorticoid steroid and the drug dimer is further described by the formula (XXXIII):

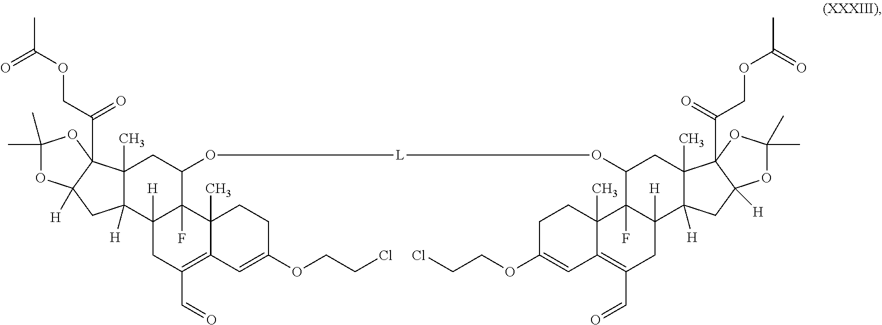

wherein L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (XXXIII) can be formed from formocortal.

In some embodiments, the steroid is a corticosteroid and the drug dimer is further described by the formula (XXXIV):

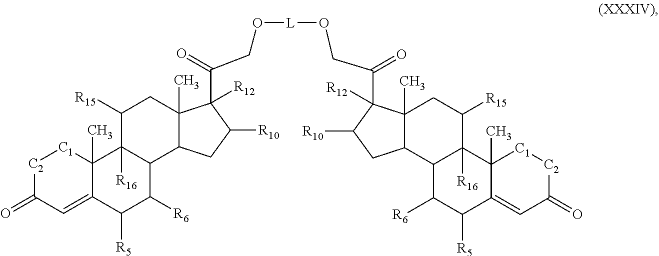

wherein the bond between C₁ and C₂ is a double or a single bond; R₁₆ represents H or a halogen atom; R₅ represents H, CH₃, or a halogen atom; R₁₂ represents H or a halogen atom; R₁₅ represents ═O or OH; R₁₂ and R₁₀ each, independently, represent —H, C₁₋₁₀ alkyl, —OH, —O-acyl, or R₁₂ and R₁₀ combine to form a cyclic acetal of formula (XVIII-a) wherein:

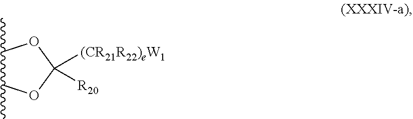

e is an integer from 0 to 6; R₂₀, R₂₁, and R₂₂ each, independently, represent H or C₁₋₁₀ alkyl; W₁ represents H or CH₃; L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (XXXIV) can be formed from a corticosteroid selected from the group consisting of alclometasone, beclomethasone, betamethasone, betamethasone valerate, budesonide, chloroprednisone, cloprednol, corticosterone, cortisone, desonide, desoximerasone, dexamethasone, diflorasone, diflucortolone, enoxolone, flucloronide, flumethasone, flunisolide, fluocinolone acetonide, fluocortolone, fluprednisolone, flurandrenolide, halometasone, hydrocortisone, hydrocortisone butyrate, meprednisone, methylprednicolone, paramethasone, prednisolone, prednisone, prednival, prednylidene, triamcinolone, and triamcinolone acetonide.

In any of the above formulas (II)-(XXXIV), O—(R^{A})—O can be a radical of a polyol formed from a cyclitol, and sugar alcohol, or glycerin; or O—(R^{A})—O can be a radical formed from an alkane diol (e.g., a C₁₋₁₀ alkane diol), diethylene glycol, triethylene glycol, tetraethylene glycol, or pentaethylene glycol.

In some embodiments, the steroid is a corticosteroid and the drug dimer is further described by the formula (XXXV):

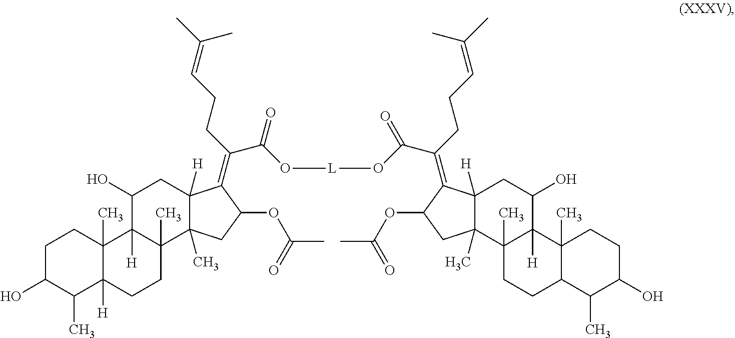

wherein L is —C(O)—(R^{A})—C(O)—, —(R^{A})—, or —C(O)—O—(R^{A})—O—C(O)— and R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms; or L is —O—(R^{A})—O— and R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from —O(CH₂CH₂O)ₙCH₂CH₂O—, —O(CH₂CH₂CH₂CH₂O)ₘCH₂CH₂CH₂CH₂O—, and —O(CH₂CH(CH₃)O)ₚCH₂CH(CH₃)O—; and n, m, and p are integers from 1 to 10. The drug dimer of formula (XXXV) can be formed from fusidic acid.

In some embodiments, the steroid is a corticosteroid and the drug dimer is further described by the formula (XXXVI):

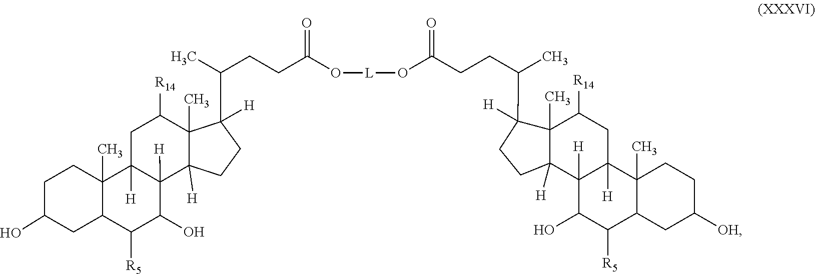

wherein R₅ represents H or C₁₋₆alkyl; R₁₄ represents H or OH; and L is —C(O)—(R^{A})—C(O)—, —(R^{A})—, or —C(O)—O—(R^{A})—O—C(O)— and R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms; or L is —O—(R^{A})—O— and R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from —O(CH₂CH₂O)ₙCH₂CH₂O—, —O(CH₂CH₂CH₂CH₂O)ₘCH₂CH₂CH₂CH₂O—, and —O(CH₂CH(CH₃)O)ₚCH₂CH(CH₃)O—; and n, m, and p are integers from 1 to 10. The drug dimer of formula (XXXVI) can be formed from chenodeoxycholic acid, ursodeoxycholic acid, or obeticholic acid.

In some embodiments, the steroid is an anti-angiogenic steroid or an intraocular pressure (IOP) lowering steroid, and the drug dimer is further described by the formula (XXXVII):

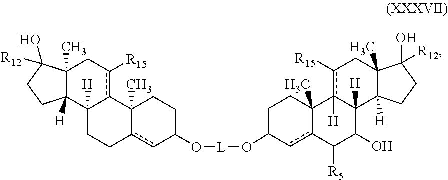

wherein R₁₂ represents —C(═O)CH₂OC(═O)CH₃, —C(═O)CH₂OH, or —C(═O)CH₃; R₁₅ represents H or OH; and L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₚCH₂CH₂O—, O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (XXXVII) can be formed from anecortave acetate, anecortave, 11-epicortisol, 17α-hydroxyprogesterone, tetrahydrocortexolone, or tetra hydrocortisol.

In some embodiments, the steroid is an anti-angiogenic steroid or an intraocular pressure (IOP) lowering steroid, and the drug dimer is further described by the formula (XXXVIII):

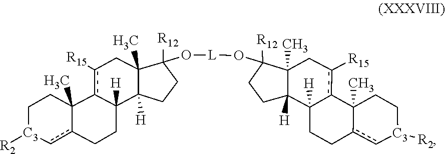

wherein the bond between C₃ and R₂ is a single or a double bond; R₂ represents OH or ═O; R₁₂ represents —C(═O)CH₂OC(═O)CH₃, —C(═O)CH₂OH, or —C(═O)CH₃, R₁₅ represents H or OH; and L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₚCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (XXXVIII) can be formed from anecortave acetate, anecortave, 11-epicortisol, 17α-hydroxyprogesterone, tetrahydrocortexolone, or tetra hydrocortisol.

In some embodiments, the steroid is an anti-angiogenic steroid or an intraocular pressure (IOP) lowering steroid, and the drug dimer is further described by the formula (XXXIX):

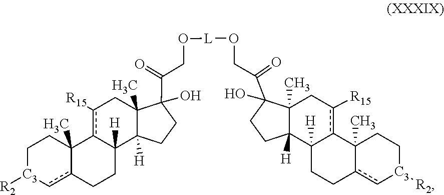

wherein the bond between C₃ and R₂ is a single or a double bond; R₂ represents OH or ═O; R₁₅ represents H or OH; and L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (XXXIX) can be formed from anecortave, 11-epicortisol, tetra hydrocortexolone, or tetra hydrocortisol.

In some embodiments, the steroid is an anti-angiogenic steroid or an intraocular pressure (IOP) lowering steroid, and the drug dimer is further described by the formula (XL):

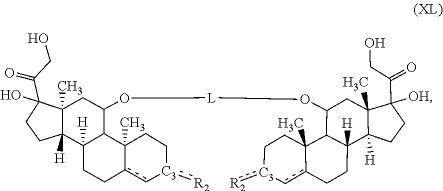

wherein the bond between C₃ and R₂ is a single or a double bond; R₂ represents OH or ═O; and L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (XL) can be formed from 11-epicortisol or tetra hydrocortisol.

In some embodiments, the steroid is a benign steroid and the drug dimer is further described by the formula (XLI):

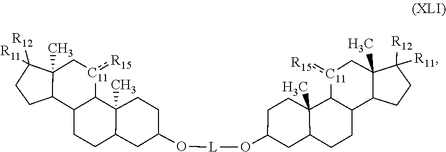

wherein the bond between C_{H} and R₁₅ is a single or a double bond; R₁₁ represents H, OH, —C(═O)CH₂OH, or —C(═O)CH₃; R₁₂ represents H, OH, —C(═O)CH₂OH, or —C(═O)CH₃; R₁₅ represents H, ═O, or OH; and L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (XLI) can be formed from tetra hydrocortisone, tetrahydrodeoxycortisol, tetra hydrocorticosterone, 5α-dihydrocorticosterone, or 5α-dihydropregesterone.

In some embodiments, the steroid is a benign steroid and the drug dimer is further described by the formula (XLII):

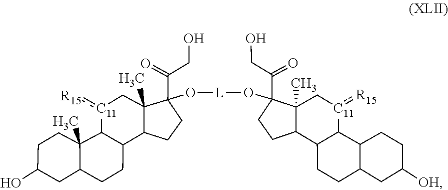

wherein the bond between CH and R₁₅ is a single or a double bond; R₁₅ represents H or ═O; and L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (XLII) can be formed from tetra hydrocortisone, or tetrahydrodeoxycortisol.

In some embodiments, the steroid is a benign steroid and the drug dimer is further described by the formula (XLIII):

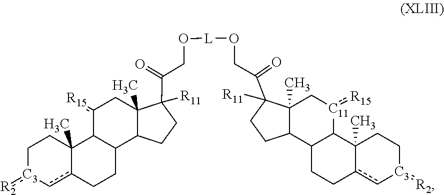

wherein the bond between C₃ and R₂, and C_{H} and R₁₅ is a single or a double bond; R₂ represents OH or ═O; R₁₁ represents H, or OH; R₁₅ represents H, ═O, or OH; and L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (XLIII) can be formed from tetra hydrocortisone, tetrahydrodeoxycortisol, tetrahydrocorticosterone, or 5α-di hydrocorticosterone.

In some embodiments, the steroid is a benign steroid and the drug dimer is further described by the formula (XLIV):

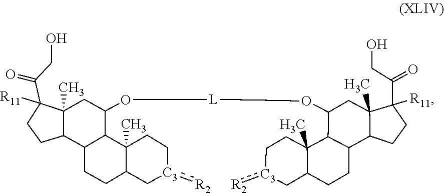

wherein the bond between C₃ and R₂ is a single or a double bond; R₂ represents OH or ═O; R₁₁ represents H or OH; and L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (XLIV) can be formed from tetra hydrocortisone, tetrahydrocorticosterone, or 5α-dihydrocorticosterone.

In some embodiments, the steroid is a cholic acid-related bile acid steroid and the drug dimer is further described by the formula (XLV):

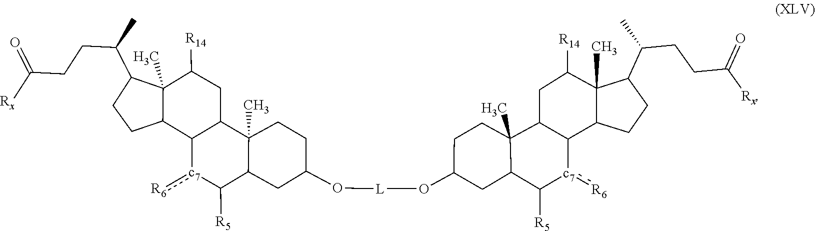

wherein the bond between C₇ and R₆, and C₁₂ and R₁₄ is a single or a double bond; Rx represents OH, —NHCH₂C(═O)OH, or —NHCH₂CH₂SO₂OH; R₂ represents OH or ═O; R₅ represents H or OH; R₆ represents H, ═O, or OH; R₁₄ represents H, ═O, or OH; and L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (XLV) can be formed from deoxycholic acid, apocholic acid, dehydrocholic acid, glycochenodeoxycholic acid, glycocholic acid, glycodeoxycholic acid, hyodeoxycholic acid, lithocholic acid, α-muricholic acid, β-muricholic acid, γ-muricholic acid, ω-muricholic acid, taurochenodeoxycholic acid, taurocholic acid, taurodeoxycholic acid, taurolithocholic acid, or tauroursodeoxycholic acid.

In some embodiments, the steroid is a cholic acid-related bile acid steroid and the drug dimer is further described by the formula (XLVI):

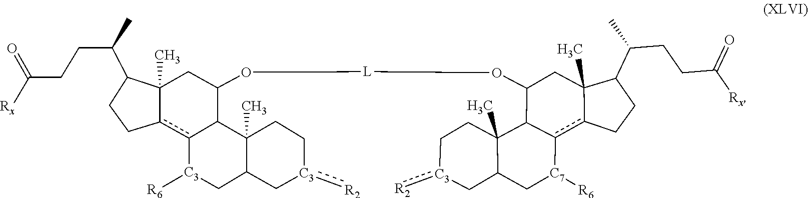

wherein the bond between C₃ and R₂, and C₇ and R₆ is a single or a double bond; Rx represents OH, —NHCH₂C(═O)OH, or —NHCH₂CH₂SO₂OH; R₂ represents OH or ═O; R₆ represents H, ═O, or OH; and L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (XLVI) can be formed from deoxycholic acid, apocholic acid, dehydrocholic acid, glycocholic acid, glycodeoxycholic acid, taurocholic acid, or taurodeoxycholic acid.

In some embodiments, the steroid is a cholic acid-related bile acid steroid and the drug dimer is further described by the formula (XLVII):

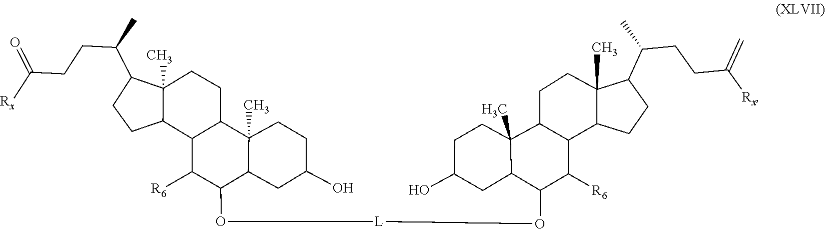

wherein R₆ represents H or OH; and L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (XLVII) can be formed from hyodeoxycholic acid, α-muricholic acid, β-muricholic acid, γ-muricholic acid, or ω-muricholic acid.

In some embodiments, the steroid is a cholic acid-related bile acid steroid and the drug dimer is further described by the formula (XLVIII):

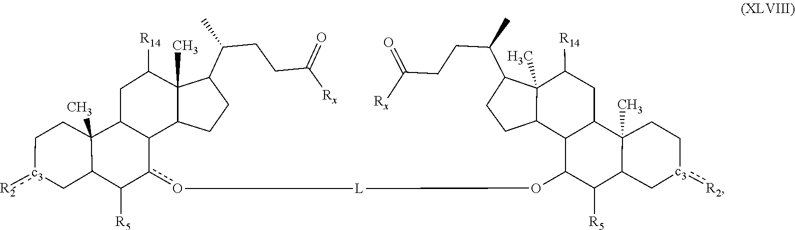

wherein the bond between C₃ and R₂, and C₁₂ and R₁₄ is a single or a double bond; Rx represents OH, —NHCH₂C(═O)OH, or —NHCH₂CH₂SO₂OH; R₂ represents OH or ═O; R₅ represents H or OH; R₁₄ represents H, ═O, or OH; and L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (XLVIII) can be formed from dehydrocholic acid, glycochenodeoxycholic acid, glycocholic acid, α-muricholic acid, β-muricholic acid, γ-muricholic acid, ω-muricholic acid, taurochenodeoxycholic acid, taurocholic acid, or tauroursodeoxycholic acid.

In some embodiments, the steroid is a cholic acid-related bile acid steroid and the drug dimer is further described by the formula (XLIX):

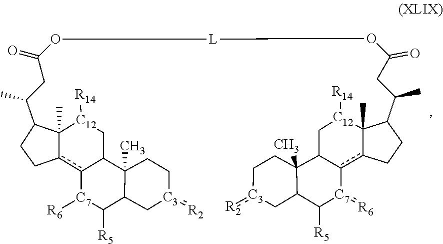

wherein the bond between C₃ and R₂, C₇ and R₆, and C₁₂ and R₁₄ is a single or a double bond; R₂ represents OH or ═O; R₅ represents H or OH; R₆ represents H, ═O, or OH; R₁₄ represents H, ═O, or OH; and L is —C(O)—(R^{A})—C(O)—, —(R^{A})—, or —C(O)—O—(R^{A})—O—C(O)— and R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms; or L is —O—(R^{A})—O— and R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from —O(CH₂CH₂O)ₙCH₂CH₂O—, —O(CH₂CH₂CH₂CH₂O)ₘCH₂CH₂CH₂CH₂O—, and —O(CH₂CH(CH₃)O)ₚCH₂CH(CH₃)O—; and n, m, and p are integers from 1 to 10. The drug dimer of formula (XLIX) can be formed from deoxycholic acid, apocholic acid, dehydrocholic acid, hyodeoxycholic acid, lithocholic acid, α-muricholic acid, β-muricholic acid, γ-muricholic acid, or ω-muricholic acid.

In some embodiments, the steroid is a cholic acid-related bile acid steroid and the drug dimer is further described by the formula (L):

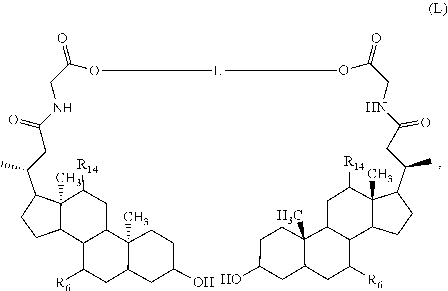

wherein R₆ represents H or OH; R₁₄ represents H or OH; and L is —C(O)—(R^{A})—C(O)—, —(R^{A})—, or —C(O)—O—(R^{A})—O—C(O)— and R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms; or L is —O—(R^{A})—O— and R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from —O(CH₂CH₂O)ₙCH₂CH₂O—, —O(CH₂CH₂CH₂CH₂O)ₘCH₂CH₂CH₂CH₂O—, and O(CH₂CH(CH₃)O)ₚCH₂CH(CH₃)O—; and n, m, and p are integers from 1 to 10. The drug dimer of formula (L) can be formed from glycochenodeoxycholic acid, glycocholic acid, or glycodeoxycholic acid.

In some embodiments, the steroid is a steroid metabolite and the drug dimer is further described by the formula (LI):

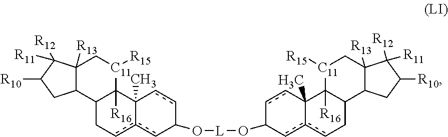

wherein the bond between C_{H} and R₁₅ is a single or a double bond; R₂ represents OH or ═O; R₁₀ represents H or OH; R₁₁ represents H, OH, —C(═O)CH₂OH, —C(═O)OH, —C(═O)CH₂OH, or —C(═O)CH₃; R₁₂ represents H, OH, —C(═O)CH₂OH, —C(═O)OH, —C(═O)CH₂OH, or —C(═O)CH₃; R₁₃ represents —CH₂OH or —CH₃; R₁₅ represents H, OH, or ═O; R₁₆ represents H or F; and L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (LI) can be formed from tetrahydrotriamcinolone, cortienic acid, 11-dehydrocorticosterone, 11β-hydroxypregnenolone, ketoprogesterone, 17-hydroxypregnenolone, 17,21-di hydroxypregnenolone, 18-hydroxycorticosterone, deoxycortisone, 21-hydroxypregnenolone, or progesterone.

In some embodiments, the steroid is a steroid metabolite and the drug dimer is further described by the formula (LII):

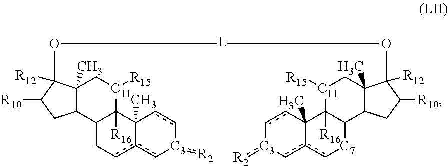

wherein the bond between C₃ and R₂, and C_{H} and R₁₅ is a single or a double bond; R₂ represents OH or ═O; R₁₀ represents H or OH; R₁₂ represents —C(═O)CH₂OH, —C(═O)OH, —C(═O)CH₂OH, or —C(═O)CH₃; R₁₅ represents H, OH, or ═O; R₁₆ represents H or F; and L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (LII) can be formed from tetrahydrotriamcinolone, cortienic acid, 17-hydroxypregnenolone, 17,21-dihydroxypregnenolone, or deoxycortisone.

In some embodiments, the steroid is a steroid metabolite and the drug dimer is further described by the formula (LIII):

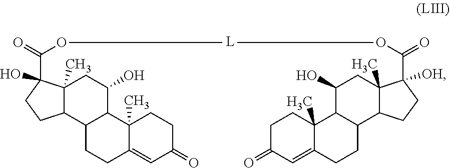

wherein L is —C(O)—(R^{A})—C(O)—, —(R^{A})—, or —C(O)—O—(R^{A})—O—C(O)— and R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms; or L is —O—(R^{A})—O— and R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from —O(CH₂CH₂O)ₙCH₂CH₂O—, —O(CH₂CH₂CH₂CH₂O)ₘCH₂CH₂CH₂CH₂O—, and —O(CH₂CH(CH₃)O)ₚCH₂CH(CH₃)O—; and n, m, and p are integers from 1 to 10. The drug dimer of formula (LIII) can be formed from cortienic acid.

In some embodiments, the steroid is a steroid metabolite and the drug dimer is further described by the formula (LIV):

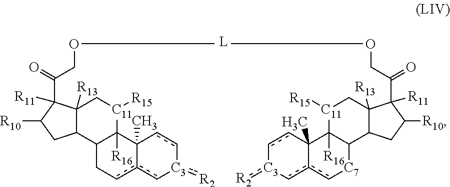

wherein the bond between C₃ and R₂, and C_{H} and R₁₅ is a single or a double bond; R₂ represents OH or ═O; R₁₀ represents H or OH; R₁₁ represents H or OH; Rn represents H, —CH₂OH, or —CH₃; R₁₅ represents H, OH, or ═O; R₁₆ represents H or F; and L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (LIV) can be formed from tetrahydrotriamcinolone, 11-dehydrocorticosterone, 17,21-di hydroxypregnenolone, 18-hydroxycorticosterone, or 21-hydroxypregnenolone.

In some embodiments, the steroid is a steroid metabolite and the drug dimer is further described by the formula (LV):

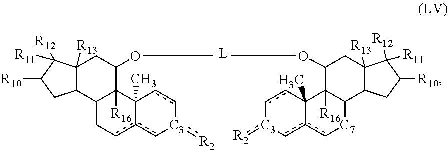

wherein the bond between C₃ and R₂ is a single or a double bond; R₂ represents OH or ═O; R₁₀ represents H or OH; R₁₁ represents H or OH; R₁₂ represents —C(═O)CH₂OH, —C(═O)OH, —C(═O)CH₂OH, or —C(═O)CH₃; R₁₃ represents H, —CH₂OH, or —CH₃; R₁₆ represents H or F; and L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₚCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (LV) can be formed from tetrahydrotriamcinolone, cortienic acid, 11-dehydrocorticosterone, 11β-hydroxypregnenolone, ketoprogesterone, 18-hydroxycorticosterone, or deoxycortisone.

In some embodiments, the steroid is a steroid metabolite and the drug dimer is further described by the formula (LVI):

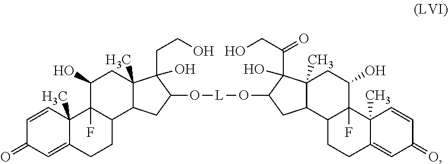

wherein L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (LVI) can be formed from tetrahydrotriamcinolone.

In some embodiments, the steroid is a steroid metabolite and the drug dimer is further described by the formula (LVII):

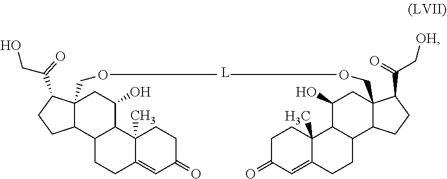

wherein L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (LVII) can be formed from 18-hydroxycorticosterone.

In some embodiments, the steroid is a cholesterol-derivative and the drug dimer is further described by the formula (LVIII):

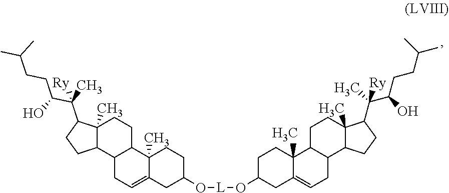

wherein Ry represents H or OH; and L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (LVIII) can be formed from 22R-hydroxycholesterol or 20α-22R-dihydroxycholesterol.

In some embodiments, the steroid is a cholesterol-derivative and the drug dimer is further described by the formula (LIX):

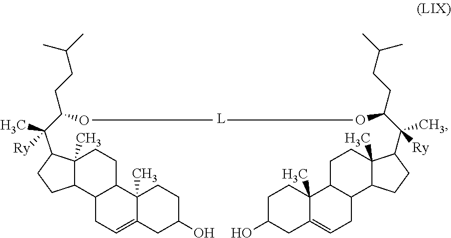

wherein Ry represents H or OH; and L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (LIX) can be formed from 22R-hydroxycholesterol or 20α-22R-dihydroxycholesterol.

In some embodiments, the steroid is a cholesterol-derivative and the drug dimer is further described by the formula (LX):

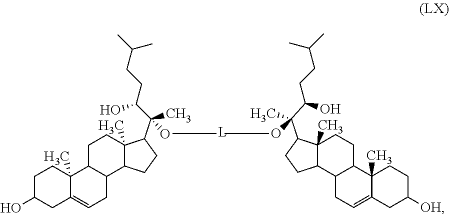

wherein L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (LX) can be formed from 20α-22R-di hydroxycholesterol.

In some embodiments, the steroid is a neurosteroid and the drug dimer is further described by the formula (LXI):

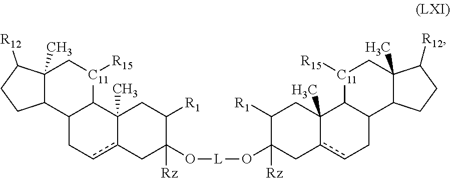

wherein the bond between C_{H} and R₁₅ is a single or a double bond; Rz represents H or —CH₃; R₁ represents H or —OCH₂CH₃; R₂ represents OH or ═O; R₁₂ represents —OH, —C(═O)CH₃, —C(═O)CH₂OH, or —CH(CH₃)(CH₂)₂CH(OH)CH(CH₃)₂; R₁₅ represents H, —N(CH₃)₂, or ═O; and L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (LXI) can be formed from alphaxalone, alphadolone, hydroxydione, minaxolone, tetrahydrodeoxycorticosterone, allopregnanolone, pregnanolone, ganoxolone, 3α-androstanediol, epipregnanolone, isopregnanolone, or 24(S)-hydroxycholesterol.

In some embodiments, the steroid is a neurosteroid and the drug dimer is further described by the formula (LXII):

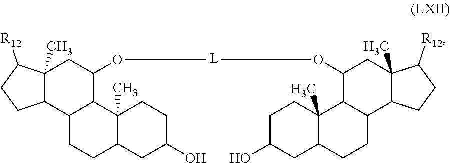

wherein R₁₂ represents —C(═O)CH₃, or —C(═O)CH₂OH; and L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (LXII) can be formed from alphaxalone or alphadolone.

In some embodiments, the steroid is a neurosteroid and the drug dimer is further described by the formula (LXIII):

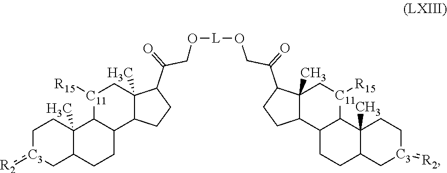

wherein the bond between C₃ and R₂, and CH and R₁₅ is a single or a double bond; R₂ represents OH or ═O; R₁₅ represents H or ═O; and L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (LXIII) can be formed from alphadolone, hydroxydione, or tetra hydrodeoxycorticosterone.

In some embodiments, the steroid is a neurosteroid and the drug dimer is further described by the formula (LXIV):

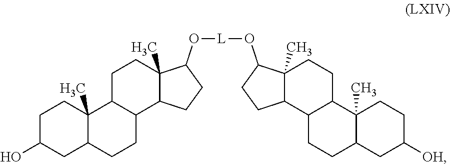

wherein L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (LXIV) can be formed from 3α-androstanediol.

In some embodiments, the steroid is a neurosteroid and the drug dimer is further described by the formula (LXV):

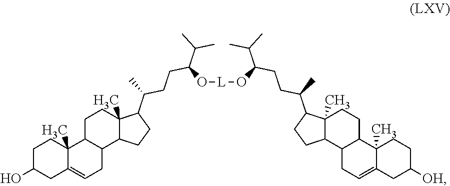

wherein L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (LXV) can be formed from 24(S)-hydroxycholesterol.

In some embodiments, the steroid is a pheromone and the drug dimer is further described by the formula (LXVI):

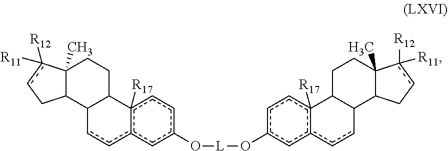

wherein R₂ represents OH or ═O; R₁₁ represents H, —C(═O)CH₃, —OC(═O)(CH₂)₄CH₃, or is absent; R₁₂ represents H, —C(═O)CH₃, —OC(═O)(CH₂)₄CH₃, or is absent; R₁₇ represents CH₃ or is absent; and L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (LXVI) can be formed from androstadienol, androstadienone, androstenol, androstenone, estratetraenol, 5-dehydroprogesterone, 6-dehydro-retroprogesterone, allopregnanolone, or hydroxyprogesterone caproate.

In some embodiments, the steroid is a progestin and the drug dimer is further described by the formula (LXVII):

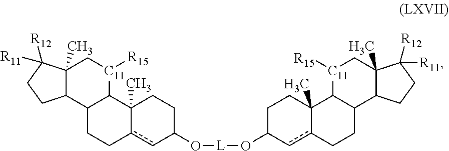

wherein the bond between C_{H} and R₁₅ is a single or a double bond; R₂ represents OH or ═O; R₁₁ represents H, OH, —CH(OH)CH₃, —C(═O)CH₂OH, —C(═O)CH₃, or —CH(OH)CH₂OH; R₁₂ represents H, OH, —CH(OH)CH₃, —C(═O)CH₂OH, —C(═O)CH₃, or —CH(OH)CH₂OH; R₁₅ represents H, ═O, or OH; and L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (LXVII) can be formed from allopregnone-3α,20α-diol, allopregnone-3β,20β-diol, allopregnane-3β,21-diol-11,20-dione, allopregnane-3β,17α-diol-20-one, 3,20-allopregnanedione, 3β,11β,17α,20β,21-pentol, allopregnane-3β,17α, 20β,21-tetrol, allopregnane-3α,11β,17α, 21-tetrol-20-one, allopregnane-3β,11β,17α, 21-tetrol-20-one, allopregnane-3β,17α,20β-triol, allopregnane-3β,17α, 21-triol-11,20-dione, allopregnane-3β,11β,21-triol-20-one, allopregnane-3β,17α, 21-triol-20-one, allopregnane-3α-ol-20-one; allopregnane-3β-ol-20-one, pregnanediol, 3,20-pregnanedione, 4-pregnene-20,21-diol-3,11-dione, 4-pregnene-11β,17α,20β,21-tetrol-3-one, 4-pregnene-17α, 20β,21-triol-3,11-dione, 4-pregnene-17α,20β,21-triol-3-one, or pregnenolone.

In some embodiments, the steroid is a progestin and the drug dimer is further described by the formula (LXVIII):

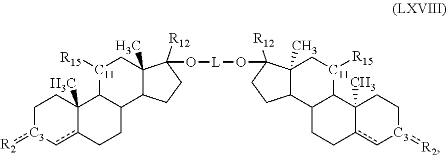

wherein the bond between C₃ and R₂, and CH and R₁₅ is a single or a double bond; R₂ represents OH or ═O; R₁₂ represents H, OH, —CH(OH)CH₃, —C(═O)CH₂OH, —C(═O)CH₃, or —CH(OH)CH₂OH; R₁₅ represents H, ═O, or OH; and L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (LXVIII) can be formed from allopregnane-3β,17α-diol-20-one, 3,20-allopregnanedione,3β,11β,17α, 20β,21-pentol, allopregnane-3β,17α, 20β,21-tetrol, allopregnane-3α,11β,17α, 21-tetrol-20-one, allopregnane-3β,11β,17α, 21-tetrol-20-one, allopregnane-3β,17α, 20β-triol, allopregnane-3β,17α, 21-triol-11,20-dione, allopregnane-3β,17α, 21-triol-20-one, 4-pregnene-11β,17α, 20β,21-tetrol-3-one, 4-pregnene-17α, 20β,21-triol-3,11-dione, or 4-pregnene-17α, 20β,21-triol-3-one.

In some embodiments, the steroid is a progestin and the drug dimer is further described by the formula (LXIX):

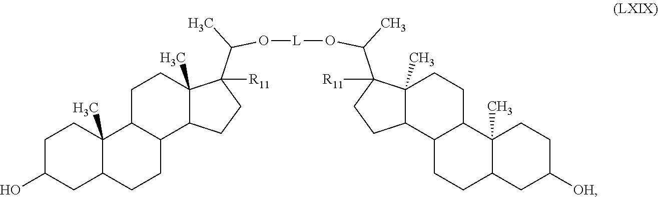

wherein R₁₁ represents H, OH, —CH(OH)CH₃, —C(═O)CH₂OH, —C(═O)CH₃, or —CH(OH)CH₂OH; R₁₅ represents H or OH; and L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (LXIX) can be formed from allopregnone-3α,20α-diol, allopregnone-3β,20β-diol or allopregnane-3β,17α, 20β-triol.

In some embodiments, the steroid is a progestin and the drug dimer is further described by the formula (LXX):

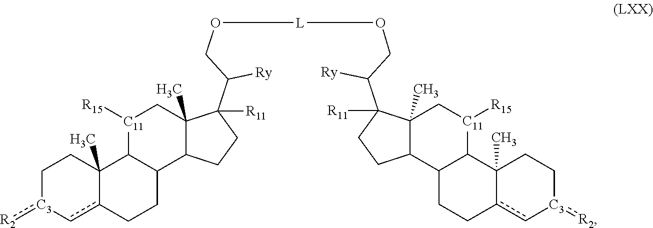

wherein the bond between C₃ and R₂, and C_{H} and R₁₅ is a single or a double bond; Ry represents OH or ═O; R₂ represents OH or ═O; R₁₁ represents H, OH, —CH(OH)CH₃, —C(═O)CH₂OH, —C(═O)CH₃, or —CH(OH)CH₂OH; R₁₂ represents H, OH, —CH(OH)CH₃, —C(═O)CH₂OH, —C(═O)CH₃, or —CH(OH)CH₂OH; R₁₅ represents H, ═O, or OH; and L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (LXX) can be formed from allopregnane-3β,21-diol-11,20-dione, 3,20-allopregnanedione,3β,11β,17α, 20β,21-pentol, allopregnane-3β,17α, 20β,21-tetrol, allopregnane-3α,11β,17α, 21-tetrol-20-one, allopregnane-3β,11β,17α, 21-tetrol-20-one, allopregnane-3β,17α, 21-triol-11,20-dione, allopregnane-3β,11β,21-triol-20-one, allopregnane-3β,17α, 21-triol-20-one, 4-pregnene-20,21-diol-3,11-dione, 4-pregnene-11β,17α, 20β,21-tetrol-3-one, 4-pregnene-17α, 20β,21-triol-3,11-dione, or 4-pregnene-17α, 20β,21-triol-3-one.

In some embodiments, the steroid is a progestin and the drug dimer is further described by the formula (LXXI):

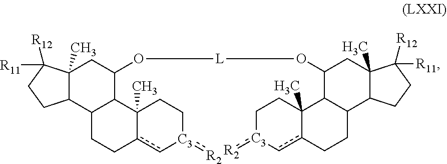

wherein the bond between C₃ and R₂ is a single or a double bond; R₂ represents OH or ═O; R₁₁ represents H, OH, —CH(OH)CH₃, —C(═O)CH₂OH, —C(═O)CH₃, or —CH(OH)CH₂OH; R₁₂ represents H, OH, —CH(OH)CH₃, —C(═O)CH₂OH, —C(═O)CH₃, or —CH(OH)CH₂OH; and L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (LXXI) can be formed from allopregnane-3β,21-diol-11,20-dione, 3,20-allopregnanedione,3β,11β,17α, 20β,21-pentol, allopregnane-3α,11β,17α, 21-tetrol-20-one, allopregnane-3β,11β,17α, 21-tetrol-20-one, allopregnane-3β,17α, 21-triol-11,20-dione, allopregnane-3β,11β,21-triol-20-one, 4-pregnene-20,21-diol-3,11-dione, 4-pregnene-11β,17α, 20β,21-tetrol-3-one, or 4-pregnene-17α, 20β,21-triol-3,11-dione.

In some embodiments, the steroid is a progestin and the drug dimer is further described by the formula (LXXII):

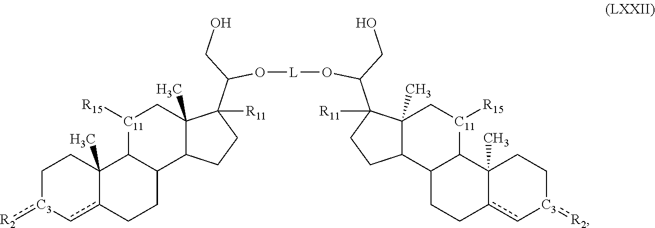

wherein the bond between C₃ and R₂, and CH and R₁₅ is a single or a double bond; R₂ represents OH or ═O; R₁₁ represents H or OH; R₁₅ represents H, ═O, or OH; and L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (LXXII) can be formed from 3,20-allopregnanedione,3β,11β,17α, 20β,21-pentol, allopregnane-3β,17α, 20β,21-tetrol, 4-pregnene-20,21-diol-3,11-dione, 4-pregnene-11β,17α, 20β,21-tetrol-3-one, 4-pregnene-17α, 20β,21-triol-3,11-dione, or 4-pregnene-17α, 20β,21-triol-3-one.

In some embodiments, the steroid is other steroid and the drug dimer is further described by the formula (LXXIII):

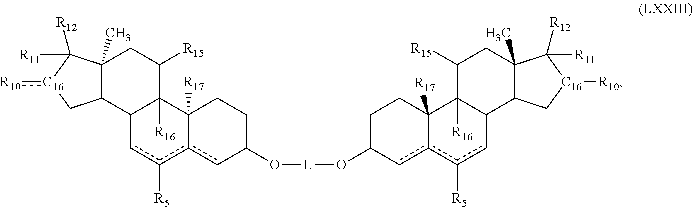

wherein the bond between C₁₆ and R₁₀ is a single or a double bond; R₂ represents OH or ═O; R₅ represents H, Cl, or —CH₃; R₁₀ represents H or ═CH₂; R₁₁ represents H, OH, —CH₃, —C(═O)CH₃, —C(═O)CH₂OC(═O)CH₃, or —OC(═O)CH₃; R₁₂ represents H, OH, —CH₃, —C(═O)CH₃, —C(═O)CH₂OC(═O)CH₃, or —OC(═O)CH₃; R₁₅ represents H or OH; R₁₆ represents F or H; R₁₇ represents H or —CH₃; and L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (LXXIII) can be formed from flugestone, prebediolone, chlormadinone acetate, medrogestone, or segesterone acetate.

In some embodiments, the steroid is other steroid and the drug dimer is further described by the formula (LXXIV):

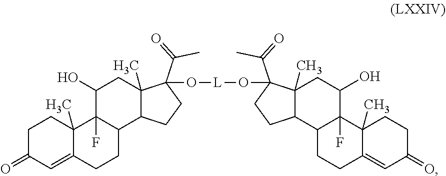

wherein L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (LXXIV) can be formed from flugestone.

In some embodiments, the steroid is another steroid and the drug dimer is further described by the formula (LXXV):

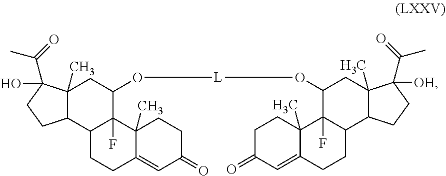

wherein L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (LXXV) can be formed from flugestone.

In some embodiments, the steroid is a mineralocorticoid steroid, and the drug dimer is further described by the formula (LXXVI):

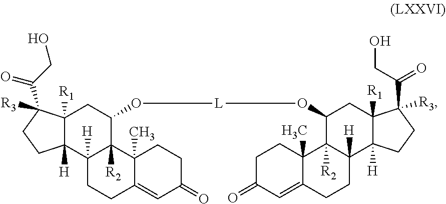

wherein R₁ is C(O)H or CH₃; R₂ represents H or F; R₃ represents H or OH; and L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (LXXVI) can be formed from fludrocortisone or aldocortisone.

In some embodiments, the steroid is a mineralocorticoid steroid, and the drug dimer is further described by the formula (LXXVII):

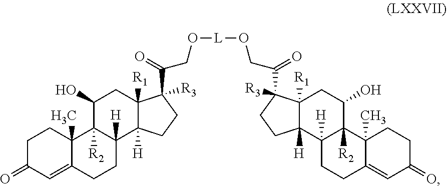

wherein R₁ is C(O)H or CH₃; R₂ represents H or F; R₃ represents H or OH; and L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (LXXVII) can be formed from fludrocortisone or aldocortisone.

In some embodiments, the steroid is a mineralocorticoid steroid, and the drug dimer is further described by the formula (LXXVIII):

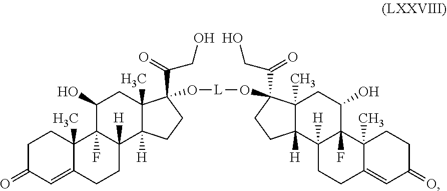

wherein L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (LXXVIII) can be formed from fludrocortisone.

### Second Agents

In some embodiments, the second agent is a therapeutic agent. In some embodiments, the therapeutic agent is any suitable therapeutic agent such as, for example, a small or large molecule (e.g., a biologic).

Provided in some embodiments herein is an article comprising a second agent that has a extended, a sustained, an immediate, and/or a delayed release profile (e.g., depending upon the design of the article). By way of non-limiting example, the second agent can be, but is not limited to, an analgesic, opioid, antimicrobial, anti-proliferative, kinase inhibitor, steroid, non-steroidal anti-inflammatory drug (NSAID), immunomodulatory agent, or prostaglandin or a combination of more than one free drug. Any second agent (e.g., or therapeutic agent) described herein, or its pharmaceutically acceptable salt form, can be used in an article provided herein.

Provided in certain instances herein the second agent is an analgesic selected from lidocaine, bupivacaine, mepivacaine, ropivacaine, tetracaine, etidocaine, chloroprocaine, prilocaine, procaine, benzocaine, dibucaine, dyclonine hydrochloride, pramoxine hydrochloride, benzocaine, proparacaine, and pharmaceutically acceptable salts thereof. Articles including an analgesic can be useful for treating, managing, or reducing the risk of, pain.

In some embodiments, provided herein the second agent is an opioid agonist. In some embodiments, the second agent is an opioid antagonist. In some embodiments of any of the articles and methods provided herein the second agent is an opioid selected from buprenorphine, nor-buprenorphine, fentanyl, methadone, levorphanol, morphine, hydromorphone, oxymorphone codeine, oxycodone, hydrocodone, and their pharmaceutically acceptable salts. Articles including an opioid can be useful for treating, or reducing the risk of, pain.

In certain embodiments, the second agent is an antimicrobial agent, such as, by way of non-limiting example, an anti-bacterial, antifungal, or antiviral agent. In some embodiments, a formulation provided herein comprises a therapeutic agent, such as, for example, a β-lactam antibiotic (e.g., cephalosporins, carbapenems, monobactams, penicillins, penems, carbacephems, and oxacephems). In some embodiments, the therapeutic agent is a cephalosporin, or a salt thereof (e.g., cefepime, cefalonium, cephaloridine, cefpimizole, ceftazidime, cefluprenam, cefozopran, cefpirome, cefquinome, cefmepidium, ceftaroline, ceftaroline fosamil, and salts thereof). In some embodiments, the therapeutic agent is cefepime, or a salt thereof. In still other embodiments, the drug is a carbapenem, or a salt thereof (e.g., meropenem or ertapenem, or a salt thereof).

Cephalosporins are a class of β-lactam antibiotics with broad-spectrum activity against Gram-positive and Gram-negative bacteria. Many cephalosporins share a core structure shown by formula (A), where R₁ and R₂ can be any substituent.

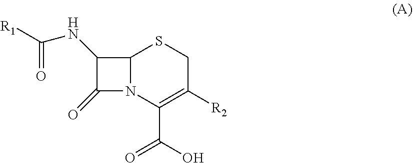

In some embodiments, an article provided herein comprises, for example, cefepime (Maxipime®, Maxcef®, Cepimax®, Cepimex®, and Axepim®; 1-[[6R,7R)-7-[2-(2-amino-4-thiazolyl)glyoxylamido]-2-carboxy-8-oxo-5-thia-1-azabicyclo[4.2.0]oct-2-en-3-yl]methyl]-1-methlypyrrolidinium chloride, 72-(Z)—(O-methyloxime), monohydrochloride, monohydrate), cefotaxime (Claforan®, Taxime®; (6R,7R,Z)-3-(acetoxymethyl)-7-(2-(2-aminothiazol-4-yl)-2-(methoxyimino)acetamido)-8-oxo-5-thia-1-azabicyclo[4.2.0]oct-2-ene-2-carboxylic acid), ceftriaxone)(Rocephin®, cefacetrile (cephacetrile), cefadroxil (cefadroxyl; duricef), cefoselis, cephalexin (cephalexin; Keflex®), cefaloglycin (cephaloglycin), cefalonium (cephalonium), cephaloridine (cefaloridine), cefalotin (cephalothin; Keflin®), cefapirin (cephapirin; Cefadryl®), cefatrizine, cefazaflur, cefazedone, cefazolin (cephazolin; Ancef®, Kefzol®), cefradine (cephradine; Velosef®), cefroxadine, ceftezole, cefaclor (Ceclor®, Distaclor®, Keflor®, Raniclor®), cefonicid (Monocid®), cefprozil (cefproxil; Cefzil®), cefuroxime (Zinnat®, Zinacef®, Ceftin®, Biofuroksym®), cefuroxime axetil (Ceftin®), cefuzonam, cefbuperazone, cefmetazole) (Zefazone®, cefminox, cefotetan (Apatef®, Cefotan®), cefoxitin (Mefoxin®), cefcapene, cefdaloxime, cefdinir (Omnicef®, Kefnir®), cefditoren, cefetamet, cefixime (Suprax®), cefmenoxime, cefodizime, cefpimizole, cefpodoxime (Vantin®, Pecef®), cefteram, ceftibuten (Cedax®), ceftiofur, ceftiolene, ceftizoxime (Cefizox®), cefoperazone (Cefobid®), ceftazidime (Fortum®, Fortaz®), cefclidine, cefluprenam, cefoselis, cefozopran, cefpirome (Cefrom®), cefquinome, ceftobiprole (Medocaril®), cefaclomezine, cefaloram, cefaparole, cefcanel, cefedrolor, cefempidone, cefetrizole, cefivitril, cefmatilen, cefmepidium, cefovecin, cefoxazole, cefrotil, cefsumide, ceftaroline, ceftaroline fosamil (Teflaro™), ceftioxide, cefuracetime, ceftobiprole, or other derivatives or analogs of 7-aminocephalosporanic acid. The structures of cefepime, cefotaxime, and ceftriaxone are shown below.

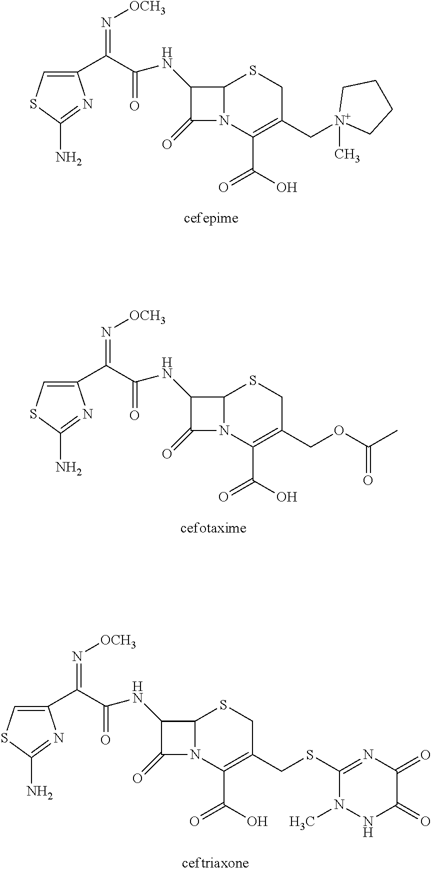 [SMILES: CO/N=C(\C(=O)NC1C(=O)N2C(C(=O)O)=C(C[N+]3(C)CCCC3)CSC12)c1csc(N)n1 ; CO/N=C(\C(=O)NC1C(=O)N2C(C(=O)O)=C(COC(C)=O)CSC12)c1csc(N)n1 ; CO/N=C(\C(=O)NC1C(=O)N2C(C(=O)O)=C(CSc3nc(=O)c(=O)[nH]n3C)CSC12)c1csc(N)n1]

Carbapenems are a class of beta-lactam antibiotics with broad spectrum antibacterial activity. The carbapenems are structurally very similar to the penicillins, but the sulfur atom in the structure has been replaced with a carbon atom, as shown in formula (B) below:

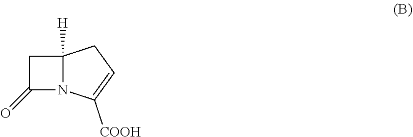 [SMILES: O=C(O)C1=CC[C@@H]2CC(=O)N12]

This structural modification renders them highly resistant to beta-lactamases. Carbapenem antibiotics were originally developed from thienamycin, a naturally-derived product of Streptomyces cattleya. Carbapenems which can be used in the articles and methods of the invention include, for example, imipenem, meropenem, ertapenem, doripenem, panipenem/betamipron, biapenem, and PZ-601. Structures of meropenem and ertapenem are given below:

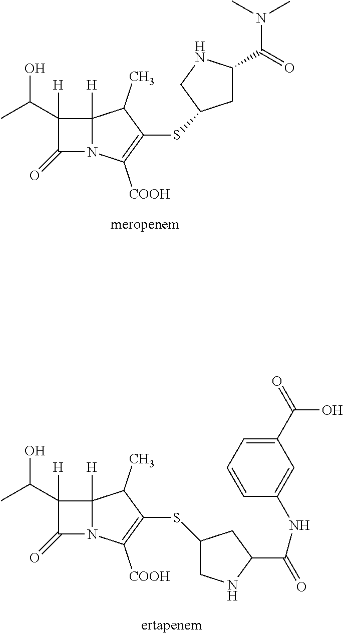

Antimicrobials which can be used in the articles and methods of the invention include, for example, monobactam antibiotics (e.g., aztreonam), polymyxin antibiotics (e.g., polymyxin B), rifamycin antibiotics (e.g., rifampin, rifabutin, rifapentine, rifalazil), quinolone antibiotics (e.g., flumequine, nalidixic acid, oxolinic acid, piromidic acid, pipemidic acid, rosoxacin, ciprofloxacin, enoxacin, lomefloxacin, nadifloxacin, norfloxacin, ofloxacin, pefloxacin, rufloxacin, balofloxacin, gatifloxacin, grepafloxacin, levafloxacin, rnoxifloxacin, pazufloxacin, sparfloxacin, temafloxacin, tosuflaxacin, besifloxacin, delafloxacin, clinafloxacin, gemifloxacin, prulifloxacin, sitafloxacin, troyafloxacin), fluoroquinolone antibiotics (e.g., delafloxacin), sulfonamide antibiotics (e.g., sulfarnethizole, suifamethoxazole, trimethoprim-sulfamethoxazole), macrolide antibiotics (e.g., fidaxomicin, azithrornycin, erythromycin, clarithromycin, roxithromycin, dirithromycin, telithrornycin), lincosarnide antibiotics (e.g., clindamycin, lincomycin), tetracycline antibiotics (e.g., demeclocycline, doxycycline, minocycline, oxytetracycline, tetracycline, tigecycline), streptograrnin antibiotics (e.g., pristinamycin, quiupristin/dallopristin), aminoglycoside antibiotics (e.g., amikacin, gentamicin, kanamycin, neomycin, netilmicin, paromomycin, streptomycin, tobramycin), cyclic lipopeptide antibiotics (e.g., daptomycin), lipoglycopeptide antibiotics (e.g., telavancin), glycopeptide antibiotics (e.g., vancomycin, teicoplanin), glycylcyciine antibiotics, oxazolidinone antibiotics (e.g., linezolid, cycloserine), tuberactinomycin antibiotics (e.g., viomycin, capreomycin), chloramphenicol, metronidazole, tinidazole, nitrofurantoin, or combinations thereof.

Other antimicrobials useful in the articles and methods invention include, without limitation, antifungal agents selected form amphotericin, butoconazole nitrate, clotrimazole, econazole nitrate, fluconazole, flucytosine, griseofulvin, itraconazole, ketoconazole, miconazole, natamycin, nystatin, sulconazole nitrate, terbinafine, terconazole, tioconazole, undecenoic acid, and their pharmaceutically acceptable salts. Articles including an antimicrobial can be useful for treating, or reducing the risk of, infection.

In some embodiments of any of the articles and methods provided herein the second agent can be an anti-proliferative. Antiproliferatives useful in the articles and methods of the invention include, without limitation, rapamycin, CCI-779, Everolimus, ABT-578, mechlorethamine, cyclophosphamide, iosfamide, melphalan, chlorambucil, uracil mustard, estramustine, mitomycin C, AZQ, thiotepa, busulfan, hepsulfam, carmustine, lomustine, semustine, streptozocin, dacarbazine, cisplatin, carboplatin, procarbazine, methotrexate, trimetrexate, fluouracil, floxuridine, cytara bine, fludarabine, capecitabine, azacitidine, thioguanine, mercaptopurine, allopurine, cladribine, gemcitabine, pentostatin, vinblastine, vincristine, etoposide, teniposide, topotecan, irinotecan, camptothecin, 9-aminocamptothecin, paclitaxel, docetaxel, daunorubicin, doxorubicin, dactinomycin, idarubincin, plicamycin, mitomycin, amsacrine, bleomycin, aminoglutethimide, anastrozole, finasteride, ketoconazole, tamoxifen, flutamide, leuprolide, goserelin, Gleevec™, leflunomide, SU5416, SU6668, PTK787 (Novartis), Iressa™ (AstraZeneca), Tarceva™, trastuzumab, Erbitux™, PKI166, GW2016, EKB-509, EKB-569, MDX-H210, 2C4, MDX-447, ABX-EGF, CI-1033, Avastin™, IMC-1C11, ZD4190, ZD6474, CEP-701, CEP-751, MLN518, PKC412, 13-cis-retinoic acid, isotretinoin, retinyl palmitate, 4-(hydroxycarbophenyl) retinamide, misonidazole, nitracrine, mitoxantrone, hydroxyurea, L-asparaginase, interferon alfa, AP23573, Cerivastatin, Troglitazone, CRx-026, DHA-paclitaxel, Taxoprexin, TPI-287, Sphingosine-based lipids, mitotane, and their pharmaceutically acceptable salts. Articles including an antiproliferative can be useful for treating any disease or condition associated with unwanted proliferation of cells.

In some embodiments of any of the articles and methods provided herein the second agent is a kinase inhibitor. Kinase inhibitors useful in the articles and methods invention include, without limitation, rlotinib (TARCEV A®), gefitinib (IRESSA®), cetuximab, sorafenib (NEXAVAR), dasatinib, ZD6474 (ZACTIMA), lapatinib (TYKERB), ST1571, imatinib (GLEEVEC), lestaurtinib (CEP-701), sunitinib maleate (SUTENT), panitumumab, EMD 72000, TheraClM hR3, EKB-569, 2C₄, AMG706, MP-412, XL647, XL 999, MLN518, PKC412, AMN107, AEE708, OSI-930, OSI-817, AG-013736, and their pharmaceutically acceptable salts. In some embodiments, the second agent is an anti-VEGF kinase inhibitor selected from 4-(4-bromo-2-fluoroanilino)-6-methoxy-7-(1-methylpiperidin-4-ylmethoxy)quinazoline (ZD6474), 4-(4-fluoro-2-methylindol-5-yloxy)-6-methoxy-7-(3-pyrrolidin-1-ylpropoxy)quinazoline (AZD2171), vatalanib (PTK787), semaxaminib (5U5416), and SUTENT® (sunitinib), and their pharmaceutically acceptable salts. Articles including a kinase inhibitor can be useful for treating any disease or condition associated with unwanted proliferation of cells and/or unwanted vascularization.

In an embodiment of any of the above articles and methods the second agent can be a prostaglandin selected from arbaprostil, alprostadil, beraprost, bimatoprost, carboprost, cloprostenol, dimoxaprost, dinoprost, enprostil, enisoprost, fluprostenol, fenprostalene, froxiprost, gemeprost, latanoprost, limaprost, meteneprost, mexiprostil, misoprostol, misoprost, misoprostol acid, nocloprost, ONO 373, ornoprostil, prostalene, PGE1, PGE2, PGF1, PGF2a, rioprostil, rosaprostol, remiprostol, sulprostone, tafluprost, trimoprostil, tiprostanide, travoprost, unoprostone, viprostol, viprostol, and their pharmaceutically acceptable salts. Articles including a prostaglandin can be useful for treating, or reducing the risk of, hypertension.

In some embodiments of any of the articles and methods provided herein the second agent is a steroid selected from anabolic steroids, androgenic steroids, progestin steroids, estrogen steroids, cancer treatment steroids, antibiotic steroids, glucocorticoid steroids, benign steroids, corticosteroids, anti-angiogenic steroids, intraocular pressure (IOP) lowering steroids, cholic acid-related bile acid steroids, steroid metabolites, cholesterol-derivatives, mineralocorticoid steroids, neurosteroids, pheromones, progestins, or other steroids, can be used in drug dimers. Examples of anabolic steroids include androisoxazole, androstenediol, bolandiol, bolasterone, clostebol, ethylestrenol, formyldienolone, 4-hydroxy-19-nortestosterone, methandriol, methenolone, methyltrienolone, nandrolone, norbolethone, oxymesterone, stenbolone, and trenbolone. Androgenic steroids are, for example, boldenone, fluoxymesterone, mestanolone, mesterolone, methandrostenolone, 17-methyltestosterone, 17-α-methyltestosterone 3-cyclopentyl enol ether, norethandrolone, normethandrone, oxandrolone, oxymesterone, oxymetholone, prasterone, stanlolone, stanozolol, testosterone, testosterone 17-chloral hemiacetal, testosterone proprionate, testosterone enanthate tiomesterone dehydroepiandrosterone (DHEA), androstenedione, androstenediol, androsterone, dihydrotestosterone (DHT), androstanolone, and derivatives thereof. Exemplary progestin steroids are norethisterone, norethisterone acetate, gestodene, levonorgestrel, allylestrenol, anagestone, desogestrel, dimethisterone, dydrogesterone, ethisterone, ethynodiol, ethynodiol diacetate, etonogestrel, gestodene, ethinylestradiol, haloprogesterone, 17-hydroxy-16-methylene-progesterone, 17 alpha-hydroxyprogesterone, lynestrenol, medroxyprogesterone, melengestrol, norethindrone, norethynodrel, norgesterone, gestonorone, norethisterone, norgestimate, norgestrel, levonorgestrel, norgestrienone, norvinisterone, pentagestrone, MENT (7-methyl-19-testosterone); norelgestromin, and trimigestone drospirenone, tibolone, megestrol, and derivatives thereof. Examples of estrogen steroid are estrogen, eguilenin, equilin, 17β-estradiol, estradiol benzoate, estriol, ethinyl estradiol, mestranol, moxestrol, mytatrienediol, quinestradiol, and quinestrol. Steroids used in cancer treatment are, for example, abiraterone, cyproterone acetate, dutasteride, enzalutamide, finasteride, and galeterone. Exemplary antibiotic steroid is fusidic acid. Glucocorticoids include, for example, medrysone, alclometasone, alclometasone dipropionate, amcinonide, beclometasone, beclomethasone dipropionate, betamethasone, betamethasone benzoate, betamethasone valerate, budesonide, ciclesonide, clobetasol, clobetasol butyrate, clobetasol propionate, clobetasone, clocortolone, loprednol, cortisol, cortisone, cortivazol, deflazacort, desonide, desoximetasone, desoxycortone, desoxymethasone, dexamethasone, diflorasone, diflorasone diacetate, diflucortolone, diflucortolone valerate, difluorocortolone, difluprednate, fluclorolone, fluclorolone acetonide, fludroxycortide, flumetasone, flumethasone, flumethasone pivalate, flunisolide, flunisolide, fluocinolone, fluocinolone acetonide, fluocinonide, fluocortin, fluocoritin butyl, fluocortolone, fluorocortisone, fluorometholone, fluperolone, fluprednidene, fluprednidene acetate, fluprednisolone, fluticasone, fluticasone propionate, formocortal, halcinonide, halometasone, hydrocortisone, hydrocortisone acetate, hydrocortisone aceponate, hydrocortisone buteprate, hydrocortisone butyrate, loteprednol, meprednisone, 6α-methylprednisolone, methylprednisolone, methylprednisolone acetate, methylprednisolone aceponate, mometasone, mometasone furoate, mometasone furoate monohydrate, paramethasone, prednicarbate, prednisolone, prednisone, prednylidene, rimexolone, tixocortol, triamcinolone, triamcinolone acetonide, and ulobetasol. Exemplary benign steroids are cholesterol, 11-deoxycortisol, 11-deoxycorticosterone, pregnenolone, cholic acid, chenodeoxycholic acid, ursodeoxycholic acid, obeticholic acid, tetrahydrocortisone, tetrahydrodeoxycortisol, tetrahydrocorticosterone, 5α-dihydrocorticosterone, and 5α-dihydropregesterone. Exemplary anti-angiogenic steroids or intraocular pressure (IOP) lowering steroids are anecortave acetate, anecortave, 11-epicortisol, 17α-hydroxyprogesterone, tetrahydrocortexolone, and tetrahydrocortisol. Exemplary cholic acid-related bile acid steroids are deoxycholic acid, apocholic acid, dehydrocholic acid, glycochenodeoxycholic acid, glycocholic acid, glycodeoxycholic acid, hyodeoxycholic acid, lithocholic acid, α-muricholic acid, β-muricholic acid, γ-muricholic acid, ω-muricholic acid, taurochenodeoxycholic acid, taurocholic acid, taurodeoxycholic acid, taurolithocholic acid, and tauroursodeoxycholic acid. Exemplary mineralocorticoid steroids are fludrocortisone and aldocortisone. Exemplary neurosteroids are alphaxalone, alphadolone, hydroxydione, minaxolone, tetrahydrodeoxycorticosterone, allopregnanolone, pregnanolone, ganoxolone, 3α-androstanediol, epipregnanolone, isopregnanolone, and 24(S)-hydroxycholesterol. Exemplary other steroids are flugestone, prebediolone, chlormadinone acetate, medrogestone, and segesterone acetate. Exemplary pheromones are androstadienol, androstadienone, androstenol, androstenone, estratetraenol, 5-dehydroprogesterone, 6-dehydro-retroprogesterone, allopregnanolone, and hydroxyprogesterone caproate. Exemplary steroid metabolites are tetrahydrotriamcinolone, cortienic acid, 11-dehydrocorticosterone, 11β-hydroxypregnenolone, ketoprogesterone, 17-hydroxypregnenolone, 17,21-dihydroxypregnenolone, 18-hydroxycorticosterone, deoxycortisone, 21-hydroxypregnenolone, and progesterone. Exemplary progestins are allopregnone-3α,20α-diol, allopregnone-3β,20β-diol, allopregnane-3β,21-diol-11,20-dione, allopregnane-3β,17α-diol-20-one, 3,20-allopregnanedione,3β,11β,17α, 20β,21-pentol, allopregnane-3β,17α, 20β,21-tetrol, allopregnane-3α,11β,17α, 21-tetrol-20-one, allopregnane-3β,11β,17α, 21-tetrol-20-one, allopregnane-3β,17α,20β-triol, allopregnane-3β,17α, 21-triol-11,20-dione, allopregnane-3β,11β,21-triol-20-one, allopregnane-3β,17α, 21-triol-20-one, allopregnane-3α-ol-20-one, allopregnane-3β-ol-20-one, pregnanediol, 3,20-pregnanedione, 4-pregnene-20,21-diol-3,11-dione, 4-pregnene-11β,17α, 20β,21-tetrol-3-one, 4-pregnene-17α, 20β,21-triol-3,11-dione, 4-pregnene-17α,20β,21-triol-3-one, and pregnenolone.

In some embodiments of any of the articles and methods provided herein the second agent is a non-steroidal anti-inflammatory drug (NSAID) selected from acetylsalicylic acid, celecoxib, rofecoxib, valdecoxib, diclofenac, diflunisal, etodolac, ibuprofen, flurbiprofen, indomethacin, ketoprofen, ketorolac, nabumetone, naproxen, oxaprozin, piroxicam, sulindac, tolmetin, meclofenamate, mefenamic acid, and meloxicam.

In some embodiments of any of the articles and methods provided herein the second agent is an immunomodulatory agent selected from cyclosporin, azathioprine, methotrexate, mycophenolate, pegfilgrastim)(Neulasta®, lenalidomide (CC-5013, Revlimid®), thalidomide) (Thalomid®, actimid (CC4047), temsirolimus, ridaforolimus, everolimus, rapamycin)(Sirolimus®; simapimod, and emsirolimus.

In some embodiments of any of the articles and methods provided herein the second agent is a statin selected from atorvastatin)(Lipitor®, fluvastatin (Lescol®, Lescol XL®), lovastatin (Mevacor®, Altoprev®), pravastatin (Pravachol®), rosuvastatin)(Crestor®, simvastatin (Zoco®r), pitavastatin (Livalo®), and their pharmaceutically acceptable salts. Articles including a statin can be useful for treating, or reducing the risk of, high cholesterol.

In some embodiments of any of the articles and methods provided herein the second agent is an antihypertensive selected from thiazide diuretics (e.g., chlorothiazide), calcium channel blockers, ACE inhibitors, angiotensin II receptor antagonists, and beta blockers. The antihypertensive can be a calcium channel blocker selected from amlodipine, aranidipine, azelnidipine, barnidipine, benidipine, cilnidipine, clevidipine, efonidipine, felodipine, isradipine, lacidipine, lercanidipine, manidipine, nicardipine, nifedipine, nilvadipine, nimodipine, nisoldipine, nitrendipine, nitrepin, pranidipine, fendiline, gallopamil, verapamil, and their pharmaceutically acceptable salts. The antihypertensive can be an ACE inhibitor selected from benazepril, zofenopril, perindopril, trandolapril, captopril, enalapril, lisinopril, ramipril, and their pharmaceutically acceptable salts. The antihypertensive can be an angiotensin II receptor antagonist selected from candesartan, irbesartan, losartan, and their pharmaceutically acceptable salts. The antihypertensive can be a beta blocker selected from propranolol, bucindolol, carteolol, carvedilol, labetalol, nadolol, oxprenolol, penbutolol, pindolol, sotalol, timolol, acebutolol, atenolol, betaxolol, bisoprolol, celiprolol, metoprolol, nebivolol, esmolol, butaxamine, and their pharmaceutically acceptable salts. Articles including an antihypertensive can be useful for treating, or reducing the risk of, high blood pressure.

In some embodiments of any of the articles and methods provided herein the second agent is a vasodilator selected from prostaglandins (e.g., prostaglandin D2 or prostaglandin E2), histamine, L-arginine, niacin, sildenafil, vardenafil, tadalafil, and their pharmaceutically acceptable salts. Articles including a vasodilator can be useful for treating, or reducing the risk of, high blood pressure, angina, congestive heart failure, or erectile dysfunction.

In some embodiments of any of the articles and methods provided herein the second agent is an antioxidant selected from resveratrol, beta-carotene, glutathione, vitamin E, vitamin C, butylated hydroxytoluene, butylated hydroxyanisole, tertiary-butylhydroquinone, propyl gallate, ethoxyquin, and their pharmaceutically acceptable salts. Articles including an antioxidant can be useful for treating, or reducing the risk of, oxidative stress.

In some embodiments of any of the articles and methods provided herein the second agent is a neuroprotective agent selected from NMDA receptor stimulants (e.g., selegiline, nicotine, caffeine), and glutamate antagonists (e.g., estrogen, ginsenoside, progesterone, simvastatin, memantine), and their pharmaceutically acceptable salts. Articles including a neuroprotective agent can be useful for treating, or reducing the risk of, nerve damage or CNS disorders.

### Articles

The articles of the invention can be in the form of fibers, fiber meshes, woven fabrics, non-woven fabrics, films, pellets, cylinders, microparticles, nanoparticles, or other shaped articles. In some embodiments, the pharmaceutical composition of the invention has a non-circular shape that affects, e.g., increases, the surface area (e.g., extruded through star-shaped dye). Suitable articles for use with this invention can be small regularly or irregularly shaped particles, which can be solid, porous, or hollow (e.g., a cylindrical tube). The articles of the invention can also be a coating on the surface of a device, such as an implantable medical device.

Different forms of articles of the invention (e.g., fibers, fiber meshes, woven fabrics, non-woven fabrics, films, pellets, cylinders, microparticles, nanoparticles, or other shaped articles) can have the advantages of providing a controllable surface area, being easily injected, not requiring removal after completion of drug release, and allow for tailoring drug release rates required for a given indication. When used as an injectable drug delivery device, drug release rate and interaction with cells are strongly dependent on the size distribution of the pharmaceutical composition form.

Steroid Dimer as a Binding Agent

In some embodiments, the articles provided herein are formed by the use of the steroid dimer of formula (A-I) as a binder for the second agent, wherein the second agent is distributed heterogeneously within the admixture. Such articles can be formed from an admixture of: (i) from about 5% to 45% (w/w) (e.g., 7.5±2.5%, 10±2.5%, 12.5±2.5%, 15±2.5%, 20±5%, 25±5%, 30±5%, 35±5%, or 40±5% (w/w)) of a compound of formula (A-I):

D1-L-D2 (A-I),

or a pharmaceutically acceptable salt thereof, wherein each of D1 and D2 is, independently, a radical formed from a steroid; and L is a linker covalently linking D1 to D2; and (ii) from 55% to 95% (w/w) (e.g., 60±5%, 65±5%, 70±5%, 75±5%, 80±5%, 85±5%, or 90±5% (w/w)) of a second agent, wherein the second agent is a therapeutic drug or more than 1 therapeutic drug. The article can be free of controlled release polymer. In one embodiment, the (w/w) ratio of the compound of formula (A-I) to the second agent is from 1:2 to 1:20 (e.g., from 1:2 to 1:4, 1:4 to 1:20, or 1:8 to 1:20). In this scenario, the steroid dimer acts predominantly as a binder to form a depot, but does not necessarily or minimally alters the release profile of the second agent.

### Processing Methods

In some embodiments, articles or pharmaceutical compositions provided herein are formed using methods, for example, heat processing or solvent processing of the drug dimer of formula (I). Heat processing can include heat molding, injection molding, extrusion, 3D printing, melt electrospinning, fiber spinning, fiber extrusion, and/or blow molding. Solvent processing may include coating, micro printing, emulsion processing, dot printing, micropatterning, drop and drag, fiber spinning, solvent blow molding, electrospraying, and electrospinning.

Electrospraying Method

In some embodiments, the pharmaceutical compositions provided herein are dissolved in a solvent (e.g., acetone) at concentrations ranging from, e.g., 10-30% w/v, and are electrosprayed to form micro- and/or nanoparticles. The solutions can be loaded into a syringe and can be injected at a particular rate, e.g., 0.5 mL/h, onto a stationary collection plate. Between the needle and collecting surface, a potential difference of, e.g., 18 kV, can be maintained. In some embodiments, a concentration of 10% w/v is used to obtain nanoparticles. In other embodiments, a concentration of 30% w/v is used to obtain microparticles.

Fiber Spinning Methods

In some embodiments, the pharmaceutical compositions provided herein, e.g., fibrous meshes with aligned and unaligned morphologies are prepared by electrospinning. The pharmaceutical compositions provided herein are dissolved in a solvent (e.g., THF, or 1:1 ratio of DCM/THF). The solutions may be injected from a syringe at a particular rate, e.g., 0.5 mL/h, onto a cylindrical mandrel rotating at a particular rotational speed, e.g., 1150 rpm, to obtain aligned fibers, or onto a stationary collector surface to obtain unaligned fibers. A potential difference (e.g., 18 kV or 17 kV) can be maintained between the needle and collecting surface for aligned and random fibers.

In other embodiments, fibers are prepared either from the melt at elevated temperatures, the glassy state intermediate, or from solution by dissolving the pharmaceutical compositions provided herein in a solvent (e.g., DCM, THF, or chloroform). As used herein, melt spinning describes heat processing from the melt state, heat spinning describes heat processing from the glassy state, and wet, dry, and gel spinning describe solution processing.

The viscous melt, intermediate, or solution can be fed through a spinneret and fibers may be formed upon cooling (melt or heat spinning) or following solvent evaporation with warm air as the compound exits the spinneret (dry spinning). Wet spinning and gel spinning, performed according to methods known in the art, may also be used to produce the fibers provided herein. Heat spinning describes a process that is essentially the same as the melt spinning process, but performed with the glassy state intermediate and heated above the glass transition temperature (Tg) to get the viscous fluid to extrude/spin instead of the melt. Alternatively, tweezers may be dipped into melted material or concentrated solutions and retracted slowly in order to pull fibers. The rate of pulling and distance pulled may be varied to yield fibers and columnar structures of different thickness.

Emulsion Method

In some embodiments, micro-particles or nano-particles made from the pharmaceutical composition can be formed using an emulsion process. The pharmaceutical composition may be dissolved in an organic solvent (e.g. DCM, THF, etc.) and a surfactant (e.g. SDS, PVA, etc.) may be added to the solution/mixture at a low percentage (e.g. 1%). The resulting mixture may be stirred for the appropriate time at room temperature to form an emulsion. The emulsion may be subsequently added to Milli-Q water under stirring for an appropriate time (e.g. 1 h) to remove residual solvent. The resulting micro- or nano-particles may be collected by centrifugation and dried to obtain the desired form.

Extrusion Method

In some embodiments, injectable cylinders made from the pharmaceutical composition may be formed by heat extrusion. The pharmaceutical composition may be loaded into a hot melt extruder, heated to a temperature above the melting point (for crystalline compositions) or glass transition temperature (for pre-melted or amorphous compositions), and extruded using a light compressive force to push the material through the nozzle and a light tensile force to pull the material out of the extruder. The extrudate may be cut to the desired length for appropriate drug dosing for the indication of interest.

Bead Sizing and Milling

In some embodiments, a milling process may be used to reduce the size of an article provided herein to form sized particles, e.g., beads, in the micrometer (microbeads) to nanometer size range (nanobeads). The milling process may be performed using a mill or other suitable apparatus. Dry and wet milling processes such as jet milling, cryo-milling, ball milling, media milling, sonication, and homogenization are known and can be used in methods described herein. Generally, in a wet milling process, a suspension of the material to be used as the core is agitated with or without excipients to reduce particle size. Dry milling is a process wherein the material to be used as the article core is mixed with milling media with or without excipients to reduce particle size. In a cyro-milling process, a suspension of the material to be used as the core is mixed with milling media with or without excipients under cooled temperatures. In some embodiments, subsequent heating of the milled microparticle above the Tg is needed to achieve a spherical shape, or particles with non-spherical shapes can be used as milled.

Low Temperature Processing Using Intermediate Glassy State Articles

In certain embodiments, the prodrug dimer or second agent has a limited window (e.g., short timeframe of seconds to minutes) of thermal stability, whereby the purity of the dimer or second agent is minimally affected at elevated temperatures. In some embodiments, it is beneficial to make an intermediate glassy state form of the dimer, or second agent, or both (e.g., film, pellet, micro-particles, or other shaped article). This can be accomplished by heat or solvent processing to remove or reduce the crystallinity of the material to form a glassy state composition. The glassy state composition is subsequently heat processed at a lower temperature (e.g., processing just above the glass transition temperature (Tg), and below the melt temperature (Tm)). This can provide a longer timeframe for heat processing the glassy state material into the final shaped article, while reducing the impact of processing conditions on the purity of the prodrug dimer and/or second agent in the article.

Layered and Unlayered Articles

The articles of the invention can be homogenous mixtures of the steroid dimer and the second agent (e.g., for the sustained release of the second agent). In other embodiments, the articles include a first portion containing predominantly the steroid drug dimer and a second portion containing predominantly the second agent. For example, a core formed from the steroid dimer can be coated with the second agent to produce an article formulated for immediate release of the second agent. Alternatively, a core formed from the second agent can be coated with the steroid dimer to produce an article formulated for delayed release of the second agent.

Layering can occur layer by layer for example by depositing one layer and then the next layer on top and repeating this as many times as necessary to get the desired article. This can be done, for example, by different coating methods (e.g. drop coating, spray coating, dip coating, etc.), electrospinning, electrospraying, or other methods through solvent or heat processing. This can also be done in one process, for example, by co-extruding a core layer with additional layers around the core layer.

Exemplary processing details are provided in the Examples.

Drug Delivery

In some embodiments, articles and pharmaceutical compositions provided herein provide optimal delivery of drug (e.g., D1, D2, and/or the second agent) release from an article in a controlled manner, for example, by surface erosion. In some embodiments, the surface erosion mechanism of second agent release may allow the shaped article to maintain its physical form, while gradually decreasing in size as the surface erodes (e.g., like a bar of soap). The drug can be controlled to be delivered over a desired period of time. A slower and steadier rate of delivery (e.g., release of less than 10% of D1 or D2 (as a percentage of the total drug, D1, D2, the at least one therapeutic agent, or a combination thereof present in the article or pharmaceutical composition in prodrug form) at 37° C. in 100% bovine serum over 5 days) may in turn result in a reduction in the frequency in the administration of the pharmaceutical composition or article, and improve the safety profile of the drug or combination of drugs. Alternatively, articles can be designed for immediate release or delayed release for use in situations where prolonged drug release (even at low doses) could result in unwanted side effects (e.g. cataract formation in the eye with prolonged steroids) or undesired consequences (e.g. antibiotic resistance).

In some embodiments, the rate of release of the second agent depends on many factors including, for example, the drug composition of the drug dimer (e.g., steroid dimer). Drug release rate from the article of the drug dimer (e.g., steroid dimer) can be modulated by the cleavage of drug-linker bond through hydrolysis or enzymatic degradation. In some embodiments, the linker can affect drug release rate. This in turn can modulate the release of the second agent trapped by the steroid dimer. In some embodiments, the drug release rate is controlled by a functional group on the drug to conjugate through to the linker, for example, a primary vs. a secondary steroid hydroxyl group. The rate of release of a given drug from a drug dimer may also depend on the quantity of the loaded drug dimer as a percent of the final drug dimer formulation, e.g., by using a pharmaceutical excipient or a second steroid drug (e.g., active or benign) as a homodimer mixture, or within the same molecule as a heterodimer that acts as a bulking agent. Another factor that can affect the release rate of a drug from, for example a microbead, is the microbead size. In some embodiments, drug release is tailored based on the solubility of drug dimer (e.g., through selection of appropriate drug and/or linker) that will influence the rate of surface erosion (e.g., dissolution/degradation) from the article. In other embodiments, drug release is affected by changes in surface area of the formulation, e.g., by changing the diameter of the microbeads. By adjusting the vide supra factors, dissolution, degradation, diffusion, and controlled release may be varied over wide ranges. For example, release may be designed to be initiated over minutes to hours, and may extend over the course of days, weeks, months, or years.

Uses and Pharmaceutical Compositions

In some embodiments, the articles of the invention are used as a drug delivery device (or, e.g., a drug depot) with a minimal need for additives. This may achieve a local, sustained release and a local biological effect, while minimizing a systemic response. In some embodiments, when present, the additives are in small amounts and do not affect the physical or bulk properties. In some embodiments, when present, the additives do not alter the drug release properties from the pharmaceutical composition but rather act to improve processing of the prodrug dimer into the shaped article. In some embodiments, the pharmaceutical compositions contain additives such as a plasticizer (e.g., to reduce thermal transition temperatures), an antioxidant (e.g., to increase stability during heat processing), a binder (e.g., to add flexibility to the fibers), a bulking agent (e.g., to reduce total drug content), a lubricant, a radio-opaque agent, or mixtures thereof. The additives may be present at 30% (w/w), e.g., 20% (w/w), 10% (w/w), 7% (w/w), 5% (w/w), 3% (w/w), 1% (w/w), 0.5% (w/w), or 0.1% (w/w). Examples of plasticizers are polyols, e.g., glycerol, ethylene glycol, diethylene glycol, triethylene glycol, tetraethylene glycol, polyethylene glycol, propylene glycol, triacetin, sorbitol, mannitol, xylitol, fatty acids, monosaccharides (e.g., glucose, mannose, fructose, sucrose), ethanolamine, urea, triethanolamine, vegetable oils, lecithin, or waxes. Exemplary antioxidants are glutathione, ascorbic acid, cysteine, or tocopherol. The binders and bulking agents can be, e.g., polyvvinylpyrrolidone (PVP), starch paste, pregelatinized starch, hydroxypropyl methyl cellulose (HPMC), carboxymethyl cellulose (CMC), or polyethylene glycol (PEG) 6000.

Methods involving treating a subject may include preventing a disease, disorder or condition from occurring in the subject which may be predisposed to the disease, disorder and/or condition but has not yet been diagnosed as having it; inhibiting the disease, disorder or condition, e.g., impeding its progress; and relieving the disease, disorder, or condition, e.g., causing regression of the disease, disorder and/or condition. Treating the disease or condition includes ameliorating at least one symptom of the particular disease or condition, even if the underlying pathophysiology is not affected (e.g., such treating the pain of a subject by administration of an agent even though such agent does not treat the cause of the pain).

Pharmaceutical compositions containing the drug dimers described herein may be administered to a subject via any route known in the art. These include, but are not limited to, oral, sublingual, nasal, intradermal, subcutaneous, intramuscular, rectal, vaginal, intravenous, intraarterial, intracisternally, intraperitoneal, intravitreal, periocular, topical (as by powders, creams, ointments, or drops), buccal and inhalational administration. Desirably, the articles of the invention are administered parenterally as injections (intravenous, intramuscular, or subcutaneous), or locally as injections (intraocularly or into a joint space). The formulations are admixed under sterile conditions with a pharmaceutically acceptable carrier and any needed preservatives or buffers as may be required.

In some embodiments, an article provided herein is administered to a subject to deliver a therapeutically effective amount of the second agent as part of prophylactic or therapeutic treatment. In general, an effective amount of the second agent refers to the amount necessary to elicit the desired biological response alone or in combination with the hydrolyzed steroid dimer. In some embodiments, the concentration of the pharmaceutical agent (e.g., in the article) provides an article with certain physical and/or biological properties, such as, for example, a certain absorption profile, inactivation profile, excretion rate, delivery rate, biological endpoint, delivery agent, or target tissue of any component of the article. It is to be noted that dosage values may also vary with the severity of the condition to be alleviated. It is to be further understood that for any particular subject, specific dosage regimens should be adjusted over time according to the individual need and the professional judgment of the person administering or supervising the administration of the compositions. In some embodiments, dosing is determined using techniques known to one skilled in the art.

In some embodiments, the concentration and/or amount of any pharmaceutical agent to be administered to a subject is determined by one of ordinary skill in the art. Known methods are also available to assay local tissue concentrations, diffusion rates from drug dimers and local blood flow before and after administration of the therapeutic formulation.

Sterilization of Formulations

In certain aspects, an article is sterile before or upon administration to a subject. In some embodiments, a sterile formulation is essentially free of pathogenic microorganisms, such as bacteria, microbes, fungi, viruses, spores, yeasts, molds, and others generally associated with infections. In some embodiments, articles provided herein are subjected to an aseptic process and/or other sterilization process. An aseptic process can involve sterilizing the components of a formulation, final formulation, and/or container closure of a drug product through a process such as heat, gamma irradiation, ethylene oxide, or filtration and then combining in a sterile environment. In some cases, an aseptic process is preferred. In other embodiments, terminal sterilization is preferred.

### Treatment Methods

In certain instances, the articles and pharmaceutical compositions provided herein are implanted in an individual (e.g., the implant employed in the fields of ophthalmology, oncology, laryngology, endocrinology and metabolic diseases, rheumatology, urology, neurology, cardiology, dental medicine, dermatology, otology, post-surgical medicine, orthopedics, pain management, and/or gynecology).

In some embodiments, a compound provided herein is selected for a particular property, such as, for example, a corticosteroid dimer for treating inflammatory diseases or conditions; an antibiotic steroid dimer for treating an infection; an anticancer steroid dimer for treating a proliferative disorder; or a benign steroid for controlling the release of the second agent without producing any significant local effect following hydrolysis of the prodrug steroid dimer.

Ophthalmic Uses

In certain embodiments, the articles of the invention may be used prevent, treat or manage diseases or conditions at the back of the eye, such as at the retina, macula, choroid, sclera and/or uvea.

In some embodiments, provided herein is an injectable drug delivery device (e.g., comprising an article provided herein) for ophthalmology (e.g., intravitreal injection, coating on a minimally invasive glaucoma surgery (MIGS) devices, or implant in blebs). In certain embodiments, (e.g., during an intravitreal injection) a composition (e.g., an article, a coating, a pharmaceutical composition) provided herein is placed (e.g., directly) into the space in the back of the eye (e.g., called the vitreous cavity, which is filled with a jelly-like fluid called the vitreous humor gel). Intravitreal injections may be used to treat retinal diseases such as diabetic retinopathy, macular degeneration, macular edema, uveitis, and retinal vein occlusion.

In certain embodiments, an article provided herein may be used to treat, prevent, or manage an ocular condition, e.g., a disease, ailment, or condition that affects or involves the eye or one or more of the parts or regions of the eye. In some embodiments, an article provided herein is used to treat, prevent, or manage an ocular condition at the front of the eye of a subject. Non-limiting examples of a front of the eye ocular condition includes, but is not limited to, a disease, ailment or condition, such as for example, post-surgical inflammation; uveitis; infections; aphakia; pseudophakia; astigmatism; blepharospasm; cataract; conjunctival diseases; conjunctivitis; corneal diseases; corneal ulcer; dry eye syndromes; eyelid diseases; lacrimal apparatus diseases; lacrimal duct obstruction; myopia; presbyopia; pupil disorders; corneal neovascularization; refractive disorders and strabismus. In some embodiments, an article provided herein used to treat, prevent, or manage an ocular condition at the back of the eye of a subject. Non-limiting examples of a posterior ocular condition include, but are not limited to, a disease, ailment, or condition, such as intraocular melanoma; acute macular neuroretinopathy; Behcet's disease; choroidal neovascularization; uveitis; diabetic uveitis; histoplasmosis; infections, such as fungal or viral-caused infections; macular degeneration, such as acute macular degeneration, non-exudative age related macular degeneration and exudative age related macular degeneration; edema, such as macular edema (e.g., cystoid macular edema (CME) and diabetic macular edema (DME)); multifocal choroiditis; ocular trauma which affects a posterior ocular site or location; ocular tumors; retinal disorders, such as central retinal vein occlusion, diabetic retinopathy (including proliferative diabetic retinopathy), proliferative vitreoretinopathy (PVR), retinal arterial occlusive disease, retinal detachment, uveitic retinal disease; sympathetic opthalmia; Vogt Koyanagi-Harada (VKH) syndrome; uveal diffusion; a posterior ocular condition caused by or influenced by an ocular laser treatment; posterior ocular conditions caused by or influenced by a photodynamic therapy, photocoagulation, radiation retinopathy, epiretinal membrane disorders, branch retinal vein occlusion, anterior ischemic optic neuropathy, non-retinopathy diabetic retinal dysfunction, retinitis pigmentosa, retinoblastoma, and glaucoma. In some embodiments, provided herein is an article used to treat, prevent, or manage dry eye in a subject. In some embodiments, an article provided herein is used to treat, prevent, or manage inflammation in the eye of a subject (e.g., where the drug dimer is formed from one or more corticosteroids). In some embodiments, inflammation is associated with a variety of ocular disorders. In some embodiments, ophthalmic surgical procedures, including cataract surgery, produce an inflammatory response in an individual. In some embodiments, a pharmaceutical agent or article delivered into the eye (e.g., using the articles and/or methods provided herein) may be a corticosteroid. In certain embodiments, the pharmaceutical agent includes one or more of hydrocortisone, cortisone, tixocortol, prednisolone, methylprednisolone, prednisone, triamcinolone acetonide, mometasone, amcinonide, budesonide, desonide, fluocinonide, fluocinolone, halcinonide, betamethasone, dexamethasone, fluocortolone, hydrocortisone, aclometasone, prednicarbate, clobetasone, clobetasol, fluprednidene, glucocorticoid, mineralocorticoid, aldosterone, deoxycorticosterone, fludrocortisone, halobetasol, diflorasone, desoximetasone, fluticasone, flurandrenolide, alclometasone, diflucortolone, flunisolide, and beclomethasone. In some embodiments, an article provided herein is used as an adjunctive therapy to reduce, by way of non-limiting examples, inflammation and fibrosis (e.g., associated with a device described herein (e.g., minimally invasive glaucoma surgery (MIGS) devices)). In some embodiments, an article provided herein is for treating, preventing, or managing age-related macular degeneration (AMD) in a subject. The second agent can be a prostaglandin (e.g., for treating, managing, and/or reducing (e.g., the risk of) intraocular pressure.

In some embodiments, the second agent is a glaucoma therapeutic agent (e.g., a therapeutic agent that has activity for treating or reducing the symptoms of glaucoma). In some embodiments, the second agent (e.g., the at least one therapeutic agent) is selected from the group consisting of a prostaglandin (e.g., latanoprost, bimatoprost, travoprost, tafluprost, sepetaprost, omidenepag isopropyl, and latanoprostene bunod), a beta-blocker (e.g., timolol (e.g., maleate, hemihydrate) and betaxolol), a rho kinase inhibitor (e.g., fasudil and netarsudil), an alpha-adrenergic agonist (e.g., brimonidine and apraclonidine), a carbonic anhydrase inhibitor (dorzolamide and brinzolamide), an adrenergic agonist (e.g., epinephrine) and a cholinergic agent (e.g., pilocarpine). In some embodiments the therapeutic agent is an anti-inflammatory agent and/or an intraocular pressure (IOP) lowering agent.

Osteoarthritis Treatment

In some embodiments, provided herein are article for treating osteoarthritis (OA). By way of a non-limiting example, for OA of the knee, intraarticular (IA) injection (e.g., of steroids) may be preferred (e.g., as the last non-operative modality) when other conservative treatment modalities are ineffective. Steroids may be used to reduce inflammation in tendons and ligaments in osteoarthritic joints. IA steroid injections may provide short term reduction in OA pain and can be considered as an adjunct to core treatment for the relief of moderate to severe pain in people with OA. Exemplary steroids used in the treatment of OA are betamethasone, methylprednisolone, dexamethasone, and triamcinolone. In some embodiments, provided herein are articles (e.g., provided herein and which are injected into a knee joint) for treating OA. In some embodiments, microspheres provided herein (e.g., comprising a steroid dimer and at least one therapeutic agent) are used for treating OA (e.g., upon injection into a knee joint). The second agent can be an analgesic or opioid useful for treating, or reducing the risk of, joint pain.

Surgical or Post-Surgical Care

Provided in some embodiments is an article used for treating a condition, a side-effect, or a complication of a procedure performed on an individual, the method comprising implanting an article provided herein into the individual.

Provided in some embodiments is an article used for preventing a condition, a side-effect, or a complication of a procedure performed on an individual, the method comprising implanting an article provided herein into the individual.

In some embodiments, the article is administered to the individual in need thereof after the procedure performed on the individual. In some embodiments, the article is administered to the individual in need thereof before the procedure performed on the individual. In some embodiments, the article is administered to the individual in need thereof during the procedure performed on the individual.

In some embodiments, the procedure is radiation. In some embodiments, the procedure is radiation therapy. In some embodiments, the second agent is an anti-proliferative agent. In some embodiments, the anti-proliferative agent reducing the risk of cancer (e.g., by inhibiting excessive cellular proliferation after surgery).

In some embodiments, the procedure is an emergency procedure. In some embodiments, the procedure is a planned procedure. In some embodiments, the procedure is a standard procedure.

Surgical Procedures

In some embodiments, the articles of the invention are used in conjunction with a surgical procedure. For example, an article can be implanted at a surgical site to reduce the risk of infection, inflammation, or the recurrence of disease (such as a cancer) treated by the surgical procedure. The second agent can be an antimicrobial useful for treating, or reducing the risk of, infection.

In some embodiments, the surgical procedure is an emergency surgical procedure. In some embodiments, the surgical procedure is a standard surgical procedure.

### EXAMPLES

The following examples are put forth to provide those of ordinary skill in the art with a description of how the compositions and methods described herein may be used, made, and evaluated, and are intended to be purely exemplary of the invention and are not intended to limit the scope of what the inventors regard as their invention.

### Example 1: Compound 1 (dexamethasone-triethylene glycol-dexamethasone; Dex-TEG-Dex) Can be Synthesized and Formed into Pellets and Films in the Glassy State that Provide Sustained Release of Dexamethasone

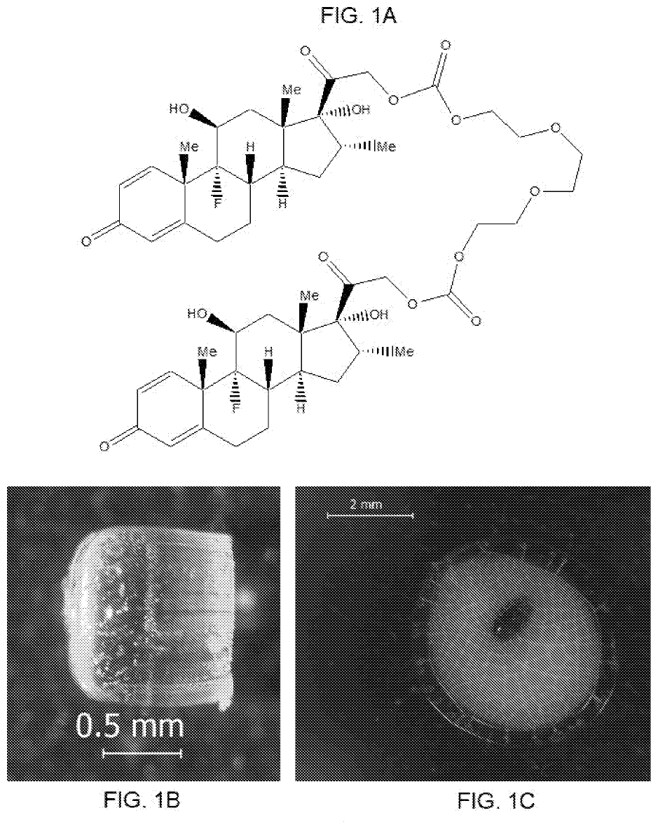

Dexamethasone (1 mol equivalent) was suspended in dichloromethane on an ice bath and triethylamine (2 mol equivalent) and triethylene glycol bis(chloroformate) (0.6 mol equivalent) were added to the mixture. The ice bath was allowed to warm to room temperature and the reaction was stirred overnight. The solvent was removed and the solid residue was purified by column chromatography. Product was recrystallized from acetonitrile twice to give Compound 1 (FIG. 1A) as an off-white crystalline solid.

Compound 1: HPLC (mobile phase: H₂O/TFA and MeCN/TFA) 31.7 min; Elemental analysis: Anal. Calcd for C₅₂H₆₈F₂O₁₆: C, 63.27; H, 6.94; N, 0.00; Cl, 0.00 Found: C, 62.62; H, 6.84; N, <0.50; Cl<100 ppm. ¹H NMR (400 MHz, DMSO-d₆) δ (ppm) 0.80 (d, J=7 Hz, 6H, 2× C₁₆α-CH₃); 0.90 (s, 6H, 2× C₁₈—CH₃); 1.08 (m, 2H, 2× C₁₆—H); 1.35 (m, 2H, 2× C₁₄—H); 1.49 (s, 6H, 2×C₁₉—CH₃); 1.54 (q, J=13 Hz, 2H, 2× C₁₃—H); 1.64 (q, J=11 Hz, 2H, 2× C₁₅—CH₂); 1.77 (m, 2H, 2×C₁₅—CH₂); 2.15 (m, 4H, 2× C₆—CH₂); 2.32 (m, 4H, 2× C₇—CH₂); 2.62 (m, 2H, 2× C₁₂—CH₂); 2.89 (m, 2H, 2× C₁₂—CH₂); 3.57 (s, 4H, 2×TEG OCH₂); 3.65 (m, 4H, 2×TEG OCH₂); 4.15 (m, 2H, 2×OCH); 4.22 (m, 4H, 2×TEG OCH₂); 4.79 (d, 2H, AB, J=18.5 Hz, 2H, C₂₁—CH₂O—); 5.09 (d, 2H, AB, J=18.5 Hz, 2H, C₂₁—CH₂O—); 5.18 (s, 2H, C₁₇—OH); 5.40 (d, 2H, J=4.5 Hz, C₁₁-0H); 6.01 (d, 2H, J=1.9 Hz, 2× alkene C₄—CH); 6.23 (dd, 2H, J=10.1 and 1.9 Hz, CH, 2× alkene C₂—CH); 7.29 (d, 2H, C₁—CH 2×alkene CH, 10.1 Hz, 2H). MS (ESI+) m/z: [M+H]+Calcd for C₅₂H₆₉F₂O₁₆987.46; Found 987.46.

Compound 1 was formed into pellets (FIG. 1B) in the glassy state by heat molding or into thin films (FIG. 1C) in the glassy state by solvent casting from the solution state. Briefly, crystalline powder was melted at 185° C. and pellets were formed from 1 mm×1 mm cylindrical molds. Alternatively, crystalline powder was also dissolved in acetone, was solvent cast onto Dacron, and the solvent was evaporated to form the film.

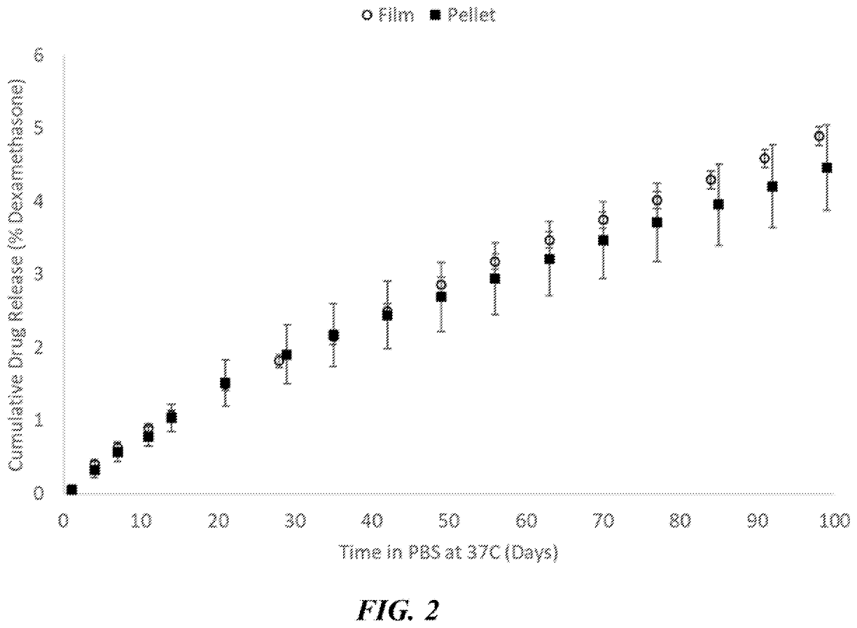

Heat-molded pellets from Compound 1 (^{˜}1 mm×1 mm) were placed in 20 mL glass vials and 2 mL of the release buffer phosphate buffered saline (PBS) was added. Samples were incubated at 37° C. on a shaker rotating at 115 rpm. After 1 day, 3 days, 7 days, and subsequently in alternating 3- and 4-day intervals (e.g., 1, 3, 7, 10, 14 days etc.), release buffer was analyzed by high performance liquid chromatography (HPLC) to quantify drug products. Cumulative drug release was calculated and plotted as a percentage of the total drug in each pellet released over time (FIG. 2). FIG. 2 show a zero-order (e.g., near zero order) release of an article of Compound 1 for at least 100 days.

### Example 2: A Sustained Release Formulation Consisting of Compound 1 (Dex-TEG-Dex) and Paclitaxel can be Formed into Multiple Physical Forms Through Different Processing Techniques

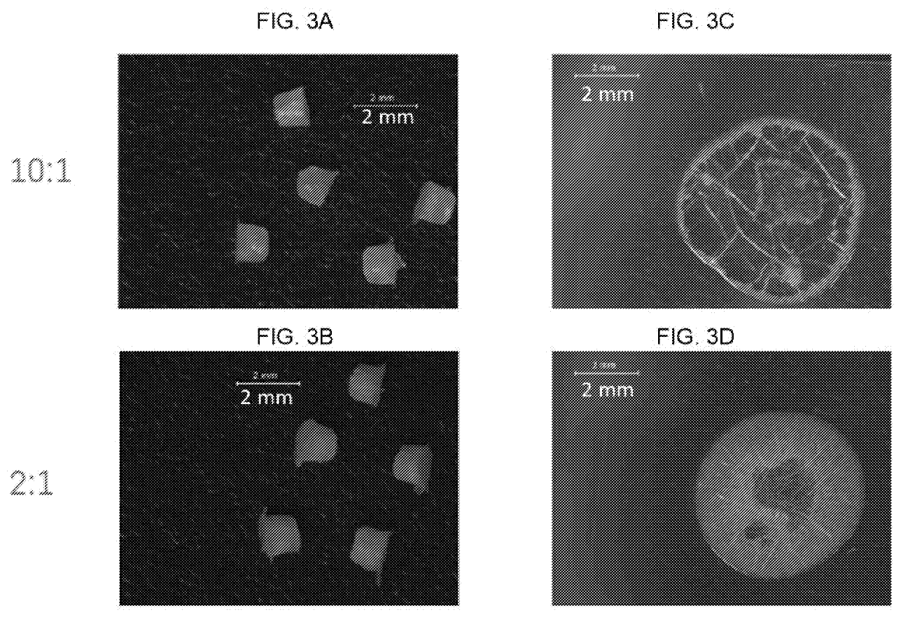

Compound 1 and paclitaxel were weighed in either 10:1 or 2:1 (mass:mass) ratios into a ceramic mortar and ground until visually homogeneous. The combined crystalline powder was melted at 185° C. and pellets were formed from 1 mm×1 mm cylindrical molds to produce pellets of roughly 1 mg total mass (FIG. 3A and FIG. 3B for the two ratios respectively). The heat molded pellets formed resulted in compound 1 being in the glassy state and paclitaxel in the crystalline state.

Films of the same ratios were prepared on Dacron coupons using a solvent casting (drop coating) technique. Compound 1 was dissolved in acetone to a concentration of 100 mg/mL and this solution was subsequently used to solubilize paclitaxel at 10 and 50 mg/mL concentrations to yield the same 10:1 and 2:1 ratio used during pellet formation. Droplets (10 μL) were deposited in the middle of 1 cm×1 cm Dacron coupons and were allowed to dry under air flow in a fume hood overnight to yield films of ^{˜}1 mg material analogous to pellet loading. Optical imaging of the resultant films coincided with a visibly-elevated amount of crystallinity with higher amounts of paclitaxel to the otherwise transparent (glassy state) compound 1 films (FIG. 3C and FIG. 3D for the 10:1 and 2:1 ratio respectively).

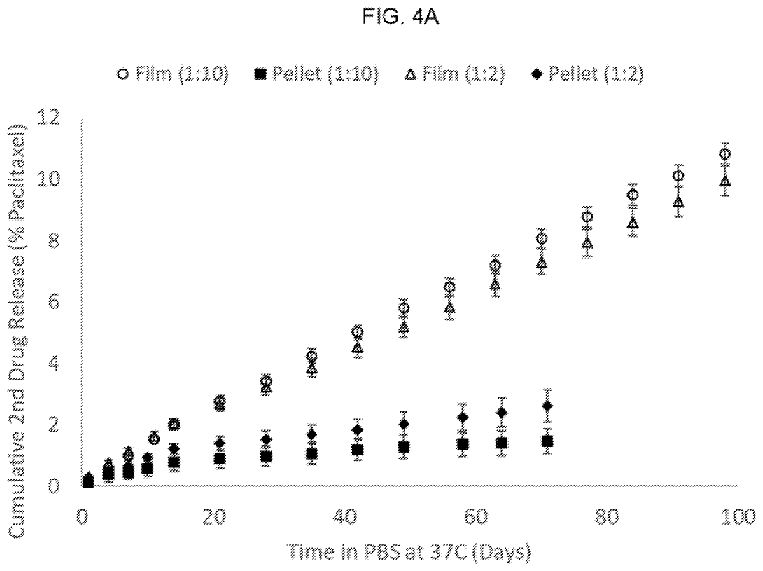

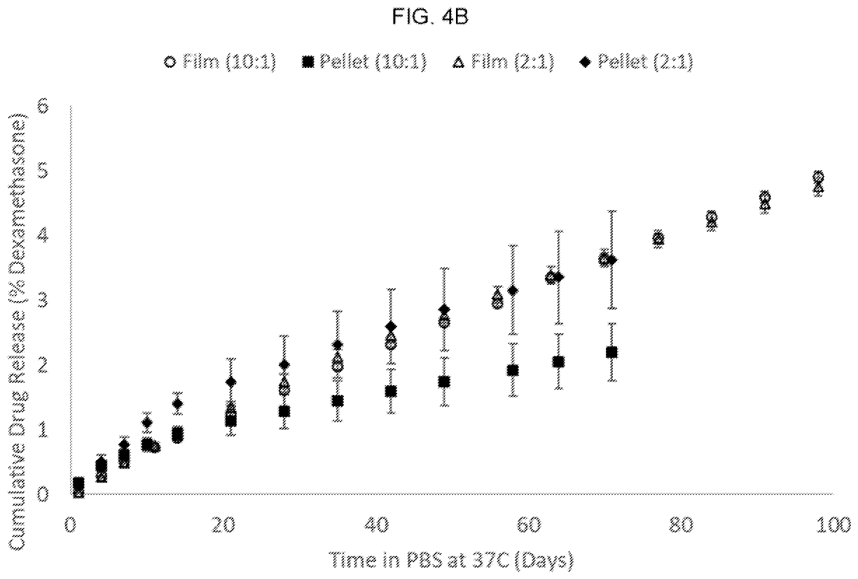

The coated Dacron films and heat-molded pellets with different ratios of compound 1 and paclitaxel were placed individually in 20 mL glass vials and 4 mL of phosphate buffered saline (PBS) was added. Samples were incubated at 37° C. on a shaker rotating at 115 rpm. After 1 day, 4 days, 7 days, 11 days, 14 days and subsequently in 7 day intervals, buffer was removed to quantify drug release from the films and pellets and was replaced with 4 ml of fresh buffer. Samples were analyzed by high performance liquid chromatography (HPLC) to quantify drug products. Cumulative drug release was calculated and plotted as a percentage of the total drug in each pellet released over time. FIG. 4A shows the released paclitaxel while FIG. 4B shows released dexamethasone from the films and pellets.

As observed in FIG. 4A and FIG. 4B, the rate of paclitaxel (filled square and filled diamond) release over 100 days varied between the films and pellets while the rate of dexamethasone (unfilled circle and unfilled triangle) released over 100 days was more similar between the two forms. While the rate of paclitaxel release as a percentage of the total was similar for the different ratios of drugs within the same form (e.g. 10:1 and 2:1 films), the total drug released at each time point (e.g., delivered dose) was correspondingly higher for the higher ratio of paclitaxel to compound 1. This allows the dose of paclitaxel to be tuned using ratio of paclitaxel to compound 1 while the duration can be tuned by the form and other physical properties such as surface area.

### Example 3: Combinations of Compound 1 (Dex-TEG-Dex) and Different Secondary Agents Can be Formed in Multiple Physical Forms Through Different Processing Techniques that Yield Sustained Release Formulations

Using conditions similar to those described in Example 2, a variety of active pharmaceutical ingredients were shown to be able to be formulated with compound 1 to give multiple physical forms that yield sustained release formulations when tested in vitro in the buffer PBS at 37 C. Table 1 and Table 2 summarizes the formed sustained release formulations with compound 1 and different secondary agents as well as respective drug release profiles.

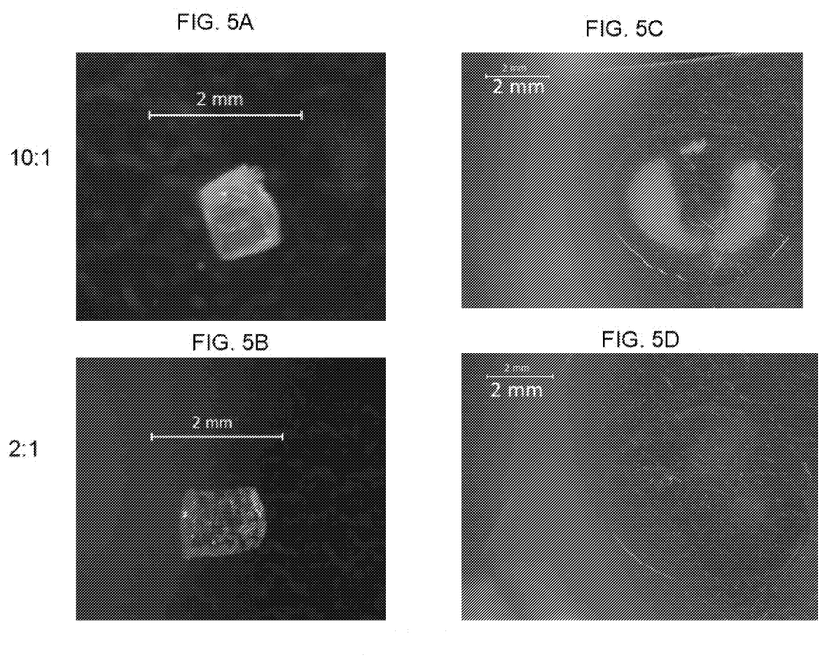

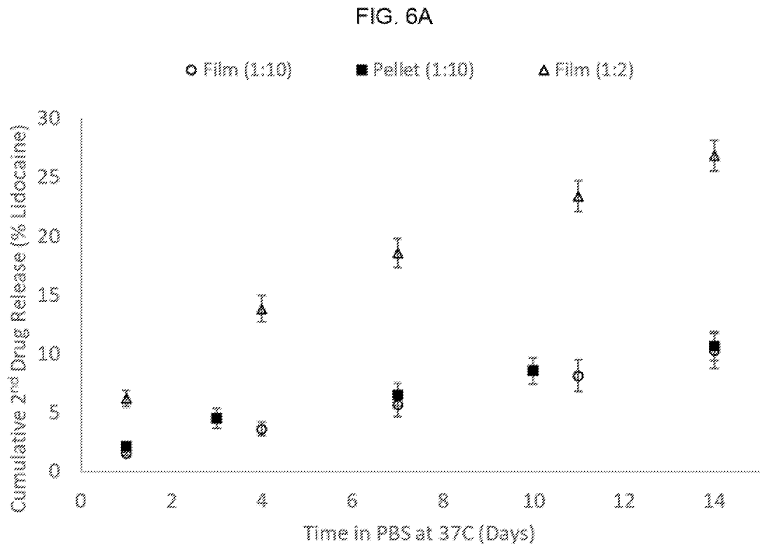

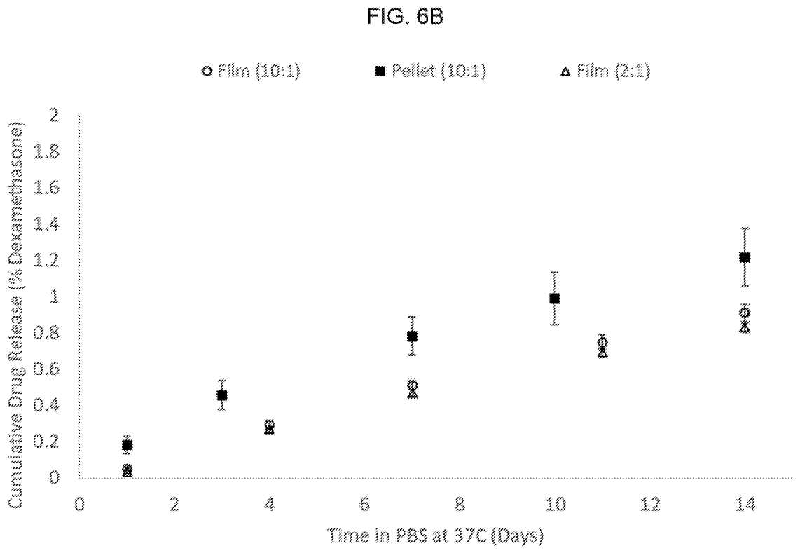

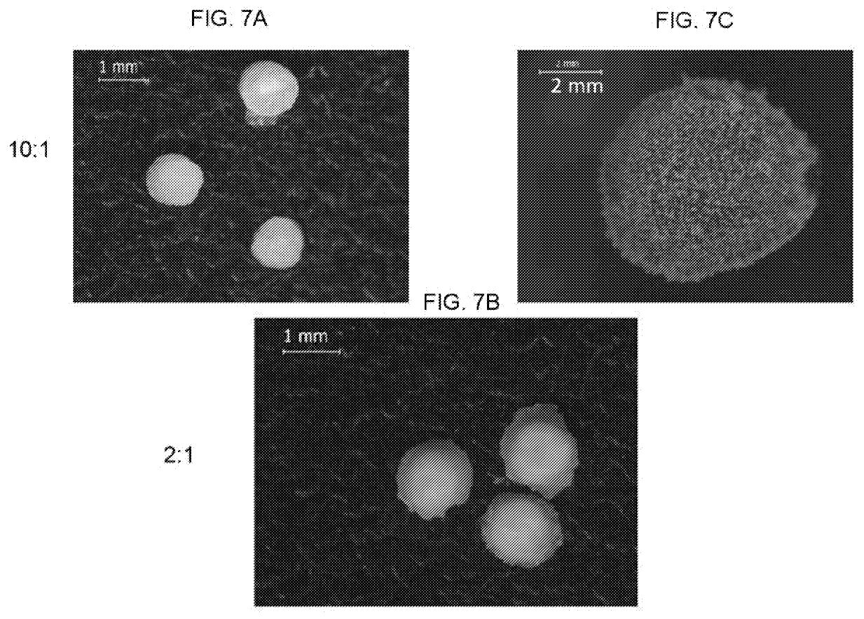

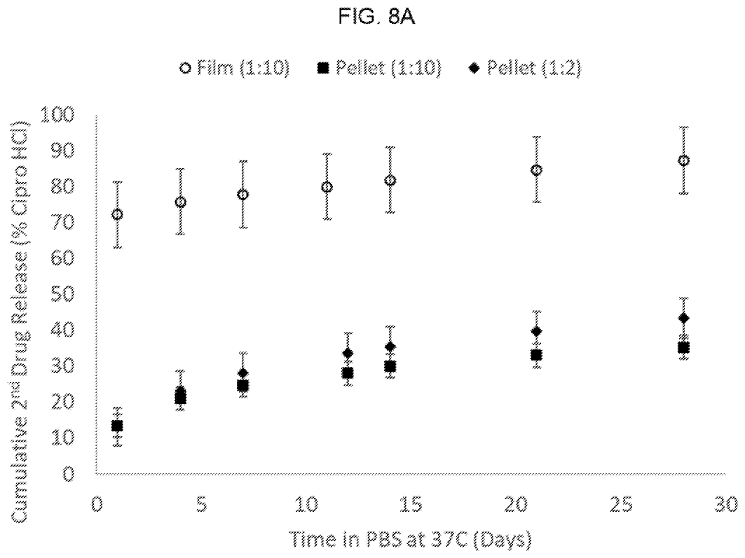

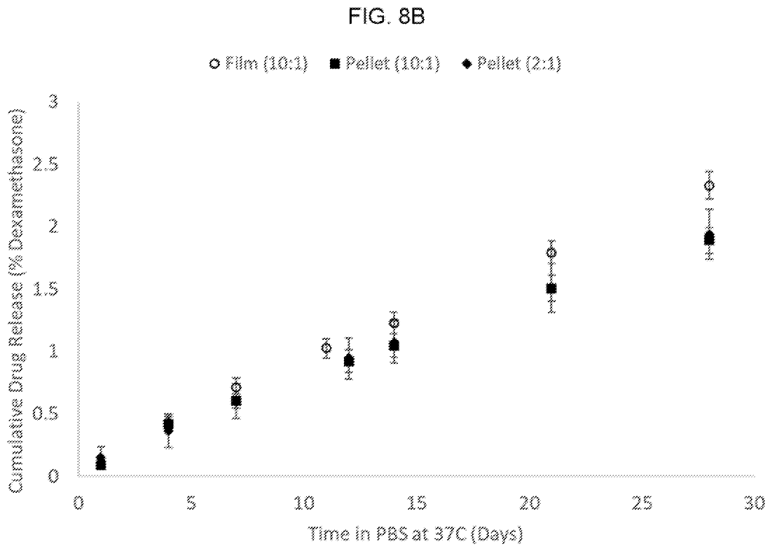

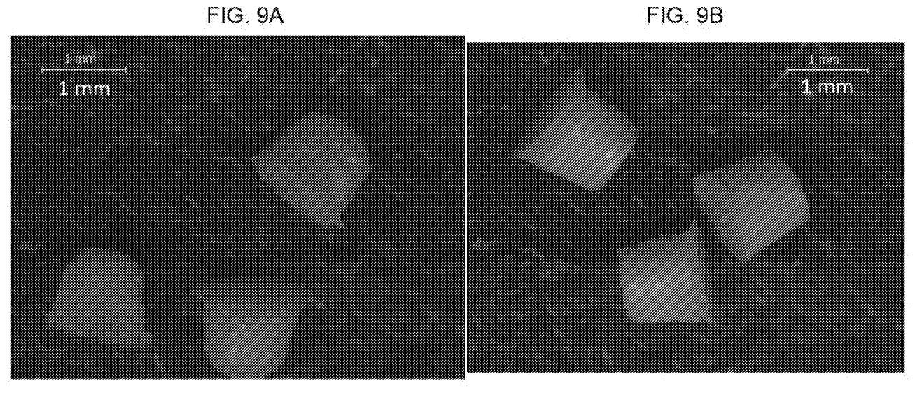

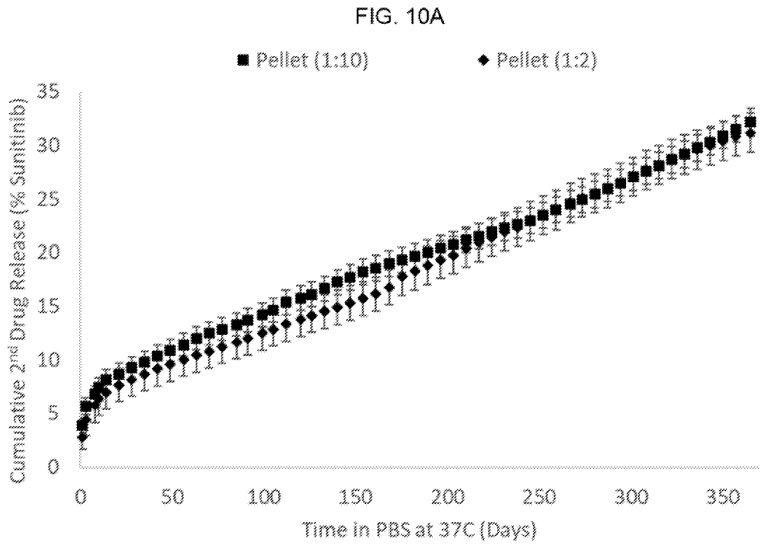

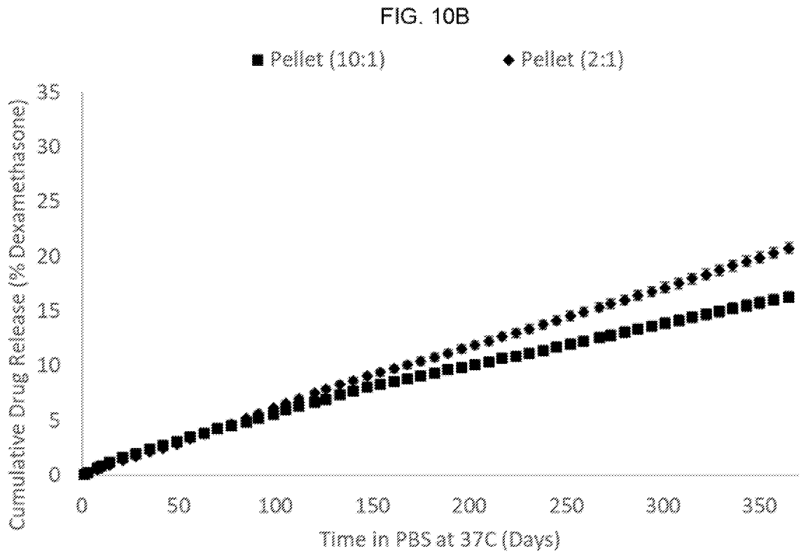

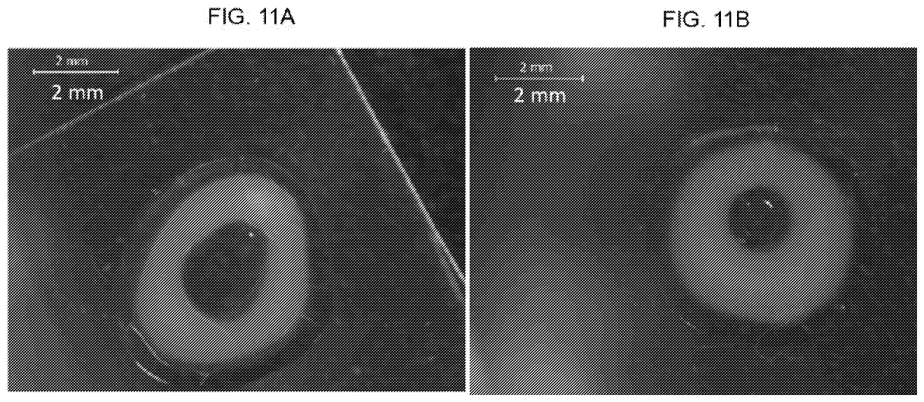

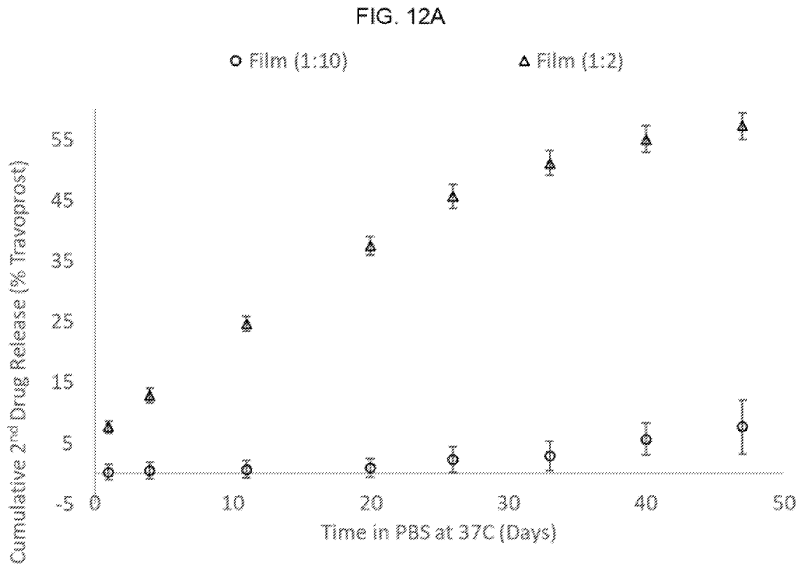

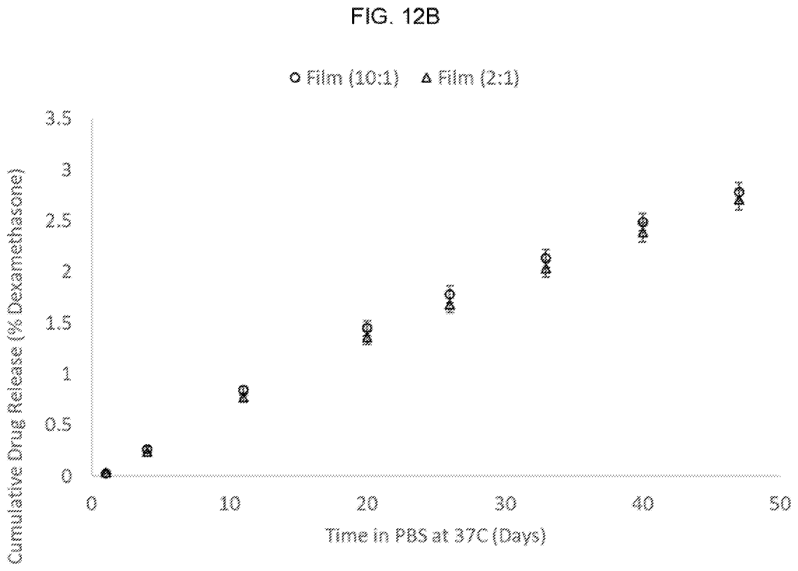

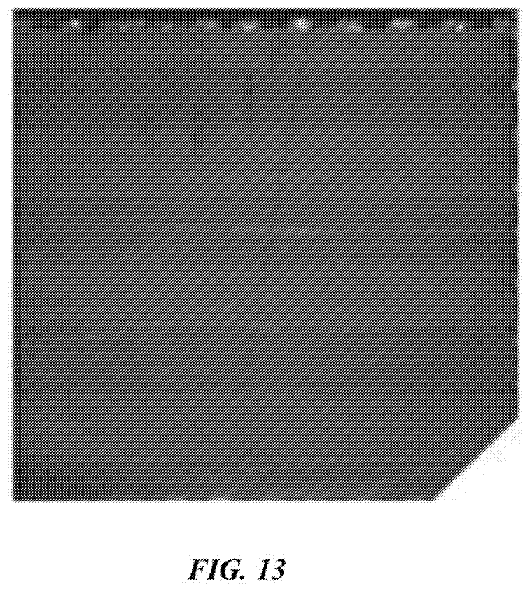

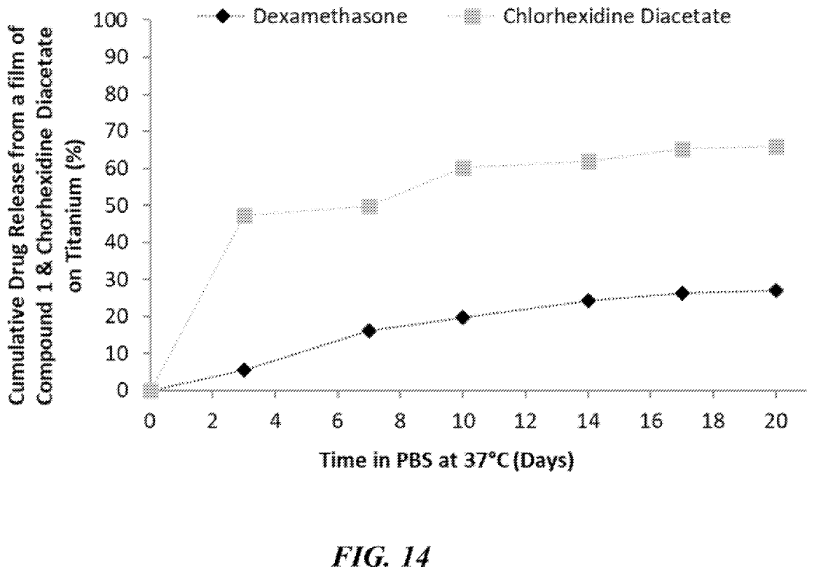

TABLE 1
Summary of Sustained Release Formulations with Compound 1

| 2^{nd} Agent | 10:1 | 2:1 | 10:1 | 2:1 | Drug Release | |
|---|---|---|---|---|---|---|
| (API) | Pellet | Pellet | Film | Film | 2^{nd} Agent | Dexamethasone |
| Lidocaine | FIG. 5A | FIG. 5B | FIG. 5C | FIG. 5D | FIG. 6A | FIG. 6B |
| Ciprofloxacin | FIG. 7A | FIG. 7B | FIG. 7C | N/A | FIG. 8A | FIG. 8B |
| HCl | | | | | | |
| Sunitinib | FIG. 9A | FIG. 9B | N/A | N/A | FIG. 10A | FIG. 10B |
| Malate | | | | | | |
| Travoprost | N/A | N/A | FIG. 11A | FIG. 11B | FIG. 12A | FIG. 12B |
| Chlorhexidine | N/A | N/A | N/A | FIG. 13 | FIG. 14 | FIG. 14 |
| Diacetate | | | | | | |

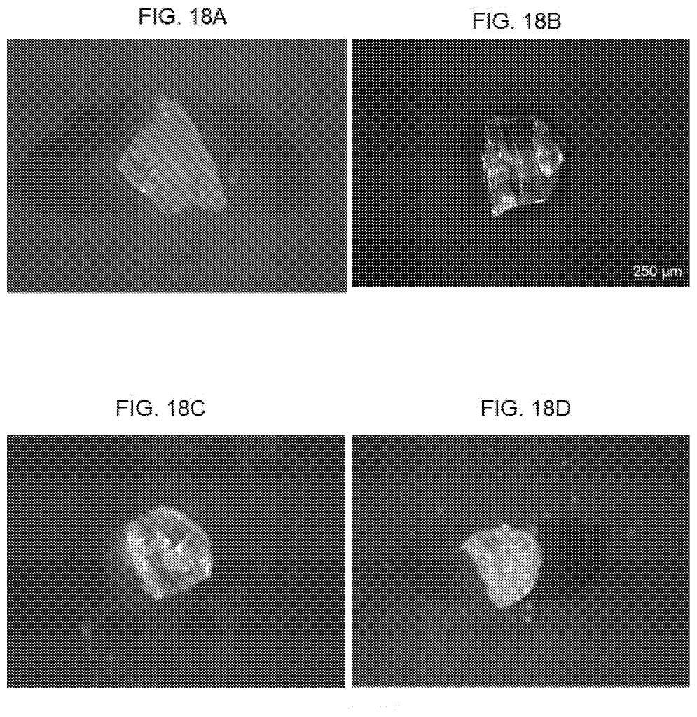

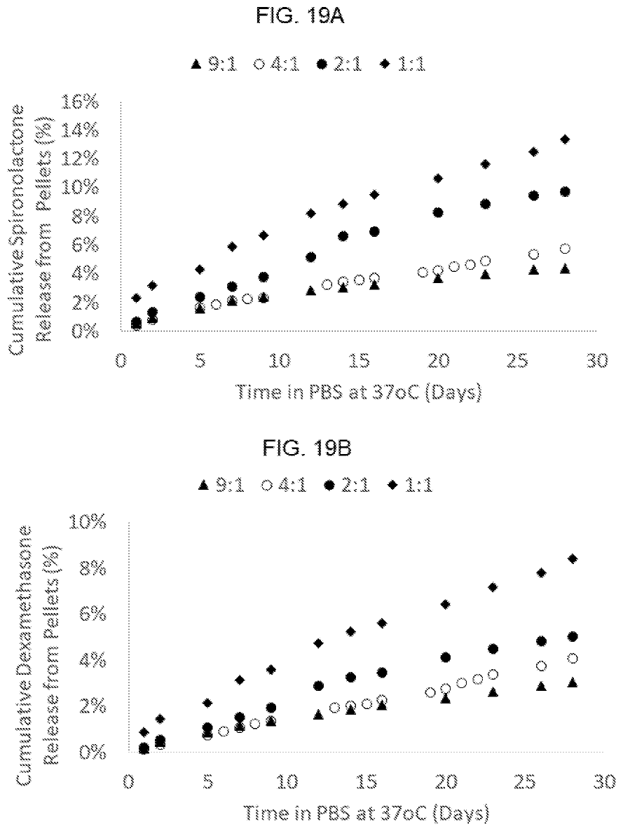

TABLE 2
Summary of Sustained Release Formulations with Compound 1 and Spironolactone

| 2^{nd} Agent | 9:1 | 4:1 | 2:1 | 1:1 | Drug Release | |
|---|---|---|---|---|---|---|
| (API) | Pellet | Pellet | Pellet | Pellet | 2^{nd} Agent | Dexamethasone |
| Spironolactone | FIG. 18A | FIG. 18B | FIG. 18C | FIG. 18D | FIG. 19A | FIG. 19B |

In forming solvent cast films, the solubility of both components must be maintained until the film is deposited on a surface or it could impact the mixing of secondary agent in the steroid dimer matrix and therefore impact the ability to form a sustained release formulation. This was observed for compound 1 and ciprofloxacin HCl formed into solvent cast films at the 10:1 ratio and led to an immediate release formulation instead of the intended sustained release formulation (FIG. 8A). This contrasted the heat molded pellets of the same formulation (drug combination and ratio), which had better second agent distribution within the dimer matrix and led to a more controlled sustained release formulation.

Solvent cast films of compound 1 and chlorhexidine diacetate showed a similar release trend as compound 1 and ciprofloxacin HCl (10:1) films, with a burst release of ^{˜}48% over the first few days and sustained release with a similar rate of release as dexamethasone out to 20 days thereafter (FIG. 14). The burst release was likely due to differences in solubility between compound 1 and chlorhexidine diacetate in the organic solvent used during film formation leading to roughly half the chlorhexidine diacetate on the surface and the rest distributed within the dimer matrix.

Pellets of compound 1 and sunitinib malate formed sustained release formulations and were shown to have similar rates of drug release over the course of a year independent of drug ratio (FIG. 10A). This is similar to what was observed with compound 1 and paclitaxel in Example 2 where the rate was the same but the daily dose released differed due to the different total amounts of free second agent. This provides a method to control daily dose by the selection of drug ratios while using other parameters, like form factor or surface area, to control duration.

Some drugs combined with compound 1 formed sustained release formulations that had drug release rates that corresponded with drug ratio. Compound 1 and lidocaine (FIG. 6A), travoprost (FIG. 12A), and spironolactone (FIG. 19A) all showed this behaviour, thereby providing a mechanism to control release rate through selection of drug ratio. Interestingly, while the dexamethasone released in these formulations was largely independent of drug ratio (and form if tested) for lidocaine (FIG. 6B) and travoprost (FIG. 12B), spironolactone (FIG. 19B) showed a relation to dexamethasone release and drug ratio. For example, spironolactone is a member of the steroid family (mineralocorticoid receptor antagonist) and the structural similarities to the steroid dimer may have influenced the dimer solubility, leading to increases in rate of dexamethasone release with increased spironolactone.

### Example 4: Combinations of Compound 1 (Dex-TEG-Dex) and Different Secondary Agents Can be Heat Processed into Thin Fibers Directly from the Crystalline State or Using an Intermediate Step from the Solution State

Thin fibers were prepared from compound 1 and different secondary agents using heat processing techniques. Mixtures of compound 1 and the secondary agents were prepared by weighing and mixing the starting powders as described in Example 2. The powders were heated to 185° C. on a flat plate to melt compound 1 and the resulting tacky mixture was pulled at different rates with fine-point tweezers to yield fibers of different diameters. Table 3 summarizes the formulations and ratios used to pull fibers. For select combinations where the second drug is not a solid, for example travoprost, an intermediate step was used to mix the two components, similar to the solvent cast films procedure in Example 2. The drop casted thin film of compound 1 and the second agent (e.g. travoprost) was then heated to a temperature above the glass transition temperature of compound 1 (e.g., 150 C) and fibers were pulled from the viscous mixture.

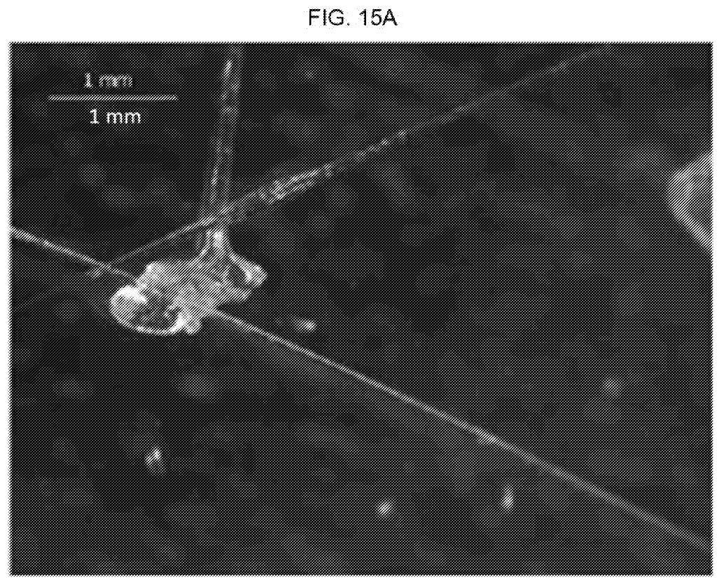

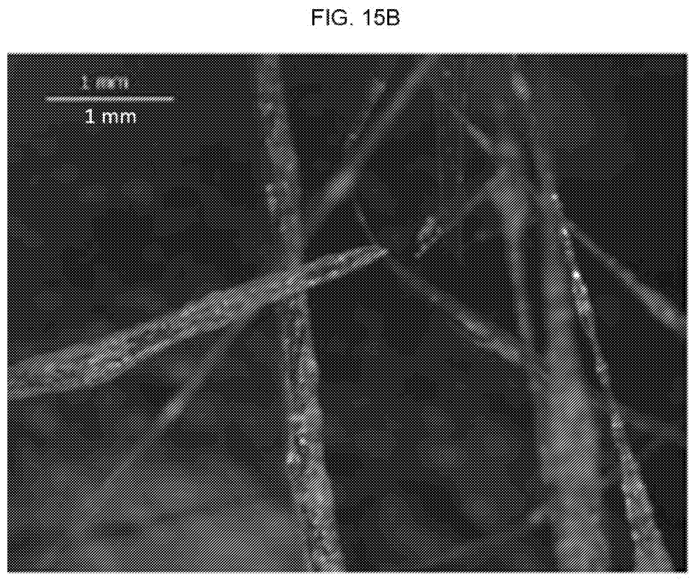

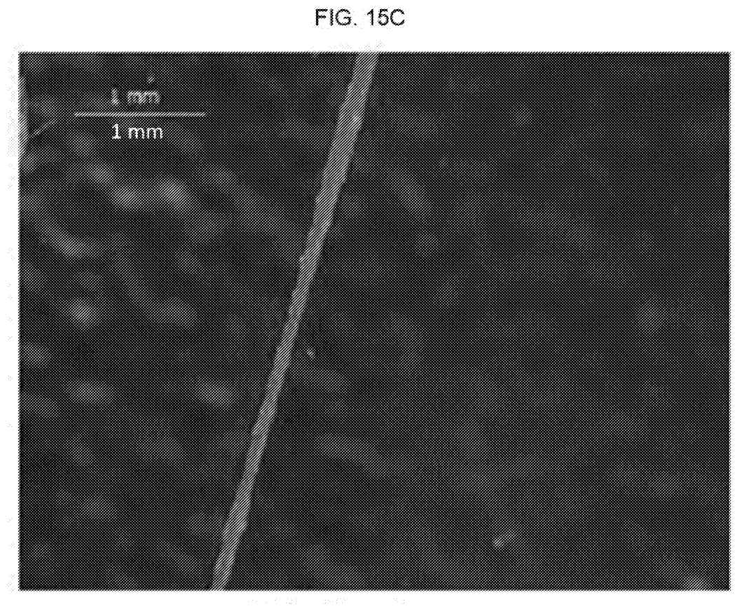

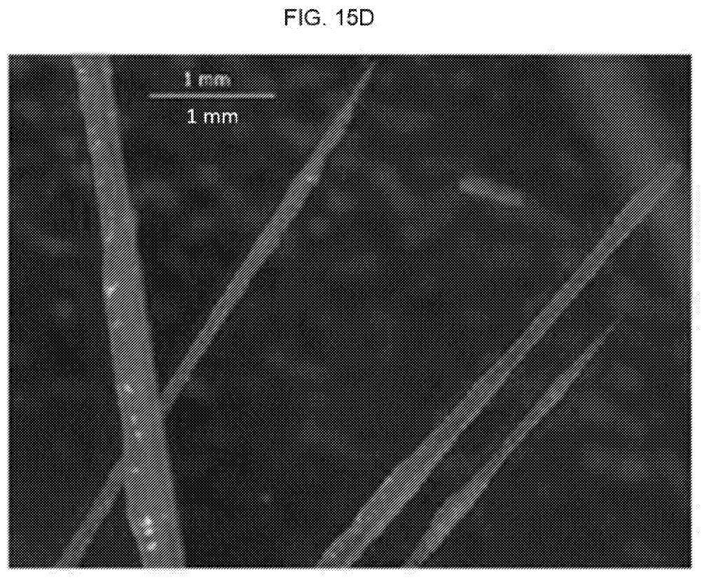

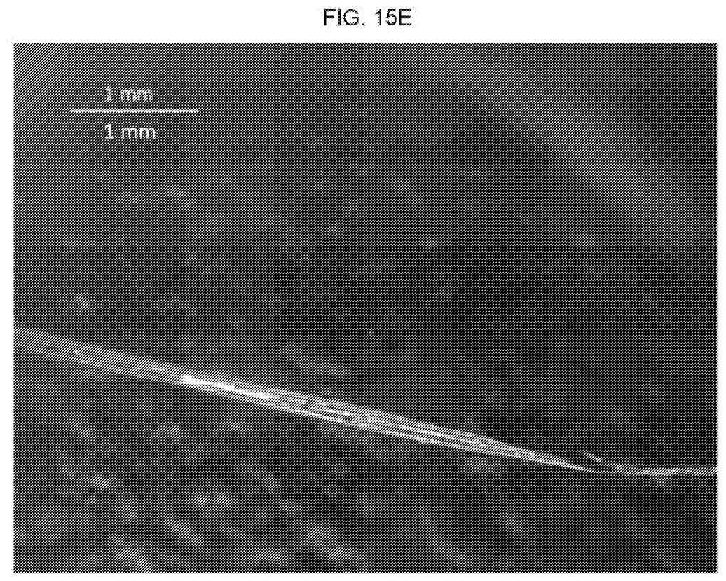

TABLE 3
Summary of Heat-Processed Formulations Formed into Fibers

| | Ratio of 2^{nd} Agent to | Representative |
|---|---|---|
| 2^{nd} Agent (API) | Compound 1 | Images |
| Lidocaine | 1:10 | FIG. 15A |
| Ciprofloxacin HCl | 1:10 | FIG. 15B |
| Sunitinib Malate | 1:10 | FIG. 15C |
| | 1:2 | FIG. 15D |
| Travoprost | 1:10 | FIG. 15E |

### Example 5: A Sustained Release Formulation Consisting of Compound 2 (Hydrocortisone-Triethylene Glycol-hydrocortisone; HC-TEG-HC) and the Non-Steroidal Anti-Inflammatory Agents (NSAIDs) Ibuprofen and Naproxen Can be Formed from Solvent Cast Films

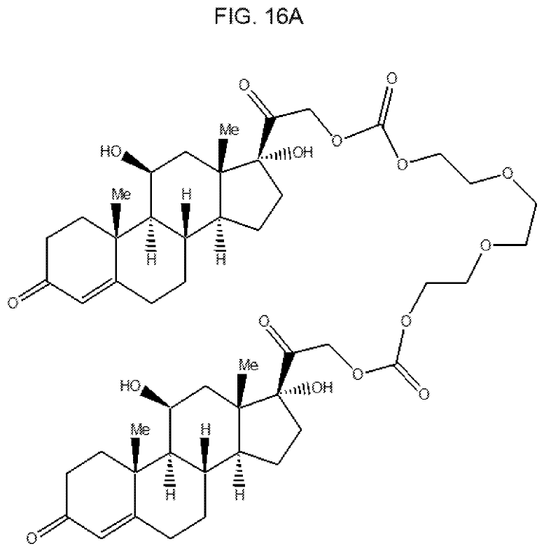

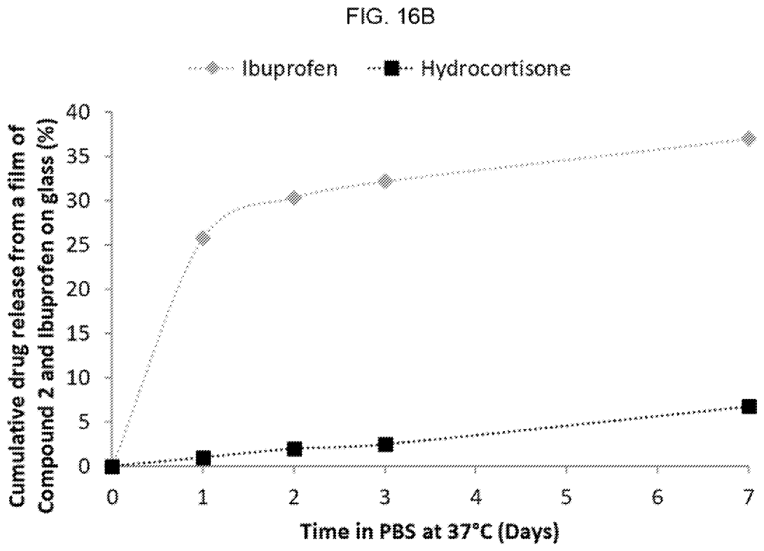

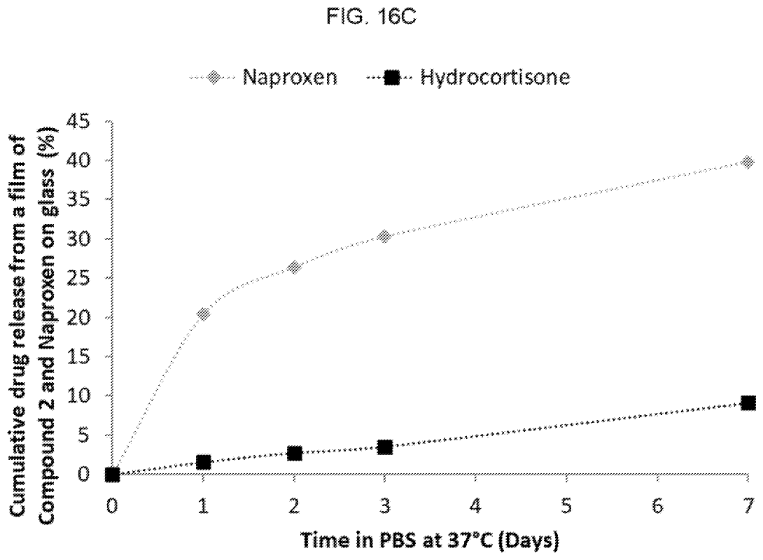

Compound 2 (HC-TEG-HC; FIG. 16A) was synthesized using a similar procedure to that described for compound 1 in Example 1. Compound 2 and the NSAIDs (either ibuprofen or naproxen) were mixed together at a 10:1 w/w ratio of compound 2:NSAID, were dissolved in dichloromethane (DCM), cast onto the bottom of a glass vial, and the solvent was evaporated to give a total of 2 mg compound 2 and 0.2 mg of NSAID. The release buffer PBS (8 mL) was added to the glass vial and drug release was monitored by HPLC at different time points over 1 week after incubation at 37 C with buffer changes at each time point. The films gave an initial burst release of NSAID and then a sustained release of NSAID thereafter (e.g., for ibuprofen (FIG. 16B) and naproxen (FIG. 16C)). The difference may be a result of the difference in solubility of ibuprofen and naproxen Sustained release of hydrocortisone from compound 2 was observed without the burst release in both formulations.

### Example 6: Formulations with Compound 3 (cholesterol-triethylene glycol-cholesterol; CHS-TEG-CHS) and Secondary Agents can be Formed but do not Release Drug

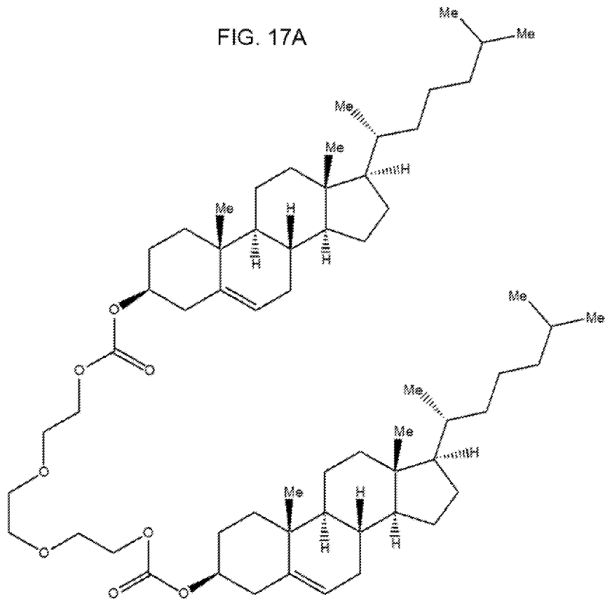

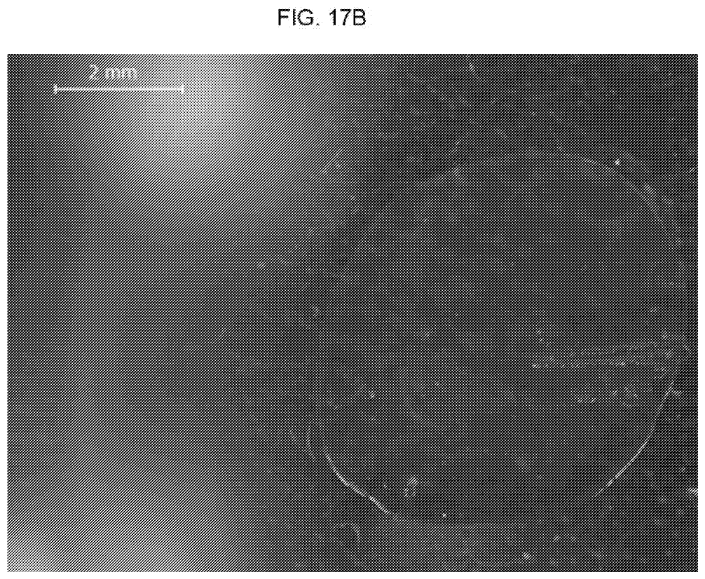

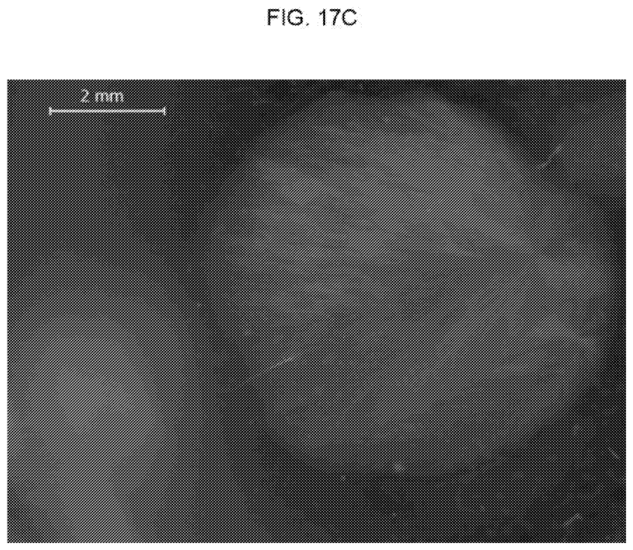

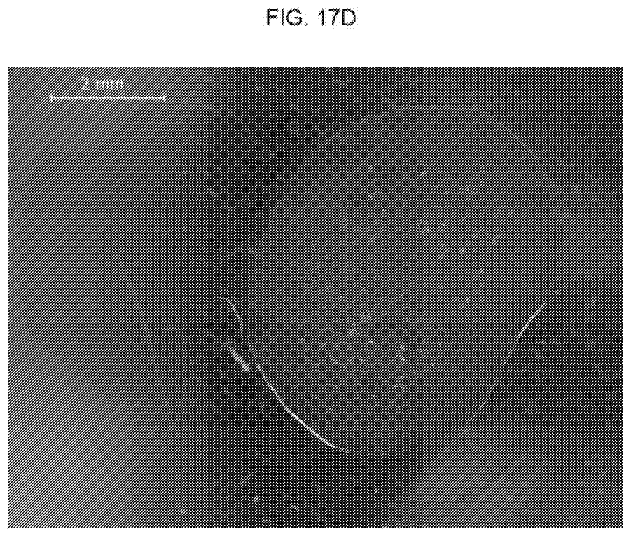

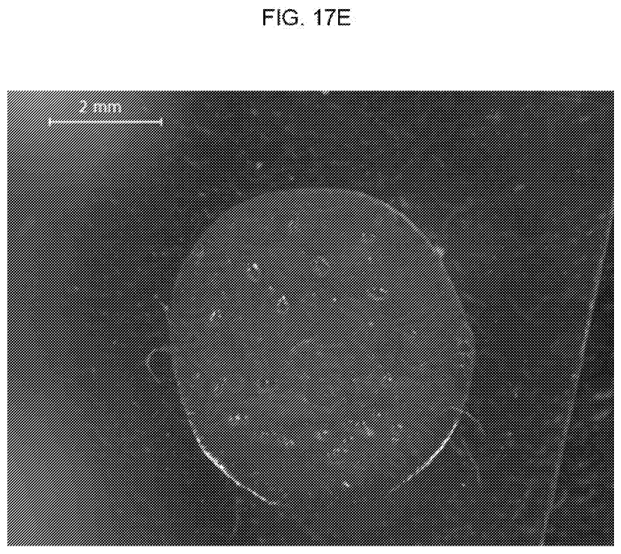

Compound 3 (CHS-TEG-CHS; FIG. 17A) was synthesized using a similar procedure to that described for compound 1 in Example 1. Films were cast using the procedure outlined in Example 2 using tetrahydrofuran (THF) as the solvent with compound 3 and lidocaine at a 10:1 (FIG. 17B) or 2:1 (FIG. 17C) ratio or paclitaxel at a 10:1 (FIG. 17D) or 2:1 (FIG. 17E) ratio. Release studies were performed similar to Example 2 but no release was observed from any of the formulations with the exception of a small initial burst release of lidocaine. The lack of sustained release was likely due to the insolubility of compound 3 used as the matrix as similar films formed with compound 1 and the same secondary agents (see Examples 2 & 3) resulted in sustained release.

### Example 7: Layered Cylinder Containing a Core Formed from Compound 1 (Dex-TEG-Dex) and an Outer Layer Formed from a Mixture of Compound 1 and Dexamethasone

A cylinder core is formed from Compound 1 alone by heat extrusion from the melt or from an intermediate glassy state. To the surface of the cylinder core is applied a coating layer formed from a mixture of 65% (w/w) dexamethasone and 35% (w/w) Compound 1 in solution followed by quickly evaporating the solvent to form the outer layer. Alternatively, the cylinder core of compound 1 and the outer layer of dexamethasone and compound 1 (at 65:35% w/w) are formed by co-extrusion at a temperature above the Tm of compound 1 for crystalline starting compound 1 or at a temperature above the Tg of compound 1 for an intermediate glassy state form of compound 1.

The resulting layered cylinder can produce a multi-phased dexamethasone release profile, where the coating layer provides an immediate release of dexamethasone upon administration and the cylinder core provides an extended release of dexamethasone.

Such formulations can be useful, for example, for treating active acute or chronic inflammation at a site during the first phase (immediate release of dexamethasone), which is followed by maintenance therapy that reduces the risk of recurring inflammation during the second phase (sustained release of dexamethasone) phase of dexamethasone release.

### Example 8: Sustained Release Formulations Consisting of Compound 4 (Prednisolone-Triethylene Glycol-Prednisolone; Pred-TEG-Pred) and the Non-Steroidal Anti-Inflammatory Agents (NSAIDs) Diclofenac Sodium Salt, Diclofenac, and Flurbiprofen can be Formed by Heat Processing

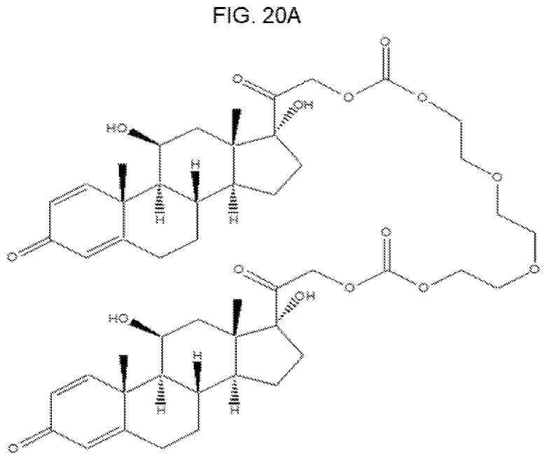

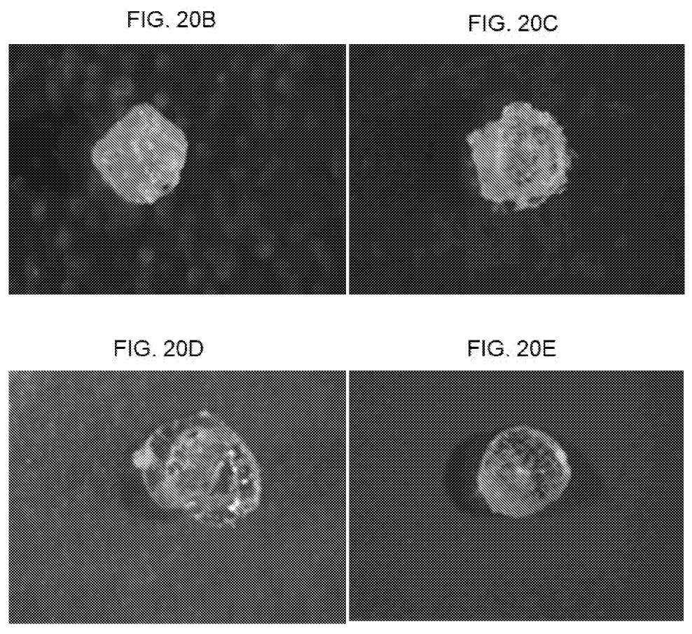

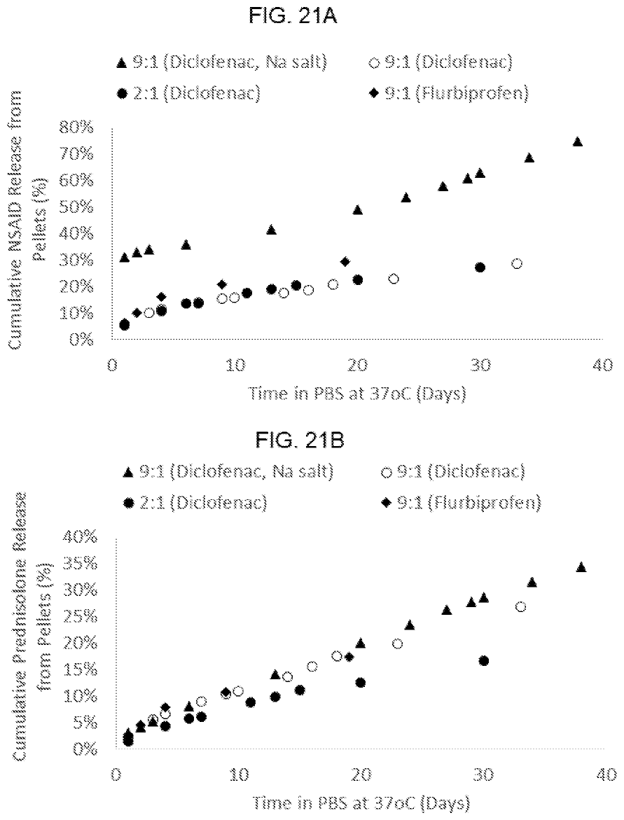

Compound 4 (Pred-TEG-Pred; FIG. 20A) was synthesized using a similar procedure to that described for compound 1 in Example 1. Using conditions similar to those described in Example 2 at a temperature of 150° C., different NSAIDs were formulated with compound 4 to provide pellets (FIG. 20B-20E) that yield sustained release formulations when tested in vitro in the buffer PBS at 37° C. (FIG. 21A and FIG. 21B). Table 4 summarizes the formed sustained release formulations with compound 4 and different NSAIDs as well as respective drug release profiles.

TABLE 4
Summary of Sustained Release Formulations

| | | | Drug Release | |
|---|---|---|---|---|
| 2^{nd} Agent (API) | 9:1 Pellet | 2:1 Pellet | 2^{nd} Agent | Prednisolone |
| Diclofenac Sodium Salt | FIG. 20B | N/A | FIG. 21A | FIG. 21B |
| Diclofenac | FIG. 20C | FIG. 20D | FIG. 21A | FIG. 21B |
| Flurbiprofen | FIG. 20E | N/A | FIG. 21A | FIG. 21B |

The sustained release formulations with NSAIDs and compound 4 exhibited similar drug release properties (FIG. 21A) as observed with different formulations from compound 1. NSAID release rates were independent of drug ratio as seen with similar release rates for 9:1 (unfilled circle) and 2:1 (filled circle) ratios of compound 4 and diclofenac (FIG. 21), thus providing a mechanism to control dose (through drug ratio) and duration through physical properties. When the NSAID was unevenly distributed within the pellet (e.g., forming a layer of higher NSAID content on the surface, as seen with compound 4 and diclofenac sodium salt (filled triangle)), an initial burst release was observed with sustained release thereafter. The rate of drug release for the sustained release behavior of the diclofenac sodium salt formulation once the burst release was completed mimicked the rate of drug release for the other NSAIDs. The formulations exemplify how to control drug release properties to achieve a desired release profile.

1-27. (canceled)

28. An article comprising a steroid material and at least one therapeutic agent, the steroid material comprising a compound of formula (A-I):
D1-L-D2 (A-I)
or a pharmaceutically acceptable salt thereof,
wherein:
(i) each of D1 and D2 is, independently, a steroid radical; and L is a linker covalently linking D1 to D2; and
(ii) the at least one therapeutic agent is distributed throughout the steroid material.

29. The article of claim 28, wherein the steroid material is free of a controlled release excipient.

30. The article of claim 28, wherein the article comprises 45% to 99% (w/w) of the compound of formula (A-I).

31. The article of claim 30, wherein the article comprises more than or equal to 90% (w/w) of the compound of formula (A-I).

32. The article of claim 28, wherein the article comprises 1% to 55% (w/w) of the at least one therapeutic agent.

33. The article of claim 32, wherein the article comprises less than or equal to 10% (w/w) of the at least one therapeutic agent.

34. The article of claim 28, wherein the article has a (w/w) ratio of the compound of formula (A-I) to the at least one therapeutic agent of 20:1 to 1:1

35. The article of claim 28, wherein the article has a (w/w) ratio of the compound of formula (A-I) to the at least one therapeutic agent of 10:1 to 2:1.

36. The article of any one of claim 28, wherein the at least one therapeutic agent is released with at least a portion of the steroid material.

37. The article of claim 28, wherein D1 and D2 are released from the article in their free form at a rate such that t₁₀ is greater than or equal to 1/10 of t₅₀.

38. The article of claim 28, wherein a free form of D1, a free form of D2, the at least one therapeutic agent, or any combination thereof is released from the article through surface erosion.

39. The article of claim 28, wherein less than or equal to 20% (w/w) of D1, D2, or both D1 and D2 are released from the article in their free form at 37° C. in PBS over a period 5 days or more.

40. The article of claim 39, wherein more than or equal to 50% (w/w) of the at least one therapeutic agent is released from the article at 37° C. in PBS over a period 1 day or more.

41. The article of claim 28, wherein less than or equal to 20% (w/w) of the at least one therapeutic agent is released from the article at 37° C. in PBS over a period 2 days or more.

42. The article of claim 28, wherein the article comprises a first portion of the steroid material, which comprises the at least one therapeutic agent distributed throughout the steroid material, and a second portion of the steroid material, which is free of the at least one therapeutic agent.

43. The article of claim 28, wherein D1 and D2 are each independently an anti-inflammatory steroid, or a pharmaceutically acceptable salt thereof, in their free form.

44. The article of claim 28, wherein the at least one therapeutic agent comprises a first therapeutic agent and a second therapeutic agent.

45. A method for treating a disease or disorder in an individual, the method comprising implanting the article of claim 28 into the individual.

46. The method of claim 45, wherein at least one component of the article is released into the individual for a period of more than or equal to 2 days.

47. The method of claim 45, wherein the disease or disorder is a chronic condition.
